(19) United States
(12) Patent Application Publication
(10) Pub. No.: US 2021/0244796 A1
(43) Pub. Date: Aug. 12, 2021

(54) COMPOSITIONS IN THE FORM OF AN INJECTABLE AQUEOUS SOLUTION COMPRISING AMYLIN AN AMYLIN RECEPTOR AGONIST OR AN AMYLIN ANALOG, AT LEAST ONE IONIC SPECIES AND AN AMPHOIPHILIC COMPOUNDS CONTAINING HYDRIPHOBIC RADICALS

(71) Applicant: ADOCIA, Lyon (FR)

(72) Inventor: Emmanuel DAUTY, Lyon (FR)

(73) Assignee: ADOCIA, Lyon (FR)

(21) Appl. No.: 17/263,676

(22) PCT Filed: Aug. 3, 2019
(86) PCT No.: PCT/EP2019/070954

§ 371 Date: Jan. 27, 2021

(30) Foreign Application Priority Data
Aug. 3, 2018 (FR) 18/57266

(51) Int. Cl. A61K 38/22; A61K 9/00; A61K 47/64; A61K 38/26

(52) U.S. Cl. CPC A61K 38/22; A61K 38/26; A61K 47/6455; A61K 9/0019

(57) ABSTRACT

A composition in the form of an injectable solution includes: amylin, an amylin receptor agonist or an amylin analog; at least one ionic species; and an amphiphilic compound having a hydrophilic skeleton HB, substituted by at least one hydrophobic radical -Hy according to formula (I). A composition further is characterised in that it also has prandial insulin. In one embodiment, the composition also has GLP-1, GLP-1 analogs, and GLP-1 receptor agonists, commonly called GLP-1 RA.

This invention relates to amylin, amylin receptor agonist or amylin analog injection therapies for treating diabetes.

The invention relates to physically stable compositions in the form of an injectable aqueous solution, the pH of which is from 6.0 to 8.0, comprising at least amylin: an amylin receptor agonist or an amylin analog and an amphiphilic compound comprising a hydrophilic backbone HB bearing hydrophobic radicals according to the invention, and compositions further comprising an insulin (excluding basal insulins whose isoelectric point pI is from 5.8 to 8.5). The invention also relates to pharmaceutical formulations comprising the compositions according to the invention. Finally, the invention also relates to a use of amphiphilic compounds comprising a hydrophilic backbone HB bearing hydrophobic radicals. according to the invention, for stabilizing amylin, amylin receptor agonist or amylin analog compositions as well as amylin, amylin receptor agonist or amylin analog compositions further comprising an insulin.

In one embodiment, the composition according to the invention does not include basal insulin whose isoelectric point p1 is from 5.8 to 8.5, and in particular no insulin glargine.

Type 1 diabetes is an autoimmune disease leading to the destruction of beta cells in the pancreas. These cells are known to produce insulin, the main role of which is to regulate the use of glucose in peripheral tissues (Gerich 1993 Control of glycaemia). Therefore, patients with type 1 diabetes suffer from chronic hyperglycemia and must administer exogenous insulin in order to limit this hyperglycemia. Insulin therapy has drastically changed the life expectancy of these patients. However, glycemic control provided by exogenous insulin is not optimal, especially after taking a meal. This is bound to the fact that these patients produce glucagon after taking a meal, which leads to the release of part of the glucose stored in the liver, which is not the case with the healthy person. This glucagon-mediated glucose production exacerbates the problem of blood sugar regulation in these patients.

It has been shown that amylin, another hormone produced by beta cells in the pancreas and therefore also deficient in type 1 diabetic patients, plays a key role in the regulation of post-prandial blood sugar. Amylin, also known as “islet amyloid polypeptide” or IAPP, is a 37 amino acid peptide that is co-stored and co-secreted with insulin (Schmitz 2004 Amylin Agonists). This peptide is described to block the production of glucagon by alpha cells in the pancreas. Thus, insulin and amylin have complementary and synergistic roles since insulin makes it possible to reduce the concentration of glucose in the blood while amylin makes it possible to reduce the entry of endogenous glucose into the blood by inhibiting the production (secretion) of endogenous glucagon.

This problem to regulate postprandial blood sugar is quite similar for patients with type 2 diabetes treated with insulin as their disease has led to a very significant loss of their beta cell mass and therefore, their capacity to produce insulin and amylin.

Human amylin has properties which are not compatible with pharmaceutical requirements in terms of solubility and stability (Goldsbury C S, Cooper G J, Goldie K N, Muller S A, Saafi E L, Gruijters W T, Misur M P, Engel A, Aebi U, Kistler J: Polymorphie fibrillar assembly of human amylin. J Struct Biol 119:17-27, 1997). Amylin is known to form amyloid fibers which lead to the formation of plaques which are insoluble in water. Although being the natural hormone, it was necessary to develop an analogue in order to solve these solubility problems.

Thus, the physicochemical properties of amylin make its use impossible: Amylin is only stable for about fifteen minutes at acidic pH, and less than one minute at neutral pH.

The company Amylin, has developed an analogue of amylin, pramlintide, to overcome the lack of stability of human amylin. This product, marketed under the name Symlin, was approved in 2005 by the FDA for the treatment of type 1 and type 2 diabetics. It should be administered subcutaneously three times a day, within one hour of a meal to improve post-prandial blood sugar control. This peptide is formulated at acidic pH and is described as shining when the pH of the solution is greater than 5.5. Variant analogs are described in U.S. Pat. No. 5,686,411.

This analogue is thus not satisfactory from the point of view of stability when a formulation at neutral pH is contemplated.

To date, there is no way to stabilize human amylin in order to make it into a pharmaceutical product. However, it would be advantageous for patients to have access to the human form of this physiological hormone. It would also be advantageous to be able to formulate an amylin receptor analog or agonist at neutral pH.

In addition, there would be an advantage in being able to mix amylin in aqueous solution, an amylin analogue, or an amylin receptor agonist, with a prandial insulin since these two products are to be administered before the meal. This would also make it possible to mimic physiology since these two hormones are co-secreted by beta cells in response to a meal to improve post-prandial blood sugar control.

However, taking into account the fact that the solutions of prandial insulins have a pH close to neutral for reasons of chemical stability, it is not possible to obtain an aqueous solution that would meet pharmaceutical requirements in terms of solubility and stability.

For this reason, patent application US2016/001002 from the ROCHE company describes a pump containing two separate reservoirs in order to make it possible to co-administer these two hormones with a single medical device. However, this patent does not solve the problem of mixing these two hormones in solution. which would allow them to be administered with conventional pumps already on the market which only contain one reservoir.

The patent application WO2013067022 from the company XERIS provides a solution to the problem of stability of amylin and its compatibility with insulin by using an organic solvent in place of water. The absence of water seems to solve the stability problems, but the use of an organic solvent poses chronic safety problems for diabetic patients and also compatibility problems with the usual medical devices, at the tubing, gaskets and plasticizers used.

Patent application WO2007104786 of the company NOVO NORDISK, describes a method for stabilizing a pramlintide solution, which is an analogue of amylin, and insulin by adding a phospholipid, glycerophosphoglycerol derivative, in particular dimyristoyl glycerophosphoglycerol (DMPG). However, this solution requires the use of large amounts of DMPG which may pose a problem of local tolerance.

To the knowledge of the plaintiff, there is no satisfactory way to make it possible to combine a prandial insulin and human amylin, in an aqueous solution, an amylin receptor agonist or an amylin analogue so that it can be administered with conventional devices.

The acid pH formulation and rapid fibrillation put the brakes on obtaining a pharmaceutical formulation at a neutral pH based on amylin and pramlintide, but they also put the brakes on combining amylin or pramlintide with other active pharmaceutical ingredients, in particular with peptides or proteins.

A traditional method for measuring the stabilities of proteins or peptides consists of measuring the formation of fibrils using Thioflavin T, also called ThT. This method makes taking measurements under conditions of temperature and agitation possible, which allows for an acceleration of the phenomenon, the latency time before the formation of fibrils, by measuring the increase in fluorescence. The compositions according to the invention have a latency time before the formation of fibrils that is markedly greater than that of amylin, an amylin receptor agonist or an amylin analog at the pH of interest.

This invention seeks to provide novel amphiphilic compounds comprising a hydrophilic backbone HB comprising one or more hydrophobic grafts, said grafts comprising one or more imidazole radicals. These compounds make it possible to have a modular association with amylin, an amylin receptor agonist or an amylin analogue and to also obtain compositions comprising amylin, an amylin receptor agonist or an amylin analogue which are stable.

By modulable association is meant that the association of said hydrophilic backbone HB with amylin, an amylin receptor agonist or an amylin analogue, may be more or less strong depending on the environment of said amphiphilic compound.

The invention thus relates to a composition in the form of an injectable solution, comprising:
amylin, an amylin receptor agonist or an amylin analogue,
at least one ionic species, and
an amphiphilic compound comprising a hydrophilic backbone HB, substituted by at least one hydrophobic radical -Hy according to formula I.

The invention also relates to a composition, in the form of an injectable solution, comprising:
amylin, an amylin receptor agonist or an amylin analogue,
at least one ionic species, in particular an at least divalent cation salt, and
an amphiphilic compound comprising a hydrophilic backbone HB, substituted by at least one hydrophobic radical -Hy according to formula I.

The invention also relates to a composition, in the form of an injectable solution, comprising:
amylin, an amylin receptor agonist or an amylin analog,
at least one ionic species, and
an amphiphilic compound comprising a hydrophilic backbone HB, substituted by at least one hydrophobic radical -Hy according to the following formula I:

*-(GpR)ᵣ-(GpI)ᵢ-[(GpR)ᵣ [symbol] (GpI)ᵢ [symbol] ]ₜ-GpC Formula I

wherein,
GpI is a divalent radical, said radical comprising at least one imidazole Im unit of Formula III:

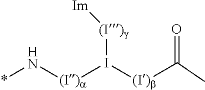

GpR is a radical according to formulas II, II or II″:

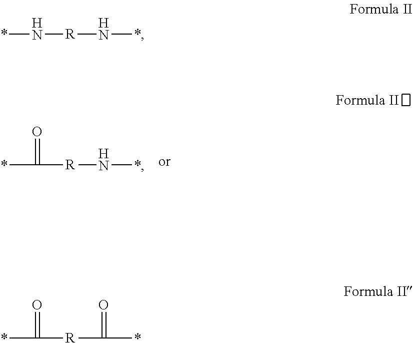

GpC is a radical according to formulaIV:

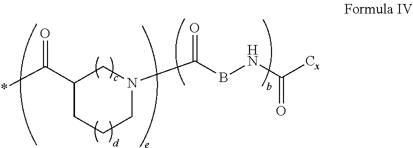

the * indicate the binding sites of the hydrophobic radical -Hy to the hydrophilic backbone HB or the above radicals (I, II, II [symbol] , II″, III and IV) with each other via amide functions ;
α, β and γ are identical or different integers equal to 0 or 1;
b is an integer equal to 0 or 1;
c is an integer equal to 0 or 1;
d is an integer equal to 0, 1 or 2; and if c is equal to 0 then d is equal to 1 or 2;
e is an integer equal to 0 or to 1;
i and i, whether they are identical or different, are integers less than or equal to 6 and i+i [symbol] is greater than or equal to 1 and less than or equal to 6, 1≤i+i [symbol] 6,
r and r [symbol] are integers equal to 0, 1, 2 or 3;
if r is equal to 0 then the hydrophobic radical according to formula I is bound to the hydrophilic backbone HB via a covalent bond between a carbonyl of the hydrophobic radical and a nitrogen atom of the hydrophilic backbone HB, thus forming an amide function resulting from the reaction of an amine function of the precursor of the hydrophilic backbone HB and an acid function borne by the precursor of the hydrophobic radical, and
if r is equal to 1, 2 or 3 then the hydrophobic radical -Hy according to formula I is bound to the hydrophilic backbone HB:
   via a covalent bond between a nitrogen atom of the hydrophobic radical and a carbonyl of the hydrophilic backbone HB, thus forming an amide function resulting from the reaction of an amine function of the precursor of the hydrophobic radical and an acid function borne by the precursor of the hydrophilic backbone HB or
   via a covalent bond between a nitrogen atom of the hydrophobic radical and a nitrogen atom of the hydrophilic backbone HB, thus forming an amide function resulting from the reaction of an acid function of the precursor of the hydrophobic radical and an amine function of the precursor of the hydrophilic backbone HB;
t is an integer equal to 0 or to 1;
B is a linear or branched alkyl radical, optionally comprising an aromatic nucleus, comprising from 1 to 9 carbon atoms or an ether radical or unsubstituted polyether comprising from 4 to 14 carbon atoms and 1 to 5 oxygen atoms;
Cₓ is a linear or branched monovalent alkyl radical, optionally comprising a cyclic part, wherein x indicates the number of carbon atoms and 11≤x≤25;
I, II″ and I [symbol] , whether they are identical or different, are divalent radicals, chosen in the group consisting of a linear or branched alkyl radical, comprising from 1 to 12 carbon atoms,
I is a trivalent radical, chosen in the group consisting of a linear or branched alkyl radical, comprising from 1 to 12 carbon atoms,
Im is an imidazolyl radical,
R is a radical chosen in the group consisting of a divalent, linear or branched alkyl radical comprising from 1 to 12 carbon atoms, a branched alkyl radical of 1 to 8 carbon atoms said alkyl radical bearing one or more free carboxylic acid function(s), a divalent, linear or branched alkyl radical comprising from 1 to 12 carbon atoms bearing one or more functions —CONH₂ or a radical ether or an unsubstituted polyether comprising from 4 to 14 carbon atoms and from 1 to 5 oxygen atoms, said free carboxylic acid functions being in the form of an alkali metal salt chosen in the group consisting of Na⁺ and K⁺, and
   when several hydrophobic radicals are borne by a hydrophilic backbone HB, then they are identical or different.

In one embodiment, the invention relates to a stable composition as defined above characterized in that the hydrophobic radical -Hy is chosen among the radicals according to formula I:

*-(GpR)ₜ-(GpI)ᵢ-[(GpR)ᵣ [symbol] (GpI)ᵢ [symbol] ]ₜ-GpC Formula I

wherein,
GpI is a divalent radical, said radical comprising at least one imidazole Im unit according to formula III:

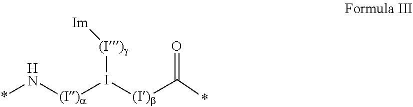

GpR is a radical according to formulas II, II [symbol] or II″:

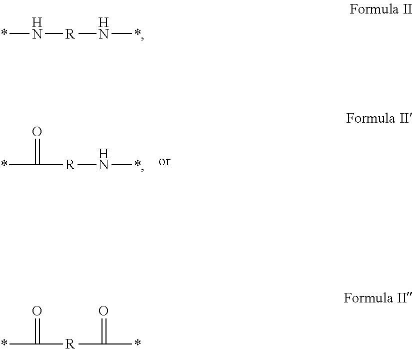

GpC is a radical according to Formula IV according to Formula IV

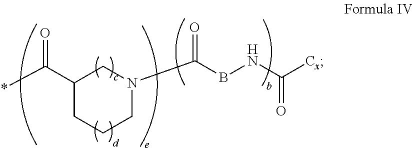

the * indicate the attachment sites of the hydrophobic radical -Hy to the hydrophilic backbone HB or the above radicals (I, II, II [symbol] , II″, III and IV) with each other via amide functions;
α, β and γ are identical or different integers equal to 0 or 1;
b is an integer equal to 0 or to 1;
c is an integer equal to 0 or 1;
d is an integer equal to 0, 1 or 2; and if c is equal to 0 then d is equal to 1 or 2;
e is an integer equal to 0 or to 1;
i and i [symbol] , whether they are identical or different, are integers less than or equal to 6 and i+i [symbol] is greater than or equal to 1 and less than or equal to 6, 1≤i+i [symbol] 6,
r and r [symbol] are integers equal to 0, 1, 2 or 3;
if r is equal to 0, then the hydrophobic radical according to formula I is bound to the hydrophilic backbone HB: via a covalent bond between a carbonyl of the hydrophobic radical and a nitrogen atom of the hydrophilic backbone HB, thus forming an amide function resulting from the reaction of an amine function of the precursor of the hydrophilic backbone HB and an acid function borne by the precursor of the hydrophobic radical, and
if r is equal to 1, 2 or 3 then the hydrophobic radical -Hy according to formula I is bound to the hydrophilic backbone HB:
   via a covalent bond between a nitrogen atom of the hydrophobic radical and a carbonyl of the hydrophilic backbone HB, thus forming an amide function resulting from the reaction of an amine function of the precursor of the hydrophobic radical and an acid function borne by the precursor of the hydrophilic backbone HB or
   via a covalent bond between a nitrogen atom of the hydrophobic radical and a nitrogen atom of the hydrophilic backbone HB, thus forming an amide function resulting from the reaction of an acid function of the precursor of the hydrophobic radical and an amine function of the precursor of the hydrophilic backbone HB;
t is an integer equal to 0 or to 1;
B is a linear or branched alkyl radical, optionally comprising an aromatic nucleus, comprising from 1 to 9 carbon atoms, or a radical ether or an unsubstituted polyether comprising from 4 to 14 carbon atoms and from 1 to 5 oxygen atoms;
Cₓ is a linear or branched monovalent alkyl radical, optionally comprising a cyclic part, wherein x indicates the number of carbon atoms and 11≤x≤25;
I [symbol] , II″ and I [symbol] , whether they are identical or different, are divalent radicals, chosen in the group consisting of a linear or branched alkyl radical, comprising from 1 to 12 carbon atoms,
I is a trivalent radical, chosen in the group consisting of a linear or branched alkyl radical, comprising from 1 to 12 carbon atoms,
Im is an imidazolyl radical,
R is a radical chosen in the group consisting of a divalent, linear or branched alkyl radical comprising from 1 to 12 carbon atoms, a divalent, linear or branched alkyl radical comprising from 1 to 12 carbon atoms bearing one or more functions —CONH₂ or a radical ether or an unsubstituted polyether comprising from 4 to 14 carbon atoms and from 1 to 5 oxygen atoms,
   when several hydrophobic radicals are borne by a hydrophilic HB backbone, then they are identical or different.

In one embodiment, the invention relates to a stable composition as defined above characterized in that the hydrophobic radical -Hy is chosen among the radicals according to formula I:

*-(GpR)ᵣ-(GpI)ᵢ[(GpR)ᵣ [symbol] (GpI)ᵢ [symbol] ]ₜ-GpC Formula I

wherein,
GpI is a divalent radical, said radical comprising at least one imidazole Im unit according to formula III:

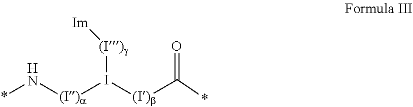

GpR is a radical according to formulas II, II [symbol] or II″:

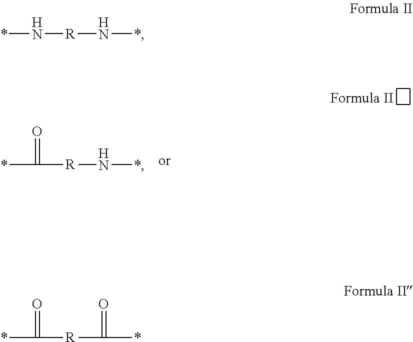

GpC is a radical according to Formula IV:

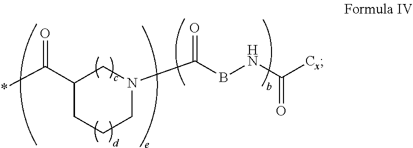

the * indicate the attachment sites of the hydrophobic radical -Hy to the hydrophilic backbone HB or the above radicals (I, II, II [symbol] , II″, III and IV) with each other via amide functions;
α, β and γ are identical or different integers equal to 0 or 1;
b is an integer equal to 0 or to 1;
c is an integer equal to 0 or 1;
d is an integer equal to 0, 1 or 2; and if c is equal to 0 then d is equal to 1 or 2;
e is an integer equal to 0 or to 1;
i and i [symbol] , whether they are identical or different, are integers less than or equal to 6 and i+i [symbol] is greater than or equal to 1 and less than or equal to 6, 1≤i+i [symbol] ≤6,
r and r [symbol] are integers equal to 0, 1, 2 or 3;
if r is equal to 0, then the hydrophobic radical according to formula I is bound to the hydrophilic backbone HB: via a covalent bond between a carbonyl of the hydrophobic radical and a nitrogen atom of the hydrophilic backbone HB, thus forming an amide function resulting from the reaction of an amine function of the precursor of the hydrophilic backbone HB and an acid function borne by the precursor of the hydrophobic radical, and
if r is equal to 1, 2 or 3 then the hydrophobic radical -Hy according to formula I is bound to the hydrophilic backbone HB:
   via a covalent bond between a nitrogen atom of the hydrophobic radical and a carbonyl of the hydrophilic backbone HB, thus forming an amide function resulting from the reaction of an amine function of the precursor of the hydrophobic radical and an acid function borne by the precursor of the hydrophilic backbone HB or
   via a covalent bond between a carbonyl of the hydrophobic radical and a nitrogen atom of the hydrophilic backbone HB, thus forming an amide function resulting from the reaction of an acid function of the precursor of the hydrophobic radical and an amine function of the precursor of the hydrophilic backbone HB;
t is an integer equal to 0 or to 1;
B is a linear or branched alkyl radical, optionally comprising an aromatic nucleus, comprising from 1 to 9 carbon atoms, or a radical ether or an unsubstituted polyether comprising from 4 to 14 carbon atoms and from 1 to 5 oxygen atoms;
Cₓ is a linear or branched monovalent alkyl radical, optionally comprising a cyclic part, wherein x indicates the number of carbon atoms and 11≤x≤25;
I [symbol] , I″ and I [symbol] , whether they are identical or different, are divalent radicals, chosen in the group consisting of a linear or branched alkyl radical, comprising from 1 to 12 carbon atoms,
I is a trivalent radical, chosen in the group consisting of a linear or branched alkyl radical, comprising from 1 to 12 carbon atoms,
Im is an imidazolyl radical,
R is a radical chosen in the group consisting of a branched alkyl radical of from 1 to 8 carbon atoms, said alkyl radical bearing one or more free carboxylic acid function(s), said free carboxylic acid functions being in the form of an alkali metal salt chosen in the group consisting of Na⁺ and K⁺, and
   when several hydrophobic radicals are borne by a hydrophilic HB backbone, then they are identical or different.

In one embodiment, the composition according to the invention is characterized in that Hy comprises from 15 to 100 carbon atoms.

In one embodiment, the composition according to the invention is characterized in that Hy comprises from 30 to 70 carbon atoms.

In one embodiment, the composition according to the invention is characterized in that Hy comprises from 40 to 60 carbon atoms.

In one embodiment, the composition according to the invention is characterized in that Hy comprises from 40 to 50 carbon atoms.

In one embodiment, the composition according to the invention is characterized in that Hy comprises from 50 to 60 carbon atoms.

In one embodiment, the composition according to the invention is characterized in that Hy comprises from 20 to 40 carbon atoms.

In one embodiment, the composition according to the invention is characterized in that Hy comprises from 20 to 30 carbon atoms.

In one embodiment, the composition according to the invention is characterized in that Hy comprises from 30 to 40 carbon atoms.

In one embodiment, Hy comprises more than 15 carbon atoms.

In one embodiment, Hy comprises more than 30 carbon atoms.

In one embodiment, the composition is characterized in that the pH is from 6.0 to 8.0.

In one embodiment, the composition is characterized in that the pH is from 6.6 to 7.8.

In one embodiment, the composition is characterized in that the pH is from 7.0 to 7.8.

In one embodiment, the composition is characterized in that the pH is from 6.8 to 7.4.

In one embodiment, when r=2, then the GpR group linked to the hydrophilic backbone HB is chosen among the GpRs according to formula II.

In one embodiment, when r=2, then the GpR group linked to the hydrophilic backbone HB is chosen among the GpRs according to formula II and the second GpR is chosen among the GpRs according to formula II″.

In one embodiment, an embodiment, when r=2, then the GpR group linked to the hydrophilic backbone HB is chosen among the GpRs according to formula II″.

In one embodiment, an embodiment, when r=2, then the GpR group linked to the hydrophilic backbone HB is chosen among the GpRs according to formula II″ and the second GpR is chosen among the GpRs according to formula II.

In one embodiment, said at least one hydrophobic radical [symbol] Hy is chosen among according to formula I wherein, t=0, r [symbol] 0 and i [symbol] =0 according to formula Ia, as defined below:

*-(GpR)ᵣ-(GpI)ᵢ-GpC Formula Ia

wherein GpR, GpI, GpC, r and i have the definitions given above.

In one embodiment, said at least one hydrophobic radical [symbol] Hy is chosen among according to formula I wherein r=2 , r [symbol] 0 and i [symbol] 0 according to formula Ib, as defined below:

*-GpR₁-GpR-(GpI)ᵢ-GpC Formula Ib

wherein GpR₁ is aradical according to formula II,

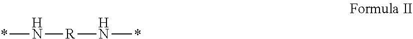

wherein GpR, GpA, GpC, R, and i have the definitions given above.

In one embodiment, said at least one hydrophobic radical [symbol] Hy is chosen among according to formula I wherein r=2, r [symbol] 0 and i [symbol] 0 according to formula Ib as defined below:

*-GpR₁-GpR-(GpI)ᵢGpC Formula Ib

wherein GpR₁ is a radical according to formula II

[symbol]

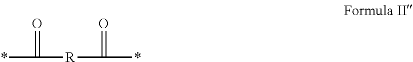

wherein GpR, GpI, GpI, GpC, R, and i have the definitions given above.

In one embodiment, said at least one hydrophobic radical [symbol] Hy is chosen among radicals according to formula I wherein r=1 , r [symbol] 0 and i [symbol] 0 according to formula Ic, as defined below:

*-GpR-(GpI)ᵢGpC Formula Ic

wherein GpR is a radical according to formula II

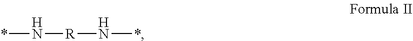

wherein GpR, GpI, GpC, R, and i have the definitions given above.

In one embodiment, said at least one hydrophobic radical [symbol] Hy is chosen among according to formula I wherein r=1 , r [symbol] 0 and i [symbol] 0 according to formula Ic, as defined below:

*-GpR-(GpI)ᵢ GpC Formula Ic

wherein GpR is a radical according to formula II

[symbol]

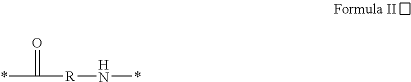

wherein GpR, GpI, GpC, R, and i have the definitions given above.

In one embodiment, said at least one hydrophobic radical [symbol] Hy is chosen among radicals according to formula I wherein r=1, r [symbol] 0 and i [symbol] 0 according to formula Ic, as defined below:

*-GpR-(GpI)ᵢ GpC Formula Ic

wherein GpR is a radical according to formula II

[symbol]

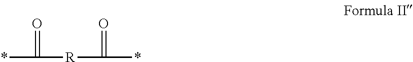

wherein GpR, GpC, GpI, R, and i have the definitions given above.

In one embodiment, said at least one hydrophobic radical [symbol] Hy is chosen among according to formula I wherein i=1, r [symbol] 0 and i [symbol] 0 according to formula Id, as defined below:

*-(GpR)ᵣ-GpI-GpC Formula Id

wherein GpR, GpC, GpI, and r have the definitions given above.

In one embodiment, said at least one hydrophobic radical [symbol] Hy is chosen among formularadicals according to formula I wherein i=3, r [symbol] 0 and i [symbol] 0 according to formula Ie, as defined below:

*(GpR)ᵣ-(GpI)₃-GpC Formula Ie

wherein GpR, GpI, GpC, and r have the definitions given above.

In one embodiment, said at least one hydrophobic radical [symbol] Hy is chosen among radicals according to formula I wherein r=0, r [symbol] 0 and i [symbol] 0 according to formula If, as defined below:

*-(GpI)ᵢ-GpC Formula If

wherein GpI, GpC, and i have the definitions given above.

According to one particular embodiment i=1.

According to one particular embodiment i=2.

According to one particular embodiment i=3.

In one embodiment, said at least one hydrophobic radical [symbol] Hy is chosen among the radicals according to formula I, Ia, Ib, Ic, Id, Ie or If wherein theradical according to formula III is chosen among the radicals according to formula Ma:

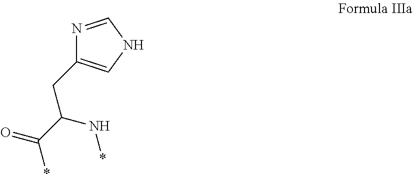 [SMILES: *NC(Cc1c[nH]cn1)C(*)=O]

In one embodiment, said at least one hydrophobic radical [symbol] Hy is chosen among the radicals according to formula I, Ia, Ib, Ic, Id, Ie or If wherein the radical according to formula III is chosen among the radicals according to formula IIIb:

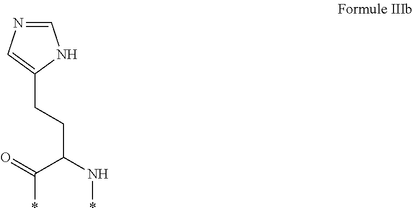 [SMILES: *NC(CCc1cnc[nH]1)C(*)=O]

In one embodiment, said at least one hydrophobic radical [symbol] Hy is chosen among the radicals according to formula I, Ia, Ib, Ic, Id, Ie or If wherein theradical according to formula III is chosen among the radicals according to formula IIIc:

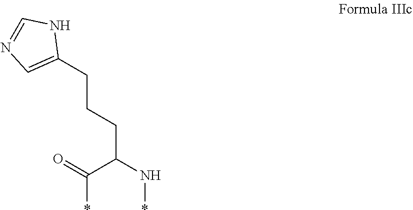 [SMILES: *NC(CCCc1cnc[nH]1)C(*)=O]

In one embodiment, said at least one hydrophobic radical [symbol] Hy is chosen among the radicals according to formula I, Ia, Ib, Ic, Id, Ie or If wherein theradical according to formula III is chosen among the radicals according to formula IIId:

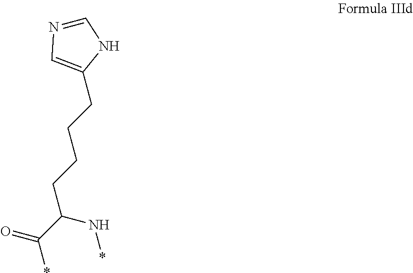 [SMILES: *NC(CCCCc1cnc[nH]1)C(*)=O]

In one embodiment, said at least one hydrophobic radical [symbol] Hy is chosen among the radicals according to formula I, Ia, Ib, Ic, Id, Ie or If wherein the radical according to formula III is chosen among the radicals according to formula IIIe:

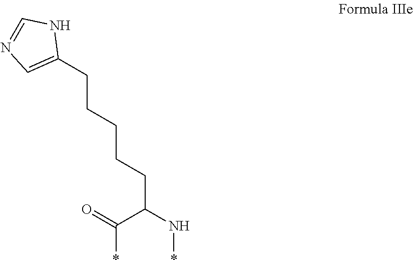 [SMILES: *NC(CCCCCc1cnc[nH]1)C(*)=O]

In one embodiment, said at least one hydrophobic radical [symbol] Hy is chosen among the radicals according to formula I, Ia, Ib, Ic, Id, Ie or If wherein the radical according to formula III is chosen among the radicals according to formulas IIIf:

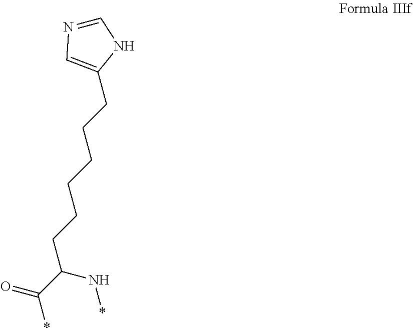 [SMILES: *NC(CCCCCCc1cnc[nH]1)C(*)=O]

In one embodiment, said at least one hydrophobic radical [symbol] Hy is chosen among the radicals according to formula I, Ia, Ib, Ic, Id, Ie or If wherein the radical according to formula III is chosen among the radicals according to formula IIIg:

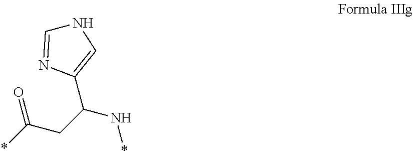 [SMILES: *NC(CC(*)=O)c1c[nH]cn1]

In one embodiment, said at least one hydrophobic radical [symbol] Hy is chosen among the radicals according to formula I, Ia, Ib, Ic, Id, Ie or If wherein the radical according to formula III is chosen among the radicals according to formula IIIh:

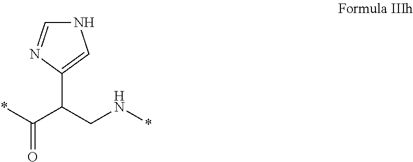 [SMILES: *NCC(C(*)=O)c1c[nH]cn1]

In one embodiment, said at least one hydrophobic radical [symbol] Hy is chosen among the radicals according to formula I, Ia, Ib, Ic, Id, Ie or If wherein the radical according to formula III is chosen among the radicals according to formulas IIIi:

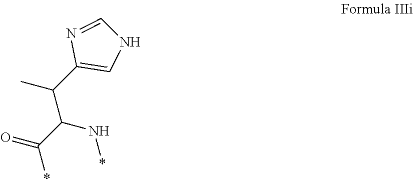 [SMILES: *NC(C(*)=O)C(C)c1c[nH]cn1]

In one embodiment, said at least one hydrophobic radical [symbol] Hy is chosen among the radicals according to formula I, Ia, Ib, Ic, Id, Ie or If wherein the precursor of the radical according to formula III is chosen among histidine and its isomers, the CAS of which are (L): 71-00-1, (D): 351-50-8, (racemic): 4998-57-6) 2-amino-4-(1H-imidazol-5-yl) butanoic acid and its isomers whose CAS are (racemic): 5817-77-6, (S): 58-501-47-6, (R) 58501-48-7, 2-amino-5-(1H-imidazol-5-yl) pentanoic acid and its isomers whose CAS are (racemic): 916050-51-6, (S): 250578-07-5), 2-amino-6-(1H-imidazol-5-yl) hexanoic acid and its isomers whose CAS are (racemic): 2167109-48-8), (S): 250578-08-6), 2-amino-7-(1H-imidazol-5-yl) hexanoic acid and its isomers whose CAS are (racemic): 2168144-96-3, (S): 250578-09-7), 2-amino-8-(1H-imidazol-5-yl) octanoic acid and its isomers whose CAS are: 2167137-07-5, (S): 250578-10-0), 1H-imidazole-4-propanoic acid, beta-amino- and its isomers whose CAS are (racemic): 207674-08-6, (S): 1062610-63-2, (R): 1062610-66-5, 1H-Imidazole-4-aceticacid,alpha-(aminomethyl) whose CAS is: 757185-97-0) and β-Methylhistidine and its isomers whose CAS are (racemic, racemic): 26798-08-3, (S,S): 215932-30-2, (R,S): 215932-31-3, (S,R): 215932-33-4, (R,R): 215932-33-5, (racemic, S): 1933687-26-3.

In one embodiment, the composition is characterized in that the hydrophobic radical according to formula I, Ia, Ib, Ie, Id or Ie wherein GpI is a radical according to formula III, wherein α=0, β=0, γ=1, I is a —CH— group and I [symbol] is a radical alkyl comprising 1 carbon atom represented by —CH₂—.

In one embodiment, the composition is characterized in that the hydrophobic radical according to formula I, Ia, Ib, Ic, Id, Ie or If wherein GpI is a radical according to formula III, wherein α=0, β=0, γ=1, I is a —CH— group and I [symbol] is a radical alkyl comprising 2 carbon atoms represented by —CH₂—CH₂—

In one embodiment, the composition is characterized in that the hydrophobic radical according to formula I, Ia, Ib, Ic, Id, Ie or If wherein GpI is a radical according to formula III, wherein α=0, β=0, γ=1, I is a —CH— group and I [symbol] is a radical alkyl comprising 3 carbon atom represented by [symbol] (CH₂)₃—.

In one embodiment, the composition is characterized in that the hydrophobic radical according to formula I, Ia, Ib, Ic, Id, Ie or If wherein GpI is a radical according to formula III, wherein α=0, β=0, γ=1, I is a —CH— group and I [symbol] is a radical alkyl comprising 3 carbon atoms represented by —(CH₂)₄—.

In one embodiment, the composition is characterized in that the hydrophobic radical according to formula I, Ia, Ib, Ic, Id, Ie or If wherein GpI is a radical according to formula III, wherein α=0, β=0, γ=1, I is a —CH— group and I [symbol] is a radical alkyl comprising 3 carbon atoms represented by —(CH₂)₅—.

In one embodiment, the composition is characterized in that the hydrophobic radical according to formula I, Ia, Ib, Ic, Id, Ie or If wherein GpI is a radical according to formula III, wherein α=0, β=0, γ=1, I is a —CH— group and I [symbol] is a radical alkyl comprising 3 carbon atom represented by [symbol] (CH₂)₆—.

In one embodiment, the composition is characterized in that the hydrophobic radical according to formula I, Ia, Ib, Ic, Id, Ie or If wherein GpI is a radical according to formula III, wherein α=0, β=0, γ=1, I is a —CH— group and I [symbol] is a —CH₂— group.

In one embodiment, the composition is characterized in that the hydrophobic radical according to formula I, Ia, Ib, Ic, Id, Ie or If wherein GpI is a radical according to formula III, wherein α=0, β=0, γ=1, I is a —CH— group and I″ is a —CH₂— group.

In one embodiment, the composition is characterized in that the hydrophobic radical according to formula I, Ia, Ib, Ic, Id, Ie or If wherein GpI is a radical according to formula III, wherein α=0, β=0, γ=1, I is a —CH— group and I″ [symbol] is a —(CH)—CH₃ group.

In one embodiment, said at least one hydrophobic radical [symbol] Hy is chosen among the radicals according to formula I, Ia, Ib, Ic, Id, Ie or If wherein GpC is a radical according to Formula IV wherein e=0, and GpC is a radical according to Formula IVa.

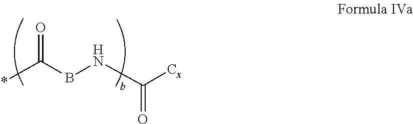

wherein B, b and Cx have the definitions given above.

In one embodiment, said at least one hydrophobic radical [symbol] Hy is chosen among the radicals according to formula I, Ia, Ib, Ic, Id, Ie or If wherein GpC is a radical according to Formula IV wherein e=1 and GpC is a radical according to Formula IVb.

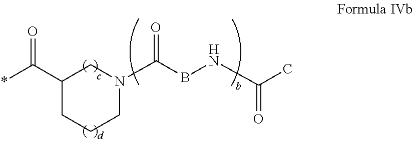

wherein c, d, B, b and Cx have the definitions given above.

In one embodiment, said at least one hydrophobic radical [symbol] Hy is chosen among the radicals according to formula I, Ia, Ib, Ic, Id, Ie or If wherein GpC is a radical according to Formula IV wherein e=1 b=0 and GpC is a radical according to Formula IVc.

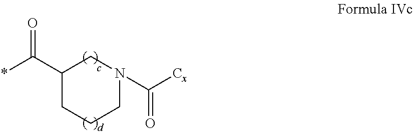

wherein c, d and Cx have the definitions given above.

In one embodiment, said at least one hydrophobic radical [symbol] Hy is chosen among the radicals according to formula I, Ia, Ib, Ic, Id, Ie or If wherein GpC is a radical according to Formula IV wherein e=0 b=0 and GpC is a radical according to Formula IVd.

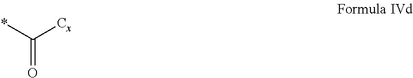

where Cx has the definition given above.

In one embodiment, the composition is characterized in that the hydrophobic radical is a radical according to formula I, Ia, Ib, Ic, Id or Ie.

In one embodiment, the composition is characterized in that the hydrophobic radical is a radical according to formula I, Ia, Ib, Ic, Id or Ie wherein GpR is a radical according to formula II wherein R is a linear divalent alkyl radical comprising from 2 to 12 carbon atoms.

In one embodiment, the composition is characterized in that the hydrophobic radical is a radical according to formula I, Ia, Ib, Ic, Id or Ie wherein GpR is a radical according to formula II wherein R is a linear divalent alkyl radical comprising from 2 to 6 carbon atoms.

In one embodiment, the composition is characterized in that the hydrophobic radical is a radical according to formula I, Ia, Ib, Ic, Id or Ie wherein GpR is a radical according to formula II wherein R is a linear divalent alkyl radical comprising from 2 to 6 carbon atoms.

In one embodiment, the composition is characterized in that the hydrophobic radical is a radical according to formula I, Ia, Ib, Ic, Id or Ie wherein GpR is a radical according to formula II wherein R is a linear divalent alkyl radical comprising from 2 to 4 carbon atoms.

In one embodiment, the composition is characterized in that the hydrophobic radical is a radical according to formula I, Ia, Ib, Ic, Id or Ie wherein GpR is a radical according to formula II wherein R is a linear divalent alkyl radical comprising from 2 to 4 carbon atoms.

In one embodiment, the composition is characterized in that the hydrophobic radical is a radical according to formula I, Ia, Ib, Ic, Id or Ie wherein GpR is a radical according to formula II wherein R is a linear divalent alkyl radical comprising from 2 carbon atoms.

In one embodiment, the composition is characterized in that the hydrophobic radical is a radical according to formula I, Ia, Ib, Ic, Id or Ie wherein GpR is a radical according to Formula II [symbol] .

In one embodiment, the composition is characterized in that the hydrophobic radical is a radical according to formula I, Ia, Ib, Ic, Id or Ie wherein GpR is a radical according to Formula II [symbol] wherein R is a divalent linear alkyl radical comprising from 2 to 12 carbon atoms.

In one embodiment, the composition is characterized in that the hydrophobic radical is a radical according to formula I, Ia, Ib, Ic, Id or Ie wherein GpR is a radical according to Formula II [symbol] wherein R is a linear divalent alkyl radical comprising from 2 to 6 carbon atoms.

In one embodiment, the composition is characterized in that the hydrophobic radical is a radical according to formula I, Ia, Ib, Ic, Id or Ie wherein GpR is a radical according to Formula II [symbol] wherein R is a linear divalent alkyl radical comprising from 2 to 6 carbon atoms.

In one embodiment, the composition is characterized in that the hydrophobic radical is a radical according to formula I, Ia, Ib, Ic, Id or Ie wherein GpR is a radical according to Formula II [symbol] wherein R is a linear divalent alkyl radical comprising from 2 to 4 carbon atoms.

In one embodiment, the composition is characterized in that the hydrophobic radical is a radical according to formula I, Ia, Ib, Ic, Id or Ie wherein GpR is a radical according to Formula II [symbol] wherein R is a linear divalent alkyl radical comprising from 2 to 4 carbon atoms.

In one embodiment, the composition is characterized in that the hydrophobic radical is a radical according to formula I, Ia, Ib, Ic, Id or Ie wherein GpR is a radical according to Formula II [symbol] wherein R is a linear divalent alkyl radical comprising from 2 carbon atoms.

In one embodiment, the composition is characterized in that the hydrophobic radical is a radical according to formula I, Ia, Ib, Ic, Id or Ie wherein GpR is a radical according to Formula II″.

In one embodiment, the composition is characterized in that the hydrophobic radical is a radical according to formula I, Ia, Ib, Ic, Id or Ie wherein GpR is a radical according to Formula II″ wherein R is a linear divalent alkyl radical comprising from 2 to 12 carbon atoms.

In one embodiment, the composition is characterized in that the hydrophobic radical is a radical according to formula I, Ia, Ib, Ic, Id or Ie wherein GpR is a radical according to Formula II″ wherein R is a linear divalent alkyl radical comprising from 2 to 6 carbon atoms.

In one embodiment, the composition is characterized in that the hydrophobic radical is a radical according to formula I, Ia, Ib, Ic, Id or Ie wherein GpR is a radical according to Formula II″ wherein R is a linear divalent alkyl radical comprising from 2 to 6 carbon atoms.

In one embodiment, the composition is characterized in that the hydrophobic radical is a radical according to formula I, Ia, Ib, Ic, Id or Ie wherein GpR is a radical according to Formula II″ wherein R is a linear divalent alkyl radical comprising from 2 to 4 carbon atoms.

In one embodiment, the composition is characterized in that the hydrophobic radical is a radical according to formula I, Ia, Ib, Ic, Id or Ie wherein GpR is a radical according to Formula II″ wherein R is a linear divalent alkyl radical comprising from 2 to 4 carbon atoms.

In one embodiment, the composition is characterized in that the hydrophobic radical is a radical according to formula I, Ia, Ib, Ic, Id or Ie wherein GpR is a radical according to Formula II″ wherein R is a linear divalent alkyl radical comprising from 2 carbon atoms.

In one embodiment, the composition is characterized in that the hydrophobic radical is a radical according to formula I, Ia, Ib, Ic, Id or Ie wherein GpR is a radical according to formula II, Formula II [symbol] , Formula II″, wherein R is a linear unsubstituted ether or polyether radical comprising from 4 to 14 carbon atoms and from 1 to 5 oxygen atoms.

In one embodiment, the composition is characterized in that the hydrophobic radical is a radical according to formula I, Ia, Ib, Ic, Id or Ie wherein GpR is a radical according to formula II, II [symbol] , II″, wherein R is an ether radical,

In one embodiment, the composition is characterized in that the hydrophobic radical is a radical according to formula I, Ia, Ib, Ic, Id or Ie wherein GpR is a radical according to formula II, II [symbol] , II″, wherein R is an ether radical comprising from 4 to 6 carbon atoms.

In one embodiment, the composition is characterized in that the hydrophobic radical is a radical according to formula I, Ia, Ib, Ic, Id or Ie wherein GpR is a radical according to formula II, II [symbol] II″ wherein R is an ether radical represented by the formula

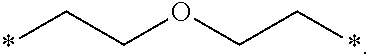

In one embodiment, the composition is characterized in that the hydrophobic radical is a radical according to formula I, Ia, Ib, Ic, Id or Ie wherein GpR is a radical according to formula II or II [symbol] , wherein R is a polyether radical,

In one embodiment, the composition is characterized in that the hydrophobic radical is a radical according to formula I, Ia, Ib, Ic, Id or Ie wherein GpR is a radical according to formula II or II [symbol] , wherein R is a linear polyether radical comprising from 6 to 10 carbon atoms. and from 2 to 3 oxygen atoms.

In one embodiment, the composition is characterized in that the hydrophobic radical according to formula I, Ia, Ib, Ic Id or Ie wherein GpR is a radical according to formula II or II [symbol] , wherein R is a polyether radical chosen in the group consisting of the radicals represented by the formulas below:

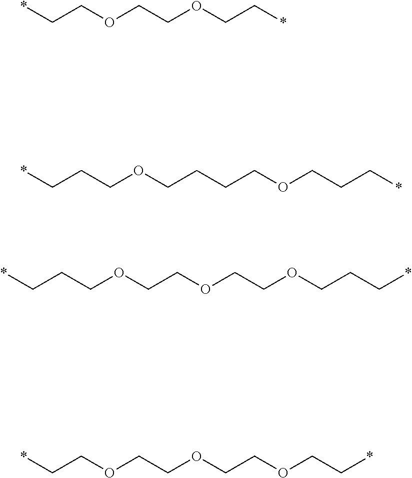 [SMILES: *CCOCCOCC* ; *CCCOCCCCOCCC* ; *CCCOCCOCCOCCC* ; *CCOCCOCCOCC*]

In one embodiment, the composition is characterized in that the hydrophobic radical according to formula I, Ia, Ib, Ie Id or Ie wherein GpR is a radical according to formula II wherein R is a polyether radical chosen in the group consisting of the radicals represented by the formulas below:

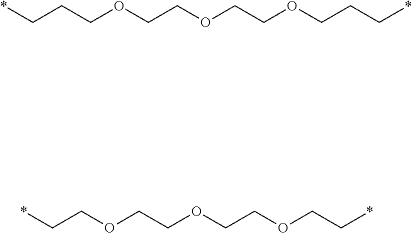 [SMILES: *CCCOCCOCCOCCC* ; *CCOCCOCCOCC*]

In one embodiment, the composition is characterized in that the hydrophobic radical is a radical according to formula I, Ia, Ib, Ic, or Ie, GpR is chosen among formulas II, II [symbol] and/or II″ and i=1.

In one embodiment, the composition is characterized in that the hydrophobic radical is a radical according to formula I, Ia, Ib, Ic or Ie wherein GpR is a radical according to formula II and i=1.

In one embodiment, the composition is characterized in that the hydrophobic radical is a radical according to formula I, Ia, Ib, Ic or Ie wherein GpR is a radical according to formula II″ and i=1.

In one embodiment, the composition is characterized in that the hydrophobic radical is a radical according to formula I, Ia, Ib, or Ie, GpR is chosen among formulas II, II [symbol] and/or II″ and i=2

In one embodiment, the composition is characterized in that the hydrophobic radical is a radical according to formula I, Ia, Ib, or Ie wherein GpR is radical according to formula II and i=2.

In one embodiment, the composition is characterized in that the hydrophobic radical is a radical according to formula I, Ia, Ib, or Ie wherein GpR is a radical according to formula II″ and i=2.

In one embodiment, the composition is characterized in that the hydrophobic radical is a radical according to formula I, Ia, Ib, Id or Ie, GpR is chosen among formulas II, II [symbol] and/or II″ and i=3

In one embodiment, the composition is characterized in that the hydrophobic radical is a radical according to formula I, Ia, Ib, Id or Ie wherein GpR is a radical according to formula II and i=3.

In one embodiment, the composition is characterized in that the hydrophobic radical is a radical according to formula I, Ia, Ib, Id or Ie wherein GpR is a radical according to formula II″ and i=3.

In one embodiment, the composition is characterized in that the hydrophobic radical is a radical according to formula I, Ia, Ib, Ic, Id or Ie wherein GpR is a radical according to formula II, II [symbol] , II″ wherein R represents a branched alkyl radical comprising from 1 to 8 carbon atoms, said alkyl radical bearing one or more free carboxylic acid function(s).

In one embodiment, the composition is characterized in that the hydrophobic radical is a radical according to formula I, Ia, Ib, Ic, Id or Ie wherein GpR is a radical according to formula II, wherein R represents a branched alkyl radical comprising from 1 to 8 carbon atoms said alkyl radical bearing one or more free carboxylic acid function(s).

In one embodiment, the composition is characterized in that the hydrophobic radical is a radical according to formula I, Ia, Ib, Ic, Id or Ie wherein GpR is a radical according to formula II [symbol] wherein R represents a branched alkyl radical comprising from 1 to 8 carbon atoms said alkyl radical bearing one or more free carboxylic acid function(s).

In one embodiment, the composition is characterized in that the hydrophobic radical is a radical according to formula I, Ia, Ib, Ic, Id or Ie wherein GpR is a radical according to II″ wherein R is a branched alkyl radical comprisingfrom 1 to 8 carbon atoms, said alkyl radical bearing one or more free carboxylic acid function(s).

In one embodiment, the composition is characterized in that the hydrophobic radical is a radical according to formula I, Ia, Ib, Ic, Id or Ie wherein GpR is a radical according to formula II, II [symbol] , II″ wherein R is a branched alkyl radical comprising from 1 to 6 carbon atoms, said alkyl radical bearing one free carboxylic acid function.

In one embodiment, the composition is characterized in that the hydrophobic radical is a radical according to formula I, Ia, Ib, Ic, Id or Ie wherein GpR is a radical according to formula II wherein R represents a branched alkyl radical comprising from 1 to 6 carbon atoms, said alkyl radical bearing a free carboxylic acid function.

In one embodiment, the composition is characterized in that the hydrophobic radical is a radical according to formula I, Ia, Ib, Ic, Id or Ie wherein GpR is a radical according to Formula II [symbol] wherein R represents a branched alkyl radical comprising from 1 to 6 carbon atoms. said alkyl radical bearing a free carboxylic acid function.

In one embodiment, the composition is characterized in that the hydrophobic radical is a radical according to formula I, Ia, Ib, Ic, Id or Ie wherein GpR is a radical according to Formula II″ wherein R is a branched alkyl radical comprising from 1 to 6 carbon atoms, said alkyl radical bearing one free carboxylic acid function.

In one embodiment, the composition is characterized in that the hydrophobic radical according to formula I, Ia, Ib, Ic Id or Ie wherein GpR is a radical according to formula II, wherein R is an alkyl radical comprising from 5 carbon atoms and bearing a free carboxylic acid function represented by formula Z below:

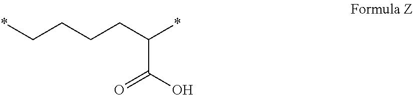 [SMILES: *CCCCC(*)C(=O)O]

In one embodiment, the composition is characterized in that the hydrophobic radical according to formula I, Ia, Ib, Ic Id or Ie wherein GpR is a radical according to formula II wherein R is a radical according to Formula Z whose precursor is lysine.

In one embodiment, the composition is characterized in that the hydrophobic radical according to formula I, Ia, Ib, Ic Id or Ie wherein GpR is a radical according to Formula II [symbol] , wherein R is an alkyl radical comprising 3 carbon atoms and bearing a free carboxylic acid function represented by formula Z′ below:

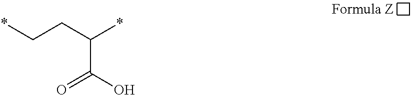 [SMILES: *CCC(*)C(=O)O]

In one embodiment, the composition is characterized in that the hydrophobic radical according to formula I, Ia, Ib, Ie Id or Ie wherein GpR is a radical according to Formula II [symbol] wherein R is a radical according to formula Z [symbol] whose precursor is glutamic acid.

In one embodiment, the composition is characterized in that the hydrophobic radical according to formula I, Ia, Ib, Ie Id or Ie wherein GpR is a radical according to formula II [symbol] , wherein R is an alkyl radical comprising 2 carbon atoms and bearing a free carboxylic acid function represented by formula Z″ below:

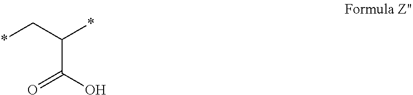 [SMILES: *CC(*)C(=O)O]

In one embodiment, the composition is characterized in that the hydrophobic radical according to formula I, Ia, Ib, Ic Id or Ie wherein GpR is a radical according to formula II [symbol] wherein R is a radical according to formula Z″ whose precursor is aspartic acid.

In one embodiment, the composition is characterized in that the hydrophobic radical according to formula I, Ia, Ib, Ic Id or Ie wherein GpR is a radical according to formula II, [symbol] , II″, wherein R is an alkyl radical comprising 5 carbon atoms represented by —(CH₂)₄—CH(COOH)—.

In one embodiment, the composition is characterized in that the hydrophobic radical according to formula I, Ia, Ib, Ic Id or Ie wherein GpR is a radical according to formula II, II [symbol] , II″, wherein R is an alkyl radical comprising 3 carbon atoms represented by —(CH₂)₂—CH(COOH)—.

In one embodiment, the composition is characterized in that the hydrophobic radical according to formula I, Ia, Ib, Ic Id or Ie wherein GpR is a radical according to formula II, II [symbol] II″, wherein R is an alkyl radical comprising 2 carbon atoms represented by —CH₂—CH(COOH).

In one embodiment, the composition is characterized in that the hydrophobic radical is a radical according to formula I, Ia, Ib, Ic or Ie wherein GpR is aradical according to formula II, i=1. and GpI is a radical according to formula IIIa.

In one embodiment, the composition is characterized in that the hydrophobic radical is a radical according to formula I, Ia, Ib, Id or Ie wherein GpR is radical according to formula II, i=3 and GpI is a radical according to formula IIIa.

In one embodiment, the composition is characterized in that the hydrophobic radical is a radical according to formula I, Ia, Ib, Ic, Id, Ie or If wherein the radical GpCaccording to formula IV chosen in the group consisting of radicals according to formulas IVe, IVf or IVg represented below:

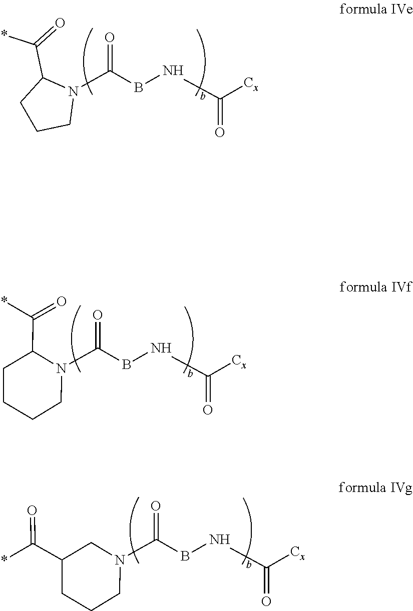

In one embodiment, the composition is characterized in that the hydrophobic radical is a radical according to formula I, Ia, Ib, Ic, Id, Ie or If wherein the GpC radical is a radical according to formula IVe.

In one embodiment, the composition is characterized in that the hydrophobic radical is a radical according to formula I, Ia, Ib, Ic, Id, Ie or If wherein GpC radical is aaccording to formula IV chosen in the group consisting of radicals according to formulas IVe, IVf or IVg wherein b is equal to 0, corresponding respectively to a radical according to formulas IVh, IVi, and IVj represented below:

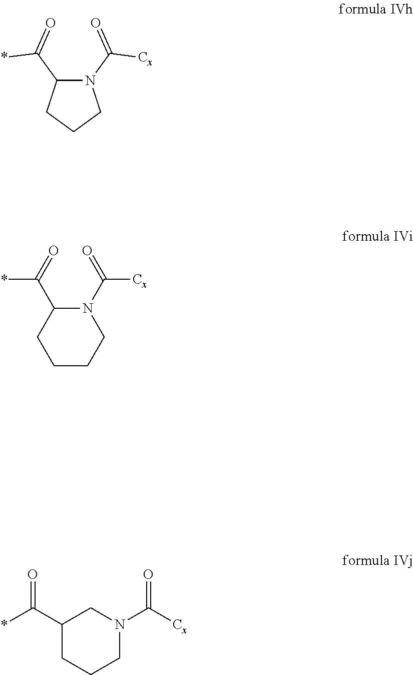

In one embodiment, the composition is characterized in that the hydrophobic radical is a radical according to formula I, Ia, Ib, Ic, Id, Ie or If wherein GpC is a radical according to formula IV or IVe wherein b=0 and corresponds to a radical according to formula IVh.

In one embodiment, the composition is characterized in that the hydrophobic radical is a radical according to formula I, Ia, Ib, Ic, Id, Ie or If wherein GpC is a radical according to formula IV wherein b=1 chosen in the group consisting of radicals wherein B is an amino acid residue chosen in the group consisting of the radicals represented by a radical according to formulas below:

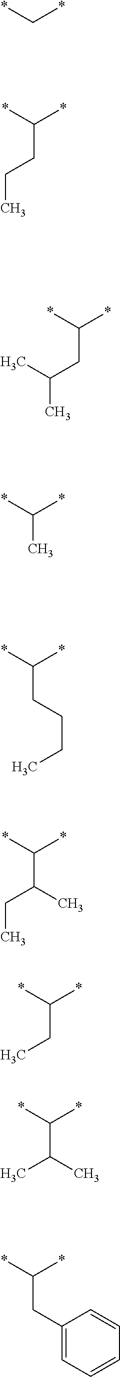

In one embodiment, the composition is characterized in that the hydrophobic radical is a radical according to formula I, Ia, Ib, Ic, Id, Ie or If wherein GpC is a radical according to formula IV chosen in the group consisting of radicals wherein Cx is chosen in the group consisting of linear alkyl radicals.

In one embodiment, the composition is characterized in that the hydrophobic radical is a radical according to formula I, Ia, Ib, Ic, Id, Ie or If wherein GpC is a radical according to formula IV is chosen in the group consisting of radicals wherein Cx is chosen in the group consisting of branched alkyl radicals.

In one embodiment, the composition is characterized in that the hydrophobic radical is a radical according to formula I, Ia, Ib, Ic, Id, Ie or If wherein GpC is a radical according to formula IV is chosen in the group consisting of radicals wherein Cx is chosen in the group consisting of alkyl radicals comprising from 11 to 14 carbon atoms.

In one embodiment, the composition is characterized in that the hydrophobic radical is a radical according to formula I, Ia, Ib, Ic, Id, Ie or If wherein GpC is a radical according to formula IV is chosen in the group consisting of radicals wherein Cx is chosen in the group consisting of the radicals represented by the formulas below:

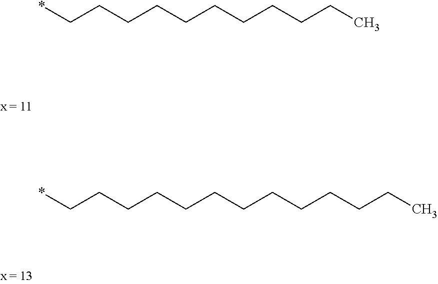 [SMILES: *CCCCCCCCCCC ; *CCCCCCCCCCCCC]

In one embodiment, the composition is characterized in that the hydrophobic radical is a radical according to formula I, Ia, Ib, Ic, Id, Ie or If wherein GpC is a radical according to formula IV is chosen in the group consisting of radicals wherein Cx is chosen in the group consisting of alkyl radicals comprising from 15 to 16 carbon atoms.

In one embodiment, the composition is characterized in that the hydrophobic radical is a radical according to formula I, Ia, Ib, Ic, Id, Ie or If wherein GpC is a radical according to formula IV is chosen in the group consisting of radicals wherein Cx is chosen in the group consisting of the radicals represented by the formulas below:

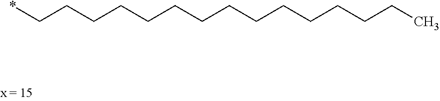 [SMILES: *CCCCCCCCCCCCCCC]

In one embodiment, the composition is characterized in that the hydrophobic radical is a radical according to formula I, Ia, Ib, Ic, Id, Ie or If wherein GpC is a radical according to formula IV is chosen amongchosen in the group consisting of radicals wherein Cx is chosen amongchosen in the group consisting of the radicals represented by the formulas below:

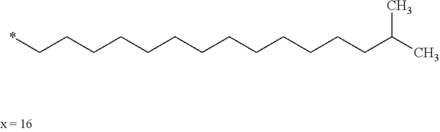 [SMILES: *CCCCCCCCCCCCCC(C)C]

In one embodiment, the composition is characterized in that the hydrophobic radical is a radical according to formula I, Ia, Ib, Ic, Id, Ie or If wherein GpC is a radical according to formula IV GpC radical is chosen in the group consisting of radicals wherein Cx is chosen in the group consisting of alkyl radicals comprising from 17 to 25 carbon atoms.

In one embodiment, the composition is characterized in that the hydrophobic radical is a radical according to formula I, Ia, Ib, Ic, Id, Ie or If wherein GpC is a radical according to formula IV GpC radical is chosen in the group consisting of radicals wherein Cx is chosen in the group consisting of alkyl radicals comprising from 17 to 18 carbon atoms.

In one embodiment, the composition is characterized in that the hydrophobic radical is a radical according to formula I, Ia, Ib, Ic, Id, Ie or If wherein GpC is a radical according to formula IV GpC radical is chosen in the group consisting of radicals wherein Cx is chosen in the group consisting of the radical alkyls represented by the formulas below:

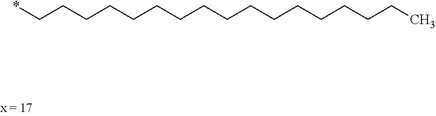 [SMILES: *CCCCCCCCCCCCCCCCC]

In one embodiment, the composition is characterized in that the hydrophobic radical is a radical according to formula I, Ia, Ib, Ic, Id, Ie or If wherein GpC is a radical according to formula IV GpC radical is gchosen in the group consisting of radicals wherein Cx is chosen in the group consisting of alkyl radicals comprising from 18 to 25 carbon atoms.

In one embodiment, the composition is characterized in that the hydrophobic radical is a radical according to formula I, Ia, Ib, Ic, Id, Ie or If wherein GpC radical according to formula IV GpC radical is chosen in the group consisting of radicals wherein Cx is chosen in the group consisting of alkyl radicals represented by the formulas below:

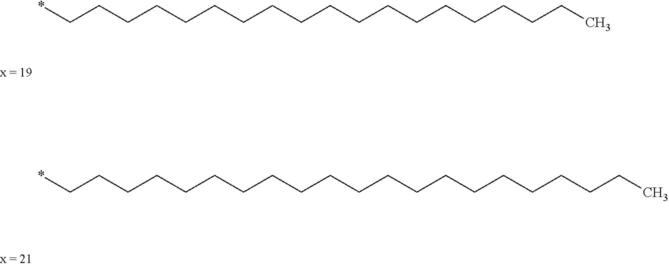 [SMILES: *CCCCCCCCCCCCCCCCCCC ; *CCCCCCCCCCCCCCCCCCCCC]

In one embodiment, the composition is characterized in that the hydrophobic radical is a radical according to formula I, Ia, Ib, Ic, Id or Ie wherein GpR is a radical according to formula II, R is a radical according to formula Z, GpI is a radical according to formula IIIa and GpC is a radical according to formula IVh.

In one embodiment, the composition is characterized in that the hydrophobic radical is a radical according to formula I, Ia, Ib, Ic or Id wherein r=1, GpR is a radical according to formula II, R is a radical according to formula Z, i=1 or 2, GpI₁ and GpI₂, which are identical, are radicals according to formula Formula IIIa, and GpC is a radical according to formula IVh.

In one embodiment, the composition is characterized in that the hydrophobic radical is a radical according to formula I, Ia, Ic or Id wherein r=1, GpR is a radical according to formula II, R is a radical according to formula Z, I=1, GpI is a radical according to formula IIIa and GpC is a radical according to formulaIVh.

In one embodiment, the composition is characterized in that the hydrophobic radical is a radical according to formula I, Ia, Ib or Id wherein r=2, GpR₁ and GpR₂, which are different,are radicals according to formula II and II [symbol] respectively, R₁ is a radical according to formula Z and R₂ is a radical according to formula Z [symbol] or Z″, i=1, GpI is a radical according to formula IIIa and GpC is a radical according to formula IVh.

In one embodiment, the composition is characterized in that the hydrophobic radical is a radical according to formula I, Ia, or Ib, wherein r=2, GpR₁ and GpR₂, different, are radicals according to formula II and II [symbol] respectively, R₁ is a radical according to formula Z and R₂ is a radical according to formula Z [symbol] or Z″, i=2, GpI₁ and GpI₂, identical, are radicals according to Formula IIIa, and GpC is a radical according to formula IVh.

In one embodiment, the composition is characterized in that the hydrophobic radical is a radical according to formula I, Ia, Ib or Id wherein r=2 or 3, GpR₁ is a radical according to formula II and R₁ is an alkyl radical comprising from 2 to 12 carbon atoms, GpR₂ and GpR₃, which are identical or different, are radicals according to formula II [symbol] , R₂ and R₃, which are identical or different, are radicals according to formulas Z [symbol] and Z″, i=1, 2 or 3, GpI₁, GpI₂, and GpI₃ which are identical are radicals according to Formula IIIa, and GpC is a radical according to formula IVh.

In one embodiment, the composition is characterized in that the hydrophobic radical is a radical according to formula I, Ia, Ib or Id wherein r=2 or 3, GpR₁ is radical according to formula II and R₁ is an alkyl radical comprising from 2 to 12 carbon atoms, GpR₂ and optionally GpR₃ are radicals according to formula II [symbol] radicals, R is a radical selected according to formulas Z [symbol] and Z″, i=1 or 2, identical GpI₁ and GpI₂ are radicals according to formula IIIa, and GpC is a radical according to formulaIVh.

In one embodiment, the composition is characterized in that the hydrophobic radical is a radical according to formula I, Ia, Ib or Id wherein r=2, GpR₁ is radical according to formula II and R₁ is an alkyl radical comprising from 2 to 12 carbon atoms, GpR₂ is a radical according to formula II [symbol] , R is a radical according to formulas Z [symbol] and Z″, i=1, GpI is a radical according to formula IIIa and GpC is a radical according to formula IVh.

In one embodiment, the composition is characterized in that the hydrophobic radical is a radical according to formula I, Ia, Ib or Id wherein r=2, GpR₁ is radical according to formula II and R₁ is an alkyl radical comprising 2 carbon atoms, GpR₂ is a radical according to formula II [symbol] , R is a radical according to formulas Z [symbol] and Z″, i=2, GpI1 and GpI2 which are identical are radicals according to formula Ma, and GpC is a radical according to formula IVh.

In one embodiment, the composition is characterized in that the precursor of the hydrophilic backbone HB bearing at least one hydrophobic radical is a polymer the repeating units of which are chosen in the group consisting of the lysine group, glutamic acid aspartic acid, and the ethers, in particular ethylene glycol and propylene glycol.

According to one particular embodiment, the polyethers have two extremities. According to one particular embodiment, the extremitiesof the polyethers are two amines, two acids or one amine and one acid.

In one embodiment, the composition according to the invention is characterized in that the hydrophilic backbone HB is a co-polyamino acid chosen among the polyglutamates hereinafter referred to as PLG.

In one embodiment, the composition according to the invention is characterized in that the hydrophilic backbone HB is a copolyamino acid PLG bearing hydrophobic radicals, said hydrophilic backbone is chosen among the copolyaminoacids according to the following formula XXX:

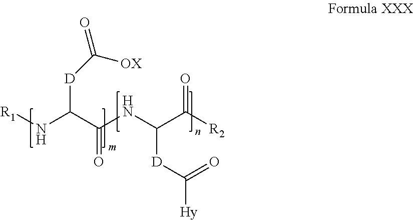

wherein,
D is, independently, either a —CH₂— group (aspartic unit) or a —CH₂—CH₂— group (glutamic unit),
R₁ is a hydrophobic radical selected from the hydrophobic radicals -Hy, or a radical chosen in the group consisting of a H, a linear acyl group in C₂ to C₁₀, a branched acyl group in C₃ to C₁₀, a benzyl, a terminal “amino acid” unit and a pyroglutamate,
R₂ is either a hydrophobic radical selected from the hydrophobic radical -Hy, or a radical chosen in the group consisting of an —OH, an amine group, a terminal “amino acid” unit and a pyroglutamate,
said copolyamino acid comprises at least one hydrophobic radical -Hy as defined above,
X represents a cationic entity selected from the group comprising alkaline cations;
if n=0 then m≥1
if m=0 then n≥1
n+m represents the degree of polymerization DP of the copolyamino acid, i.e., the average number of monomer units per chain of copolyamino acid and 5≤n+m≤250 and
the ratio M between the number of hydrophobic radicals and the number of repetition units being comprised from 0<M≤0.5.

In one embodiment, the composition is characterized in that the copolyamino acid bearing hydrophobic radicals is chosen among the following copolyamino acids according to Formula XXX wherein n=0 according to the following formula XXXe:

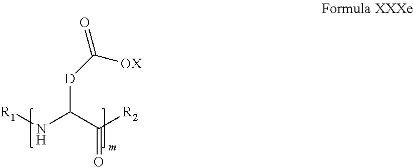

wherein m, X, D, R₁ and R₂ have the definitions given above and at least R₁ or R₂ is a hydrophobic radical Hy.

In one embodiment, the composition according to the invention is characterized in that the copolyamino acid bearing at least one hydrophobic radical -Hy is chosen among the copolyamino acids according to formula XXXe wherein R₁ is a hydrophobic radical -Hy and R₂ is not a hydrophobic radical -Hy.

In one embodiment, the composition according to the invention is characterized in that the copolyamino acid bearing at least one hydrophobic radical -Hy is chosen among the copolyamino acids according to formula XXXe wherein R₂ is a hydrophobic radical -Hy and R₁ is not a hydrophobic radical -Hy.

In one embodiment, the composition according to the invention is characterized in that the copolyamino acid bearing at least one hydrophobic radical -Hy is chosen among the copolyamino acids according to formula XXXe wherein R₁ and R₂ are identical or different hydrophobic radicals -Hy.

In one embodiment, the composition is characterized in that the copolyamino acid bearing hydrophobic radicals is chosen among the copolyamino acids according to the following Formula XXX wherein m=0 according to the following formula XXXf:

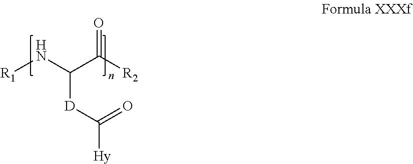

wherein n, X, D, R₁ and R₂ have the definitions given above.

In one embodiment, the composition according to the invention is characterized in that the copolyamino acid bearing at least one hydrophobic radical -Hy is chosen among the copolyamino acids according to formula XXXf wherein R₁ is a hydrophobic radical -Hy and R₂ is not a hydrophobic radical -Hy.

In one embodiment, the composition according to the invention is characterized in that the copolyamino acid bearing at least one hydrophobic radical -Hy is chosen among the copolyamino acids according to formula XXXf wherein R₂ is a hydrophobic radical -Hy and R₁ is not a hydrophobic radical -Hy.

In one embodiment, the composition according to the invention is characterized in that the copolyamino acid bearing at least one hydrophobic radical -Hy is chosen among the copolyamino acids according to formula XXXf wherein R₁ and R₂ are not hydrophobic radicals -Hy.

In one embodiment, the composition according to the invention is characterized in that the copolyamino acid bearing at least one hydrophobic radical -Hy is chosen among the copolyamino acids according to formula XXXf wherein R₁ and R₂ are identical or different hydrophobic radicals -Hy.

In one embodiment, the composition according to the invention is characterized in that the copolyamino acid bearing at least one hydrophobic radical -Hy is chosen among the copolyamino acids according to the following formula XXXa:

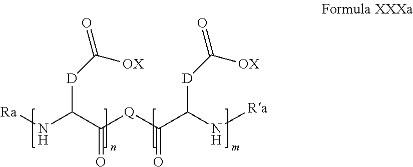

wherein,
D and X have the definitions given above,
Ra and R [symbol] , whether they are identical or different, are either a hydrophobic radical -Hy, or a radical chosen in the group consisting of an H, a linear acyl group in C₂ to C₁₀, a branched acyl group in C₃ to C₁₀, a benzyl, a terminal “amino acid” unit and a pyroglutamate,
at least one of Ra and R′a is a hydrophobic radical [symbol] Hy,
Hy has the meaning given above.
Q is a spacer binding at least two chains of glutamic or aspartic PLG units according to formula Q[ [symbol] *]ₖ, linear or branched, at least divalent constituted by an alkyl chain comprising one or more heteroatoms chosen in the group consisting of nitrogen and oxygen atoms and/or bearing one or more heteroatoms constituted by nitrogen and oxygen atoms and/or radicals bearing one or more heteroatoms constituted by nitrogen and oxygen atoms and/or carboxyl functions and optionally bearing at least one hydrophobic radical -Hy.
n+m have the same definitions given above.

In one embodiment, the Q[—*]ₖ radical or spacer is represented by a radical according to formula QII:

Q[—*]ₖ=([Q′]_{q})[—*]ₖ Formula QII
wherein 1≤q≤5,
k≥2
The radicals Q′ are identical or different and chosen in the group consisting of the radicals according to the following formulas QIII to QVI [symbol] , to form Q[ [symbol] *]ₖ: by a radical according to formula QIII:

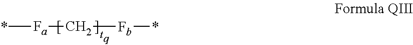

wherein 1≤t_{q}≤8,
by a radical according to formula QIV:

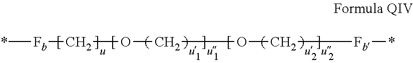

wherein:
At least one of the u₁″ or u₂″ is different from 0.
If u″₁≠0 then u′₁≠0 and if u″₂≠0 then u′₂≠0,
u [symbol] and u [symbol] are identical or different and,
2≤u≤4,
0≤u [symbol] ≤4,
0≤u″₁≤4,
0≤u [symbol] ≤4,
0≤u″₂≤4;
   by a radical according to formula QV:

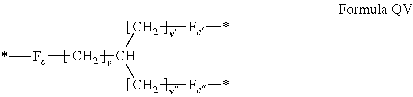

wherein:
v, v′ and v″ whether they are identical or different, are integers ≥0, and v+v′+v″≤15, by a radical according to formula QVI:

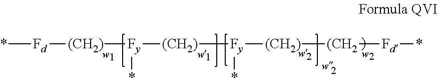

wherein:
w′₁ is different from 0,
0≤w″₂≤1,
w₁≤6 and w [symbol] ≤6 and/or w₂≤6 and w [symbol] ≤6
   with identical or different Fₓ=Fₐ, F_{b}, F_{c}, F_{d}, Fₐ [symbol] F_{b} [symbol] F_{c} [symbol] F_{c} [symbol] and F_{d} [symbol] representing —NH— or —CO— functions and F_{y} representing a trivalent nitrogen atom —N═,
   two radicals Q [symbol] being bound together by a covalent bond between a carbonyl function, Fₓ=—CO—, and an amine function Fₓ=—NH— or F_{y}=—N═, thus forming an amide bond.

In one embodiment k is 2, 3, 4, 5 or 6.

In one embodiment k=2.

In one embodiment q=1.

In one embodiment k is 2 and q=1.

In one embodiment, said radical Q [symbol] is chosen among the radicals according to formula QVI, wherein w₂=0 according to formula QVI [symbol] s defined below:

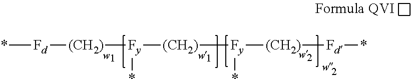

wherein:
w′₁ is different from 0,
0≤w″₂≤1,
w₁≤6 and w [symbol] ≤6 and/or w₂≤6
with F_{d}, and F_{d}′ being identical or different, representing —NH— or —CO— functions and F_{y} representing a trivalent nitrogen atom —N═,
two radicals Q′ being bound together by a covalent bond between a carbonyl function, Fₓ=—CO—, and an amine function Fₓ=—NH— or F_{y}=—N═, thus forming an amide bond, where in each of the radicals shown above, Fₓ=Fₐ, F_{b}, F_{c}, F_{d}, Fₐ [symbol] F_{b} [symbol] F_{c} [symbol] H and F_{d} [symbol] which are identical or different, representing —NH— or —CO— functions and F_{y} representing a trivalent nitrogen atom —N═, two radicals Q′ being bound together by a covalent bond between a carbonyl function, Fₓ=—CO—, and an amine function Fₓ=—NH— or F_{y}=—N═, thus forming an amide bond. When a function Fₓ=Fₐ, F_{b}, F_{c}, F_{d}, Fₐ [symbol] F_{b} [symbol] F_{c} [symbol] F_{c} [symbol] and F_{d} [symbol] is not used in a bond between two Q [symbol] , this function is then free and salified

In one embodiment, if Fₐ and Fₐ [symbol] are —NH—, then t≥2.

In one embodiment, if Fₐ and Fₐ [symbol] are —CO—, then t≥1.

In one embodiment, if Fₐ and Fₐ [symbol] are —CO— and —NH—, then t≤1.

In one embodiment, if F_{b} and F_{b} [symbol] are —NH—, then u and u [symbol] ≥2 and/or u [symbol] ≥2.

In one embodiment, if F_{c}, F_{c′} and F_{c″} are —NH— then at least two of v, v′ and v″ are different than 0.

In one embodiment, if F_{c}, F_{c} [symbol] and F_{c″} are 2 —NH— and 1 —CO— then at least one of the indices of —(CH₂)— bearing a nitrogen is different from 0.

In one embodiment, if F_{c}, F_{c} [symbol] and F_{c″} are 1 —NH— and 2 —CO— then no conditions.

In one embodiment, if F_{c}, F_{c′} and F_{c″} are —CO— then at least one of v, v′ and v″ is different than 0.

In one embodiment, if F_{d} and F_{d} [symbol] are —NH—, w₁ and w [symbol] ≥2 and/or w₂ and w [symbol] ≥2.

In one embodiment, if F_{d} and F_{d} [symbol] are —CO—, w₁ and w [symbol] ≥1 and/or w₂ and w [symbol] ≥1.

In one embodiment, if F_{d} and F_{d} [symbol] are —CO—, and —NH—, w₁ and w [symbol] ≥1 and/or w₂ and w [symbol] ≥1.

The at least two chains of glutamic or aspartic PLG units being bound to Q[ [symbol] *]ₖ by a Fₓ or F_{y} function by a covalent bond to form an amide bond with an —NH— or —CO— function of the PLG.

In one embodiment, 1≤q≤5.

In one embodiment, v+v′+v″≥15.

In one embodiment, at least one of the Q [symbol] is a radical according to formula III,

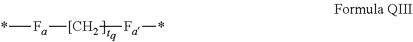

wherein the precursor is a diamine.

In one embodiment, the precursor of the radical according to formula QIII is a diamine chosen in the group consisting of ethylenediamine, butylenediamine, l [symbol] exylénediamine, 1,3-diaminopropane and 1,5-diaminopentane.

In one embodiment, t_{q}=2 and the precursor of the radical according to formula QIII is ethylenediamine.

In one embodiment, t_{q}=4 and the precursor of the radical according to formula QIII is butylenediamine.

In one embodiment, t_{q}=6 and the precursor of the radical according to formula QIII is hexylenediamine.

In one embodiment, t_{q}=3 and the precursor of the radical according to formula QIII is 1,3-diaminopropane.

In one embodiment, t_{q}=5 and the precursor of the radical according to formula QIII is 1,5-diaminopentane.

In one embodiment, the precursor of the radical according to formula QIII is an amino acid.

In one embodiment, the precursor of the radical according to formula QIII is an amino acid chosen in the group consisting of aminobutanoic acid, aminohexanoic acid and beta-alanine.

In one embodiment, t_{q}=2 and the precursor of the radical according to formula QIII is beta-alanine.

In one embodiment, t_{q}=6 and the precursor of the radical according to formula III is aminohexanoic acid.

In one embodiment, t_{q}=4 and the precursor of the radical according to formula QIII is aminobutanoic acid.

In one embodiment, the precursor of the radical according to formula QIII is a diacid.

In one embodiment, the precursor of the radical according to formula III is a diacid. chosen in the group consisting of succinic acid, glutaric acid and adipic acid.

In one embodiment, t_{q}=2 and the precursor of the radical according to formula QIII is succinic acid.

In one embodiment, t_{q}=3 and the precursor of the radical according to formula QIII is glutaric acid.

In one embodiment, t_{q}=4 and the precursor of the radical according to formula QIII is adipic acid.

In one embodiment, at least one of the Q [symbol] is a radical according to formula QIV,

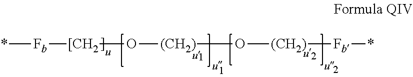

wherein the precursor is a diamine.

In one embodiment, the precursor of the radical according to formula QIV is a diamine chosen in the group consisting of diethyleneglycol diamine, triethyleneglycol diamine, 4,9-dioxa-1,12-dodecanediamine and 1-amino-4,7,10-trioxa-13-tridecanamine.

In one embodiment, u=u [symbol] =2, u″₁=1, u″₂=0 and the precursor of the radical according to formula QIV is diethyleneglycol diamine.

In one embodiment, u=u [symbol] =u [symbol] , =2, u″₁=u″₂=1 and the precursor of the radical according to formula QIV is diethylene glycol diamine.

In one embodiment, u=u [symbol] =3, u [symbol] =4, u″₁=u″₂=1 and the precursor of the radical according to formula QIV is 4,9-dioxa-1,12-dodecanediamine.

In one embodiment, u=u [symbol] 3, u [symbol] =u″₁=2, u″₂=1 and the precursor of the radical according to formula QIV is 4,7,10-trioxa-1,13-tridecanediamine.

In one embodiment, at least one of the Q [symbol] is a radical according to formula QV,

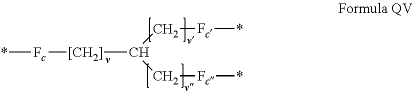

wherein the precursor is chosen in the group consisting of amino acids.

In one embodiment, the precursor of the radical according to formula QV is an amino acid chosen in the group consisting of lysine, ornithine, and 2,3-diaminopropionic acid.

In one embodiment, v=4, v [symbol] =v″=0 and the precursor of the radical according to formula V radical is lysine.

In one embodiment, v=3, v [symbol] =v″=0 and the precursor of the radical according to formula V radical is ornithine.

In one embodiment, v=2, v [symbol] =v″=0 and the precursor of the radical according to formula V radical is 2,3-diaminopropionic acid.

In one embodiment, at least one of the Q [symbol] is a radical according to formula QV,

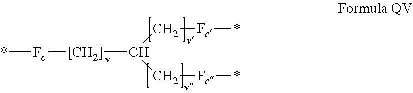

wherein the precursor is chosen in the group consisting of triacids.

In one embodiment, the precursor of the radical according to formula QV is a triacid chosen in the group consisting of tricarballylic acid.

In one embodiment, v=0, v [symbol] =″=1 and the precursor of the radical according to formula QV is tricarballylic acid.

In one embodiment, at least one of the Q [symbol] is a radical according to formula QV,

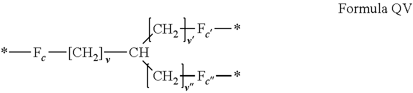

wherein the precursor is chosen in the group consisting of triamines.

In one embodiment, the precursor of the radical according to formula QV is a triamine chosen in the group consisting of (2-(aminomethyl)propane-1,3-diamine).

In one embodiment, v=v [symbol] =v″=1 and the precursor of the radical according to formula QV is (2-(aminomethyl) propane-1,3-diamine).

In one embodiment, at least one of the Q [symbol] is a radical according to Formula QVI,

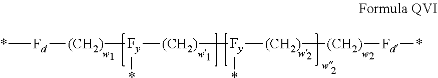

wherein the precursor is a triamine.

In one embodiment, w″₂=0 and the precursor of the radical according to formula QVI is a triamine chosen in the group consisting of spermidine, norspermidine, and diethylene triamine and bis(hexamethylene) triamine.

In one embodiment, w″₂=0 and the precursor of the radical according to formula QVI is spermidine.

In one embodiment, w″₂=0 and the precursor of the radical according to formula QVI is norspermidine.

In one embodiment, w″₂=0 and the precursor of the radical according to formula QVI is diethylene triamine.

an embodiment, w″₂=0 and the precursor of the radical according to formula QVI is bis(hexamethylene)triamine.

In one embodiment, at least one of the Q [symbol] is a radical according to formula QVI,

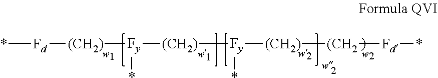

wherein the precursor is a tetramine.

In one embodiment, w″₂=1 and the precursor of the radical according to formula QVI is a tetramine.

In one embodiment, w″₂=1 and the precursor of the radical according to formula QVI is a tetramine chosen in the group consisting of spermine and triethylene tetramine acid.

In one embodiment, w″₂=1 and the precursor of the radical according to formula QVI is spermine.

In one embodiment, w″₂=1 and the precursor of the radical according to formula QVI is triethylene tetramine.

In one embodiment, the precursor of the radical or spacer Q[ [symbol] *]ₖ has 4 reactive functions, chosen among the group of amine functions and carboxylic acid functions.

In one embodiment, the precursor of the radical or spacer Q[ [symbol] *]ₖ has 4 reactive functions and the precursor of the radical or spacer Q[ [symbol] *]ₖ is 1,2,3,4-butanetetraoic acid.

In one embodiment, at least one of the Q [symbol] is a radical according to formula QVI [symbol] ,

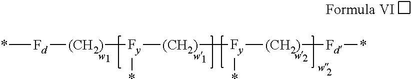

wherein the precursor is a triamine.

In one embodiment, w″₂=0 and the precursor of the radical according to formula QVI′ is a triamine, chosen in the group consisting of spermidine, norspermidine, and diethylene triamine and bis(hexamethylene)triamine.

In one embodiment, w″₂=0 and the precursor of the radical according to formula QVI′ is spermidine.

In one embodiment, w″₂=0 and the precursor of the radical according to formula QVI′ is norspermidine.

In one embodiment, w″₂=0 and the precursor of the radical according to formula QVI′ is diethylene triamine.

In one embodiment, w″₂=0 and the precursor of the radical according to formula QVI′ is bis(hexamethylene)triamine.

In one embodiment, at least one of the Q [symbol] is a radical according to formula QVI [symbol] radical,

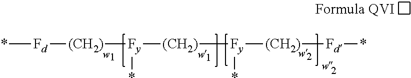

wherein the precursor is a tetramine.

In one embodiment, w″₂=1 and the precursor of the radical according to formula QVI′ is a tetramine.

In one embodiment, w″₂=1 and the precursor of the radical according to formula QVI′ is a tetramine chosen in the group consisting of spermine and triethylene tetramine.

In one embodiment, w″₂=1 and the precursor of the radical according to formula QVI′ is spermine.

In one embodiment, w″₂=1 and the precursor of the radical according to formula QVI′ is triethylene tetramine.

In one embodiment, all Fₓ are bound to the PLG or to other Fₓ or F_{y}.

In one embodiment, one or more of the Fx are free, i.e., not bound to the PLG, or to another Fₓ, or to an F_{y}.

In one embodiment, one Fₓ is free, i.e., not bound to the PLG, or to another Fₓ, or to an F_{y}.

In one embodiment, the Fₓ(es) of the —CO— type is free, it is in the form of a carboxylic acid salt.

In one embodiment, the Fₓ of the free —CO— type is borne by a radical Q [symbol] a according to formula QV.

In one embodiment, the the —NH— type Fₓ(s) is free, it is in the amine or ammonium form.

In one embodiment, the PLGs are bound to Fₓ with Fₓ=—NH— or to F_{y} by at least one carbonyl function of the PLG.

In one embodiment, the PLGs are bound to Fₓ with Fₓ=—NH— or to F_{y} by at least one carbonyl function that is not in the C-terminal position of the PLG.

In one embodiment, the PLGs are bound to Fₓ with Fₓ=—NH— or to F_{y} by the carbonyl function in the C-terminal position of the PLG.

In one embodiment, the PLGs are bound to Fₓ with Fₓ=—NH— by the carbonyl function in the C-terminal position of the PLG.

In one embodiment, the PLGs are bound to Fₓ with Fₓ=F_{y} by the carbonyl function in the C-terminal position of the PLG.

In one embodiment, the PLGs are bound to Fₓ with Fₓ=—CO— by the nitrogen atom in the N-terminal position of the PLG.

In one embodiment, the composition according to the invention is characterized in that the copolyamino acid bearing at least one hydrophobic radical [symbol] Hy is chosen among the copolyamino acids according to formula XXXa wherein Rₐ and R [symbol] which are identical, are a hydrophobic radical [symbol] Hy.

In one embodiment, the composition according to the invention is characterized in that the copolyamino acid bearing at least one hydrophobic radical [symbol] Hy is chosen among the copolyamino acids according to formula XXXa wherein Rₐ and R [symbol] which are different, are a hydrophobic radical [symbol] Hy.

In one embodiment, the composition according to the invention is characterized in that the copolyamino acid bearing at least one hydrophobic radical [symbol] Hy is chosen among the copolyamino acids according to formula XXXa wherein Rₐ is a hydrophobic radical [symbol] Hy and R [symbol] is not a hydrophobic radical [symbol] Hy.

In one embodiment, the composition according to the invention is characterized in that the copolyamino acid bearing at least one hydrophobic radical [symbol] Hy is chosen among the copolyamino acids according to formula XXXa wherein R [symbol] is a hydrophobic radical [symbol] Hy and Rₐ is not a hydrophobic radical [symbol] Hy.

In one embodiment, the composition according to the invention is characterized in that the copolyamino acid bearing at least one hydrophobic radical [symbol] Hy is chosen among the copolyamino acids according to the following formula XXXa

[symbol]

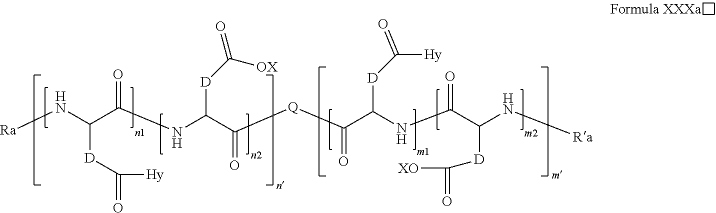

### Wherein:

D, X, Ra and R [symbol] have the definitions given above,
Q and Hy have the meanings given above,
n₁+m₁ represents the number of glutamic units or aspartic units of the copolyamino acid chains bearing an -Hy radical,
n₂+m₂ represents the number of glutamic units or aspartic units of the copolyamino acid chains not bearing an [symbol] Hy radical,
n₁+n₂=n′ and m₁+m₂=m [symbol]
n′+m′ represents the degree of polymerization DP of the copolyamino acid, that is to say, the average number of monomeric units per copolyamino acid chain and 5≤n′+m′≤250.

In one embodiment, the composition according to the invention is characterized in that the copolyamino acid bearing at least one hydrophobic radical [symbol] Hy is chosen among the copolyamino acids according to formula XXXa [symbol] wherein Rₐ and R [symbol] and which are identical, are a hydrophobic radical [symbol] Hy.

In one embodiment, the composition according to the invention is characterized in that the copolyamino acid bearing at least one hydrophobic radical [symbol] Hy is chosen among the copolyamino acids according to formula XXXa [symbol] wherein Ra and R [symbol] , which are different, are a hydrophobic radical [symbol] Hy.

In one embodiment, the composition according to the invention is characterized in that the copolyamino acid bearing at least one hydrophobic radical [symbol] Hy is chosen among the copolyamino acids according to formula XXXa [symbol] , wherein Ra is a hydrophobic radical [symbol] Hy and R [symbol] is not a hydrophobic radical [symbol] Hy.

In one embodiment, the composition according to the invention is characterized in that the copolyamino acid bearing at least one hydrophobic radical [symbol] Hy is chosen among the copolyamino acids according to formula XXXa [symbol] wherein R [symbol] is a hydrophobic radical [symbol] Hy and R [symbol] is not a hydrophobic radical [symbol] Hy.

In one embodiment, the composition according to the invention is characterized in that the copolyamino acid bearing at least one hydrophobic radical [symbol] Hy is chosen among the copolyamino acids according to the following formula XXXb:

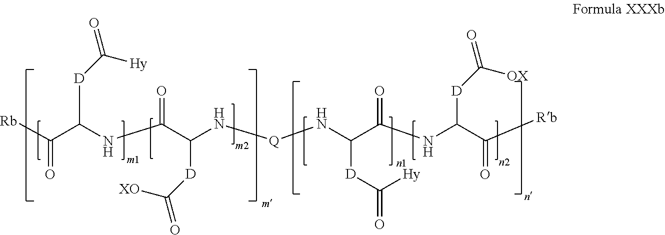

wherein,
D and X have the definitions given above,
Rb and Rb [symbol] , which may be identical or different, are either a hydrophobic radical -Hy or a radical chosen in the group consisting of —OH, an amine group, a terminal “amino acid” unit and a pyroglutamate,
at least one of Rb and R [symbol] is a hydrophobic radical -Hy,
Q and Hy have the meanings given above.
n+m have the same definitions given above.

In one embodiment, the composition according to the invention is characterized in that the copolyamino acid bearing at least one hydrophobic radical [symbol] Hy is chosen among the copolyamino acids according to formula XXXb wherein Rb and R [symbol] , which are identical, are a hydrophobic radical -Hy.

In one embodiment, the composition according to the invention is characterized in that the copolyamino acid bearing at least one hydrophobic radical [symbol] Hy is chosen among the copolyamino acids according to formula XXXb wherein Ra and R [symbol] , which are different, are a hydrophobic radical [symbol] Hy.

In one embodiment, the composition according to the invention is characterized in that the copolyamino acid bearing at least one hydrophobic radical [symbol] Hy is chosen among the copolyamino acids according to formula XXXb wherein Rb is a hydrophobic radical [symbol] Hy and R [symbol] is not a hydrophobic radical [symbol] Hy.

In one embodiment, the composition according to the invention is characterized in that the copolyamino acid bearing at least one hydrophobic radical [symbol] Hy is chosen among the copolyamino acids according to formula XXXb wherein R [symbol] is a hydrophobic radical [symbol] Hy and Rb is not a hydrophobic radical [symbol] Hy.

In one embodiment, the composition according to the invention is characterized in that the copolyamino acid bearing at least one hydrophobic radical [symbol] Hy is chosen among the copolyamino acids according to the following formula XXXb

[symbol]

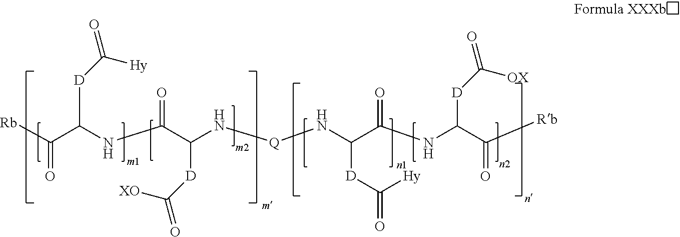

wherein: -p1 D and X have the definitions given above,
Q and Hy have the meanings given above.
Rb and Rb [symbol] , which may be identical or different, are either a hydrophobic radical -Hy or a radical chosen in the group consisting of —OH, an amine group, a terminal “amino acid” unit and a pyroglutamate,
at least one of Rb and R [symbol] is a hydrophobic radical -Hy,
n₁+m₁ represents the number of glutamic units or aspartic units of the copolyamino acid chains bearing an -Hy radical,
n₂+m₂ represents the number of glutamic units or aspartic units of the copolyamino acid chains not bearing an -Hy radical,
n₁+n₂=n′ and m1+m2=m [symbol]
n′+m′ represents the degree of polymerization DP of the copolyamino acid, that is to say, the average number of monomeric units per copolyamino acid chain and 5≤n′+m′≤250.

In one embodiment, the composition according to the invention is characterized in that the copolyamino acid bearing at least one hydrophobic radical [symbol] Hy is chosen among the copolyamino acids according to formula XXXb [symbol] wherein Rb and R [symbol] , which are identical, are a hydrophobic radical -Hy.

In one embodiment, the composition according to the invention is characterized in that the copolyamino acid bearing at least one hydrophobic radical [symbol] Hy is chosen among the copolyamino acids according to formula XXXb [symbol] wherein Rb and R [symbol] , which are different, are a hydrophobic radical [symbol] Hy.

In one embodiment, the composition according to the invention is characterized in that the copolyamino acid bearing at least one hydrophobic radical [symbol] Hy is chosen among the copolyamino acids according to formula XXXb [symbol] wherein Rb is a hydrophobic radical [symbol] Hy and R [symbol] is not a hydrophobic radical [symbol] Hy.

In one embodiment, the composition according to the invention is characterized in that the copolyamino acid bearing at least one hydrophobic radical [symbol] Hy is chosen among the copolyamino acids according to formula XXXb [symbol] wherein R [symbol] is a hydrophobic radical [symbol] Hy and R [symbol] is not a hydrophobic radical [symbol] Hy.

In one embodiment, the composition according to the invention is characterized in that when the copolyamino acids comprise aspartate units, then the copolyamino acids may also comprise monomeric units according to formula VIII and/or VIII

[symbol]

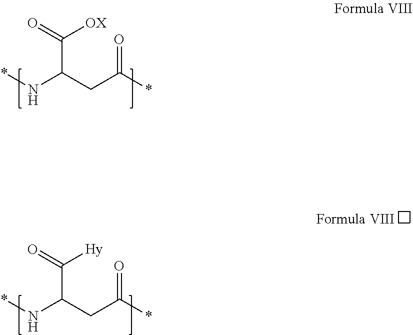

In one embodiment, the composition is characterized in that the copolyamino acid bearing hydrophobic radicals is chosen among the copolyamino acids according to formulas XXX, XXXe, XXXf, XXXa, XXXb, XXXa [symbol] or XXXb [symbol] wherein group D is a —CH₂—CH₂— group (glutamic unit).

In one embodiment, the composition is characterized in that the copolyamino acid bearing carboxylate charges and hydrophobic radicals is chosen among the copolyamino acids according to formulas XXX, XXXa, XXXb, XXXe, XXXf, XXXa [symbol] or XXXb [symbol] wherein group D is a —CH₂— group (aspartic unit).

In one embodiment, the composition according to the invention is characterized in that the hydrophilic backbone HB is a polylysine bearing hydrophobic radicals and said hydrophilic backbone is chosen among the polylysines according to the following formula XXXX:

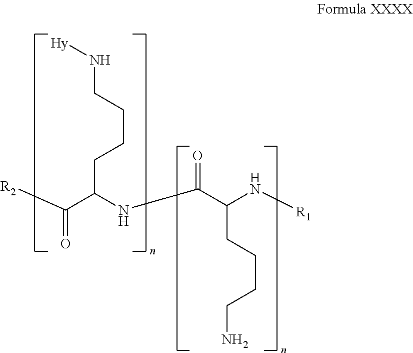

wherein,
R₁ is a hydrophobic radical selected from the hydrophobic radicals [symbol] Hy, or a radical chosen in the group consisting of an [symbol] H or a terminal [symbol] amino acid [symbol] unit,
R₂ is either a hydrophobic radical chosen among the hydrophobic radicals [symbol] Hy, or a radical chosen in the group consisting of an [symbol] OH, an amine group or a terminal [symbol] amino acid [symbol] unit,
said polylysine comprises at least one hydrophobic radical [symbol] Hy as defined above,
if n=0 then m≥1
if m=0 then n≥1
n+m represents the degree of PD polymerization of the polylysine, that is to say, the average number of monomeric units per copolyamino acid chain and 5≤n+m≤250.
the ratio M between the number of hydrophobic radicals and the number of glutamic or aspartic units being comprised from 0<M≤0.5

In one embodiment, the composition according to the invention is characterized in that the hydrophilic backbone HB is a polylysine bearing at least one hydrophobic radical and said hydrophilic backbone is chosen among the polylysines according to the following formula XXXXa:

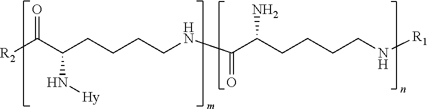

Wherein, R₁, R₂, Hy, m and n have the meanings given above.

According to one particular embodiment, m=0 and R₁ and/or R₂ is a hydrophobic radical [symbol] Hy.

In one embodiment, the composition according to the invention is characterized in that n+m is from 10 to 250.

In one embodiment, the composition according to the invention is characterized in that n+m is comprised from 10 to 200.

In one embodiment, the composition according to the invention is characterized in that n+m is comprised from 15 to 150.

In one embodiment, the composition according to the invention is characterized in that n+m is comprised from 15 to 100.

In one embodiment, the composition according to the invention is characterized in that n+m is comprised from 15 to 80.

In one embodiment, the composition according to the invention is characterized in that n+m is comprised from 15 to 65.

In one embodiment, the composition according to the invention is characterized in that n+m is comprised from 20 to 60.

In one embodiment, the composition according to the invention is characterized in that n+m is comprised from 20 to 50.

In one embodiment, the composition according to the invention is characterized in that n+m is comprised from 20 to 40.

In one embodiment, the composition according to the invention is characterized in that the hydrophilic backbone HB is a polyalkylene glycol bearing hydrophobic radicals and said hydrophilic backbone is chosen among the polyalkylene glycols according to the following formula XXXXXa

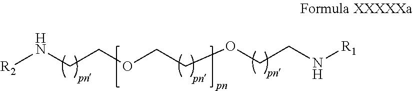

wherein,
R₁ is a hydrophobic radical chosen among the hydrophobic radicals Hy, or a radical chosen in the group consisting of an [symbol] H or [symbol] OH,
R₂ is either a hydrophobic radical chosen among the hydrophobic radicals Hy, or a radical chosen in the group consisting of an [symbol] OH or [symbol] H,
and at least one among R₁ or R₂ is a hydrophobic radical [symbol] Hy.
pn [symbol] is an integer from 1 to 5, 1≤pn [symbol] ≤5
pn represents the degree of polymerization DP of the polyalkylene glycol, i.e., the average number of monomer units per polyalkylene glycol chain and 5≤n+m≤250.

In one embodiment, the composition according to the invention is characterized in that the hydrophilic backbone HB is a polyalkylene glycol bearing hydrophobic radicals and said hydrophilic backbone is chosen among the polyalkylene glycols according to the following formula XXXXXb:

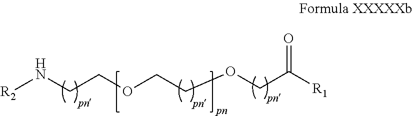

R₁ is a hydrophobic radical chosen among the hydrophobic radicals Hy, or an —OH radical,
R₂ is a hydrophobic radical chosen among the hydrophobic radicals [symbol] Hy, or an [symbol] H radical,
and at least one among R₁ or R₂ is a hydrophobic radical [symbol] Hy.
pn [symbol] is an integer from 1 to 5, 1≤pn [symbol] ≤5
pn represents the degree of polymerization DP of the polyalkylene glycol, i.e., the average number of monomer units per polyalkylene glycol chain and 5≤n+m≤250.

In one embodiment, the composition according to the invention is characterized in that the hydrophilic backbone HB is a polyalkylene glycol bearing hydrophobic radicals and said hydrophilic backbone is chosen among among the polyalkylene glycols according to the following formula XXXXXc

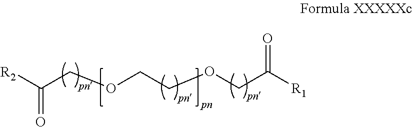

R₁ is a hydrophobic radical chosen among the hydrophobic radicals Hy, or an [symbol] OH radical,
R₂ is a hydrophobic radical chosen among the hydrophobic radicals [symbol] Hy, or an [symbol] OH radical,
pn [symbol] is an integer from 1 to 5, 1≤pn [symbol] ≤5
pn represents the degree of PD polymerization of the polyalkylene glycol, i.e., the average number of monomer units per polyalkylene glycol chain and 5≤n+m≤250.

In one embodiment, the precursors of the polyalkylene glycols according to formula XXXXXa, XXXXXb or XXXXXc are chosen in the group consisting of the polyalkylene glycols according to formulas XXXXX [symbol] , XXXXX [symbol] or XXXXX [symbol] shown below:

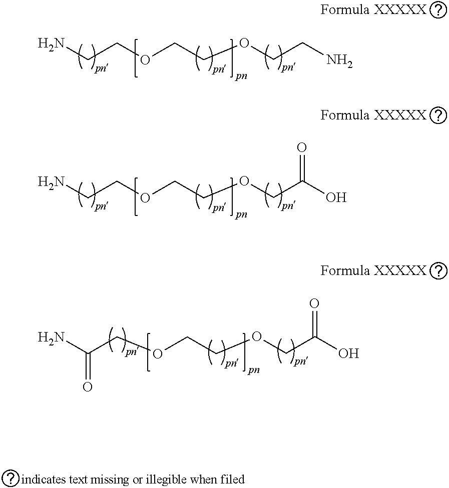

### Wherein:

pn [symbol] is an integer from 1 to 5, 1≤pn [symbol] 5
pn represents the degree of polymerization DP of the polyalkylene glycol, i.e., the average number of monomer units per polyalkylene glycol chain and 5≤n+m≤250.

In one embodiment, the composition according to the invention is characterized in that pn is comprised from 10 to 250.

In one embodiment, the composition according to the invention is characterized in that pn is comprised from 10 to 200.

In one embodiment, the composition according to the invention is characterized in that pn is comprised from 15 to 150.

In one embodiment, the composition according to the invention is characterized in that pn is comprised from 15 to 100.

In one embodiment, the composition according to the invention is characterized in that pn is comprised from 15 to 80.

In one embodiment, the composition according to the invention is characterized in that pn is comprised from 15 to 65.

In one embodiment, the composition according to the invention is characterized in that pn is comprised from 20 to 60.

In one embodiment, the composition according to the invention is characterized in that pn is comprised from 20 to 50.

In one embodiment, the composition according to the invention is characterized in that pn is comprised from 20 to 40.

The invention also relates to said amphiphilic compounds comprising a hydrophilic HB backbone bearing hydrophobic radicals according to Formula I and the precursors of said hydrophobic radicals.

In one embodiment, the invention also relates to said amphiphilic compounds comprising a hydrophilic backbone HB, substituted by at least one hydrophobic radical -Hy according to formula I:

*-(GpR)ᵣ₋(GpI)ᵢ₋[(GpR)ᵣ [symbol] (GpI)ᵢ [symbol] ]ₜ₋GpC Formula I

wherein,
GpI is a divalent radical, said radical comprising at least one imidazole unit Im according to formula III:

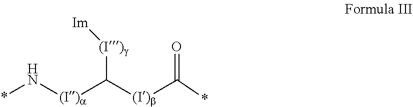

GpR is a radical according to formulas II, II [symbol] or II″:

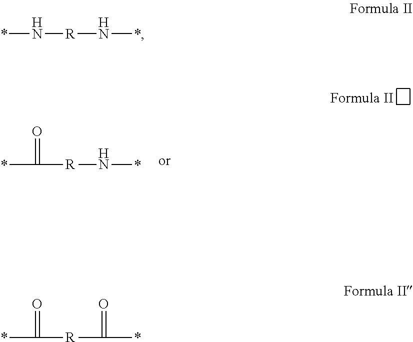

GpC is a radical according to Formula IV:

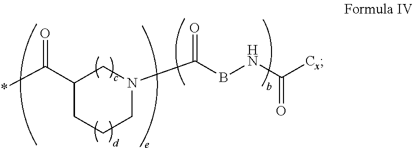

the * indicate the attachment sites of the hydrophobic radical [symbol] Hy to the hydrophilic backbone HB or the above radicals (I, II, II [symbol] , II″, III and IV) with each other via amide functions;
α, β and γ are identical or different integers equal to 0 or 1;
b is an integer equal to 0 or to 1;
c is an integer equal to 0 or 1;
d is an integer equal to 0, 1 or 2; and if c is equal to 0 then d is equal to 1 or 2;
e is an integer equal to 0 or 1;
i and i [symbol] , whether they are identical or different, are integers less than or equal to 6 and i+i [symbol] is greater than or equal to 1 and less than or equal to 6, 1≤i+i [symbol] ≤6,
r and r [symbol] are integers equal to 0, 1, 2 or 3;
if r is equal to 0 then the hydrophobic radical according to formula I is bound to the hydrophilic backbone HB via a covalent bond between a carbonyl of the hydrophobic radical and a nitrogen atom of the hydrophilic backbone HB, thus forming an amide function resulting from the reaction of an amine function of the precursor of the hydrophilic backbone HB and an acid function borne by the precursor of the hydrophobic radical, and
if r is equal to 1, 2 or 3 then the hydrophobic radical -Hy according to formula I is bound to the hydrophilic backbone HB:
   via a covalent bond between a nitrogen atom of the hydrophobic radical and a carbonyl of the hydrophilic backbone HB, thus forming an amide function resulting from the reaction of an amine function of the precursor of the hydrophobic radical and an acid function borne by the precursor of the hydrophilic backbone HB or
   via a covalent bond between a nitrogen atom of the hydrophobic radical and a nitrogen atom of the hydrophilic backbone HB, thus forming an amide function resulting from the reaction of an acid function of the precursor of the hydrophobic radical and an amine function of the precursor of the hydrophilic backbone HB;
t is an integer equal to 0 or to 1;
B is a linear or branched alkyl radical, optionally comprising an aromatic nucleus, comprising from 1 to 9 carbon atoms, or an ether radical or unsubstituted polyether comprising from 4 to 14 carbon atoms and from 1 to 5 oxygen atoms;
Cₓ is a linear or branched monovalent alkyl radical, optionally comprising a cyclic part, wherein x indicates the number of carbon atoms and 11≤x≤25;
I [symbol] , I″ and I [symbol] , whether they are identical or different, are divalent radicals, chosen in the group consisting of a linear or branched alkyl radical, comprising from 1 to 12 carbon atoms,
I is a trivalent radical, chosen in the group consisting of a linear or branched alkyl radical, comprising from 1 to 12 carbon atoms,
Im is an imidazolyl radical,
R is a radical chosen in the group consisting of a linear or branched divalent alkyl radical, comprising from 1 to 12 carbon atoms, a branched alkyl radical of 1 to 8 carbon atoms, said alkyl radical bearing one or more free carboxylic acid function(s). a divalent, linear or branched alkyl radical comprising from 1 to 12 carbon atoms bearing one or more functions —CONH₂ or an unsubstituted ether or polyether comprising from 4 to 14 carbon atoms from 1 to 5 oxygen atoms, said free carboxylic acid functions being in the form of an alkali metal salt chosen in the group consisting of Na⁺ and K⁺, and
   when several hydrophobic radicals are borne by a hydrophilic HB backbone, then they are identical or different.

In one embodiment, the invention also relates to the precursor Hy [symbol] of the hydrophobic radical -Hy according to formula I [symbol] as defined below:

H-(GpR)ᵣ-(GpI)ᵢ-[(GpR)_{r′}-(GpI)_{i′}]ₜ-GpC Formula I

[symbol]

wherein,
GpI is a divalent radical, said radical comprising at least one imidazole Im unit according to formula III:

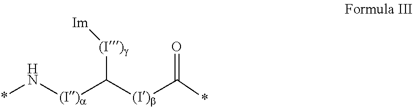

GpR is a radical according to formulas II, II [symbol] or II″:

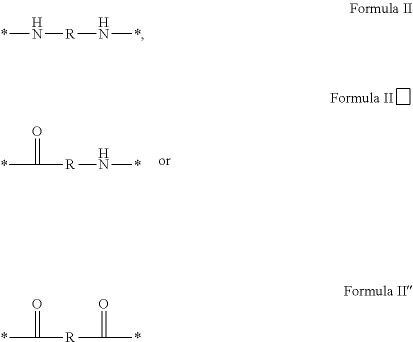

GpC is a radical according to Formula IV:

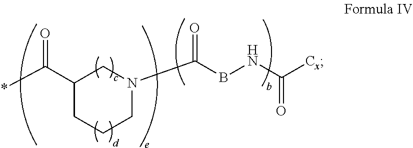

the * indicate the attachment sites of the hydrophobic radical -Hy to the hydrophilic backbone HB or the above radicals (I, II, II [symbol] II″, III and IV) with each other via amide functions;
α, β and γ are identical or different integers equal to 0 or 1;
b is an integer equal to 0 or to 1;
c is an integer equal to 0 or 1;
d is an integer equal to 0, 1 or 2; and if c is equal to 0 then d is equal to 1 or 2;
e is an integer equal to 0 or to 1;
i and i [symbol] whether they are identical or different, are integers less than or equal to 6 and i+i [symbol] is greater than or equal to 1 and less than or equal to 6, 1≤i+i [symbol] ≤6,
r and r [symbol] are integers equal to 0, 1, 2 or 3;
if r is equal to 0, then the hydrophobic radical according to formula I is bound to the hydrophilic backbone HB via a covalent bond between a carbonyl of the hydrophobic radical and a nitrogen atom of the hydrophilic backbone HB, thus forming an amide function resulting from the reaction of an amine function of the precursor of the hydrophilic backbone HB and an acid function borne by the precursor of the hydrophobic radical, and
if r is equal to 1, 2 or 3 then the hydrophobic radical -Hy according to formula I is bound to the hydrophilic backbone HB:
   via a covalent bond between a nitrogen atom of the hydrophobic radical and a carbonyl of the hydrophilic backbone HB, thus forming an amide function resulting from the reaction of an amine function of the precursor of the hydrophobic radical and an acid function borne by the precursor of the hydrophilic backbone HB or
   via a covalent bond between a nitrogen atom of the hydrophobic radical and a nitrogen atom of the hydrophilic backbone HB, thus forming an amide function resulting from the reaction of an acid function of the precursor of the hydrophobic radical and an amine function of the precursor of the hydrophilic backbone HB;
t is an integer equal to 0 or to 1;
B is a linear or branched alkyl radical, optionally comprising an aromatic nucleus, comprising from 1 to 9 carbon atoms, or a radical ether or an unsubstituted polyether comprising from 4 to 14 carbon atoms and from 1 to 5 oxygen atoms;
Cₓ is a linear or branched monovalent alkyl radical, optionally comprising a cyclic part, wherein x indicates the number of carbon atoms and 11≤x≤25;
I [symbol] I″ and I [symbol] , be they identical or different, are divalent radicals, chosen in the group consisting of a linear or branched alkyl radical, comprising from 1 to 12 carbon atoms,
I is a trivalent radical, chosen in the group consisting of a linear or branched alkyl radical, comprising from 1 to 12 carbon atoms,
Im is an imidazolyl radical,
R is a radical chosen in the group consisting of a linear or branched divalent alkyl radical, comprising from 1 to 12 carbon atoms, a branched alkyl radical of 1 to 8 carbon atoms said alkyl radical bearing one or more free carboxylic acid function(s), a divalent, linear or branched alkyl radical comprising from 1 to 12 carbon atoms bearing one or more functions —CONH₂ or a radical ether or an unsubstituted polyether comprising from 4 to 14 carbon atoms and from 1 to 5 oxygen atoms, said free carboxylic acid functions being in the form of alkaline cation salts chosen in the group consisting of Na⁺ and K⁺, and
   when several hydrophobic radicals are borne by a hydrophilic HB backbone, then they are identical or different.

In one embodiment, the invention also relates to the use of ionic species for improving the physicochemical stability of the compositions.

The amphiphilic compounds comprising a hydrophilic HB backbone bearing Formula I hydrophobic radicals are soluble in distilled water at a pH from 6 to 8, at a temperature of 25° C. and at a concentration of less than 100 mg/ml.

The invention further relates to a method of preparing stable injectable compositions.

The term [symbol] soluble [symbol] means capable of preparing a clear solution and free of particles at a concentration of less than 100 mg/ml in distilled water at 25° C.

The term [symbol] solution [symbol] means a liquid composition free from visible particles, using the procedure according to pharmacopoeias EP 8.0, point 2.9.20, and US <790>.

The term [symbol] physically stable composition [symbol] means compositions which, after a certain period of storage at a certain temperature, satisfy the criteria of visual inspection described in the European, American and International Pharmacopoeia, that is to say, compositions that are clear and that do not contain visible particles, but are also colorless.

The term [symbol] chemically stable composition [symbol] means compositions which, after storage for a certain time and at a certain temperature, exhibit minimum recovery of the active ingredients and comply with the specifications applicable to pharmaceutical products.

A traditional method for measuring the stabilities of proteins or peptides consists of measuring the formation of fibrils using Thioflavin T, also called ThT. This method makes taking measurements under conditions of temperature and stirring possible, which allows for an acceleration of the phenomenon, the latency time before the formation of fibrils, by measuring the increase in fluorescence. The compositions according to the invention have a latency time before the formation of fibrils that is markedly greater than that of glucagon at the pH of interest.

“Injectable aqueous solution” means water-based solutions that satisfy the conditions of the EP and US Pharmacopoeias, and that are liquid enough to be injected.

The term [symbol] copolyamino acid constituted by glutamic or aspartic acid units [symbol] means non-cyclic linear chains of glutamic acid or aspartic acid units bound together by peptide bonds, said chains having a C-terminal part, corresponding to the carboxylic acid of one end, and an N-terminal part, corresponding to the amine of the other end of the chain.

The term “alkyl radical” means a linear or branched carbon chain, which does not comprise a heteroatom.

The copolyamino acid is a statistical or block copolyamino acid.

The copolyamino acid is a statistical copolyamino acid in the chain of amino acid units, such as glutamic and/or aspartic or lysine and/or ornithine units.

The term hydrophilic backbone means a compound wherein the precursor (before grafting of the hydrophobic radical [symbol] Hy) has a LogP of less than 2 at pH 7.0.

According to one particular embodiment, the logP of the hydrophilic backbone precursor is less than 1 at pH 7.0.

According to one particular embodiment, the logP of the hydrophilic backbone precursor is less than 0 at pH 7.0.

The LogP or Log Kow or Partition Coefficient is a measurement of the distribution of a compound in a mixture of an immiscible solvent of n-octanol/water. LogP may be measured using the shake flask method, or when this is not possible by HPLC method (OECD Guideline for the testing of chemicals, 117, 30.03.89, Partition coefficient (n-octanol/water: HPLC method and 107, 27.07.95, Partition coefficient (n-octanol/water): Shake Flask Method). Said LogP of a compound is defined by the following equation:

logP=log(C_{oct}/Cₑₐᵤ)

wherein C_{oct} is the concentration of said compound in the n-octanol and Cwater is the concentration of said compound in water.

In the formulas, the * indicates the binding sites of the various elements represented.

In formulas I, Ia, Ib, Ic, Id, Ie and If, the * indicate the sites of attachment of the hydrophobic groups to the hydrophilic backbone HB. The -Hy radicals are attached to the hydrophilic backbone HB via amide functions.

In the Formulas II, II′ and II″, the * indicates, from left to right respectively, the GpR attachment sites:
to the hydrophilic backbone HB and
to GpI.

In Formula III, the * indicates, from left to right respectively, the GpI binding sites:
to GpR if r=1, 2 or 3 or to the hydrophilic backbone HB if r =0 and
to GpR if r [symbol] =1 or GpI if r [symbol] =0 or to GpC if t [symbol] =0.
   All the attachments between the different GpR, GpI and GpC groups are amide functions.

Each of the [symbol] Hy, GpR, GpI, GpC, and D radicals are independently identical or different from one monomer unit to another.

In one embodiment, the composition is characterized in that the ratio M between the number of hydrophobic radicals and the number of repetition units is comprised from 0.007 to 0.3.

In one embodiment, the composition is characterized in that the ratio M between the number of hydrophobic radicals and the number of repetition units is comprised from 0.01 to 0.3.

In one embodiment, the composition is characterized in that the ratio M between the number of hydrophobic radicals and the number of repetition units is comprised from 0.02 to 0.2.

In one embodiment, the composition is characterized in that the hydrophobic radical corresponds to Formula I and the ratio M between the number of hydrophobic radicals and the number of repetition units is comprised from 0.007 to 0.15.

In one embodiment, the composition is characterized in that the hydrophobic radical corresponds to Formula I and the ratio M between the number of hydrophobic radicals and the number of repetition units is comprised from 0.01 to 0.1.

In one embodiment, the composition is characterized in that the hydrophobic radical corresponds to formula I and the ratio M between the number of hydrophobic radicals and the number of repetition units is comprised from 0.02 to 0.08.

In one embodiment, the composition is characterized in that the hydrophobic radical corresponds to Formula I wherein the radical Cx comprises from 9 to 10 carbon atoms and the ratio M between the number of hydrophobic radicals and the number of repetition units is comprised from 0.03 to 0.15.

In one embodiment, the composition is characterized in that the hydrophobic radical corresponds to Formula I wherein the radical Cx comprises from 11 to 12 carbon atoms and the ratio M between the number of hydrophobic radicals and the number of repetition units is comprised from 0.015 to 0.1.

In one embodiment, the composition is characterized in that the hydrophobic radical corresponds to Formula I wherein the radical Cx comprises from 11 to 12 carbon atoms and the ratio M between the number of hydrophobic radicals and the number of repetition units is comprised from 0.02 to 0.08.

In one embodiment, the composition is characterized in that the hydrophobic radical corresponds to Formula I wherein the radical Cx comprises from 13 to 15 carbon atoms and the ratio M between the number of hydrophobic radicals and the number of repetition units is comprised from 0.01 to 0.1.

In one embodiment, the composition is characterized in that the hydrophobic radical corresponds to Formula I wherein the radical Cx comprises from 13 to 15 carbon atoms and the ratio M between the number of hydrophobic radicals and the number of repetition units is comprised from 0.01 to 0.06.

In one embodiment, the composition is characterized in that the hydrophobic radical corresponds to Formula I and the ratio M between the number of hydrophobic radicals and the number of repetition units is comprised from 0.007 to 0.3.

In one embodiment, the composition is characterized in that the hydrophobic radical corresponds to Formula I and the ratio M between the number of hydrophobic radicals and the number of repetition units is comprised from 0.01 to 0.3.

In one embodiment, the composition is characterized in that the hydrophobic radical corresponds to Formula I and the ratio M between the number of hydrophobic radicals and the number of repetition units is comprised from 0.015 to 0.2.

In one embodiment, the composition is characterized in that the hydrophobic radical corresponds to Formula I wherein the radical Cx comprises from 11 to 14 carbon atoms and the ratio M between the number of hydrophobic radicals and the number of repetition units is comprised from 0.1 to 0.2.

In one embodiment, the composition is characterized in that the hydrophobic radical corresponds to Formula I wherein the Cx radical comprises from 15 to 16 carbon atoms and the ratio M between the number of hydrophobic radicals and the number of repetition units is comprised from 0.04 to 0.15.

In one embodiment, the composition is characterized in that the hydrophobic radical corresponds to Formula I wherein the radical Cx comprises from 17 to 18 carbon atoms and the ratio M between the number of hydrophobic radicals and the number of repetition units is comprised from 0.02 to 0.06.

In one embodiment, the composition is characterized in that the hydrophobic radical corresponds to Formula I wherein the radical Cx comprises from 19 to 25 carbon atoms and the ratio M between the number of hydrophobic radicals and the number of repetition units is comprised from 0.01 to 0.06.

In one embodiment, the composition is characterized in that the hydrophobic radical corresponds to Formula I wherein the radical Cx comprises from 19 to 25 carbon atoms and the ratio M between the number of hydrophobic radicals and the number of repetition units is comprised from 0.01 to 0.05.

Amylin, or islet amyloid polypeptide (IAPP), is a 37-residue peptide hormone. It is co-secreted with insulin from pancreatic beta cells in the ratio of approximately 100:1. Amylin plays a role in glycemic regulation by stopping the secretion of endogenous glucagon and by slowing gastric emptying and by promoting satiety, thus reducing postprandial glycemic excursions in blood glucose levels.

IAPP is processed from a coding sequence of 89 residues. The Proislet amyloid polypeptide (proIAPP, proamylin, proislet protein) is produced in pancreatic beta cells (beta cells) in the form of a 67 amino acid RSO propeptide, 7404 Dalton, and undergoes post-translational modifications including the cleavage of protease to produce amylin.

In this application, amylin as mentioned refers to the compounds described in U.S. Pat. Nos. 5,124,314 and 5,234,906.

When used in reference to a peptide or protein, the term “analog” is understood to be a peptide or a protein, wherein one or more constituent amino acid residues of the primary sequence have been substituted by other amino acid residues and/or wherein one or more constituent amino acid residues have been removed and/or wherein one or more constituent amino acid residues have been added. The percentage of homology allowed for the present definition of an analogue is 50%. In the case of amylin, an analogue may for example be derived from the primary amino acid sequence of amylin by substituting one or more natural or unnatural or peptidomimetic amino acids.

When used in reference to a peptide or a protein, the term “derivative” is understood to be a peptide or a protein or an analog chemically modified by a substituent that is not present in the peptide or the protein or the reference analog, i.e., a peptide or protein that has been modified by the creation of covalent bonds, to introduce non-amino acid substituents.

An amylin receptor agonist refers to a compound that mimics one or more characteristics of amylin activity.

Amylin derivatives are described in the article Yan et al., PNAS, vol. 103, no. 7, p. 2046-2051, 2006.

In one embodiment, the substituent is chosen in the group consisting of fatty chains.

Amylin analogs are described in U.S. Pat. Nos. 5,686,411, 6,114,304 or even U.S. Pat. No. 6,410,511.

In one embodiment, amylin, the amylin receptor agonist or the amylin analog is amylin.

In one embodiment, amylin, the amylin receptor agonist or the amylin analog is an agonist at the amylin receptor.

In one embodiment, amylin, the amylin receptor agonist or the amylin analog is an amylin analogue.

In one embodiment, the composition is characterized in that the amylin analogue is pramlintide (Symlin®) marketed by the company ASTRAZENECA AB.

In one embodiment, the amphiphilic compound/amylin molar ratios, amylin receptor agonist or amylin analogue are from 1.5 to 75.

In one embodiment, the amphiphilic compound/amylin, amylin receptor agonist or amylin analogue molar ratios are comprised from 1.8 to 50.

In one embodiment, the amphiphilic compound/amylin, amylin receptor agonist or amylin analogue molar ratios are comprised from 2 to 35.

In one embodiment, the amphiphilic compound/amylin, amylin receptor agonist or amylin analogue molar ratios are comprised from 2.5 to 30.

In one embodiment, the amphiphilic compound/amylin, amylin receptor agonist or amylin analogue molar ratios are comprised from 3 to 30.

In one embodiment, the amphiphilic compound/amylin, amylin receptor agonist or amylin analogue molar ratios are comprised from 3.5 to 30.

In one embodiment, the amphiphilic compound/amylin, amylin receptor agonist or amylin analogue molar ratios are comprised from 4 to 30.

In one embodiment, the amphiphilic compound/amylin, amylin receptor agonist or amylin analogue molar ratios are comprised from 5 to 30.

In one embodiment, the amphiphilic compound/amylin, amylin receptor agonist or amylin analogue molar ratios are comprised from 7 to 30.

In one embodiment, the amphiphilic compound/amylin, amylin receptor agonist or amylin analogue molar ratios are comprised from 9 to 30.

In one embodiment, the amphiphilic compound/amylin molar ratios are comprised from 3 to 75.

In one embodiment, the amphiphilic compound/amylin molar ratios are comprised from 7 to 50.

In one embodiment, the amphiphilic compound/amylin molar ratios are comprised from 10 to 30.

In one embodiment, the amphiphilic compound/amylin molar ratios are comprised from 15 to 30.

In one embodiment, the amphiphilic compound/pramlintide molar ratios are comprised from 1.5 to 75.

In one embodiment, the amphiphilic compound/pramlintide molar ratios are comprised from 2 to 50.

In one embodiment, the amphiphilic compound/pramlintide molar ratios are comprised from 3 to 30.

In one embodiment, the amphiphilic compound/pramlintide molar ratios are comprised from 4 to 30.

In one embodiment, the amphiphilic compound/pramlintide molar ratios are comprised from 5 to 30.

In one embodiment, the amphiphilic compound/pramlintide molar ratios are comprised from 8 to 30.

In one embodiment, the amphiphilic compound/pramlintide molar ratios are comprised from 10 to 30.

In one embodiment, the hydrophobic radical Hy/amylin, amylin receptor agonist or amylin analogue molar ratios are comprised from 1.5 to 150.

In one embodiment, the hydrophobic radical Hy/amylin, amylin receptor agonist or amylin analogue molar ratios are comprised from 1.8 to 100.

In one embodiment, the hydrophobic radical Hy/amylin, amylin receptor agonist or amylin analogue molar ratios are comprised from 2 to 70.

In one embodiment, the hydrophobic radical Hy/amylin, amylin receptor agonist or amylin analogue molar ratios are comprised from 2.5 to 60.

In one embodiment, the hydrophobic radical Hy/amylin, amylin receptor agonist or amylin analogue molar ratios are comprised from 3 to 60.

In one embodiment, the hydrophobic radical Hy/amylin, amylin receptor agonist or amylin analogue molar ratios are comprised from 3.5 to 60.

In one embodiment, the hydrophobic radical Hy/amylin, amylin receptor agonist or amylin analogue molar ratios are comprised from 4 to 60.

In one embodiment, the hydrophobic radical Hy/amylin, amylin receptor agonist or amylin analogue molar ratios are comprised from 5 to 60.

In one embodiment, the hydrophobic radical Hy/amylin, amylin receptor agonist or amylin analogue molar ratios are comprised from 7 to 60.

In one embodiment, the hydrophobic radical Hy/amylin, amylin receptor agonist or amylin analogue molar ratios are comprised from 9 to 60.

In one embodiment, the hydrophobic radical Hy/amylin molar ratios are comprised from 5 to 60.

In one embodiment, the hydrophobic radical Hy/amylin molar ratios are comprised from 10 to 60.

In one embodiment, the hydrophobic radical Hy/amylin molar ratios are comprised from 15 to 60.

In one embodiment, the hydrophobic radical Hy/pramlintide molar ratios are comprised from 1.5 to 60.

In one embodiment, the hydrophobic radical Hy/pramlintide molar ratios are comprised from 2 to 60.

In one embodiment, the hydrophobic radical Hy/pramlintide molar ratios are comprised from 3 to 60.

In one embodiment, the hydrophobic radical Hy/pramlintide molar ratios are comprised from 4 to 60.

In one embodiment, the hydrophobic radical Hy/pramlintide molar ratios are comprised from 5 to 60.

In one embodiment, the hydrophobic radical Hy/pramlintide molar ratios are comprised from 8 to 60.

In one embodiment, the hydrophobic radical Hy/pramlintide molar ratios are comprised from 10 to 60.

In one embodiment, the amphiphilic compound/amylin, amylin receptor agonist or amylin analogue mass ratios are comprised from 1.0 to 70.

In one embodiment, the amphiphilic compound/amylin, amylin receptor agonist or amylin analogue mass ratios are comprised from 1.2 to 45.

In one embodiment, the amphiphilic compound/amylin, amylin receptor agonist or amylin analogue mass ratios are comprised from 1.3 to 30.

In one embodiment, the amphiphilic compound/amylin, amylin receptor agonist or amylin analogue mass ratios are comprised from 1.7 to 27.

In one embodiment, the amphiphilic compound/amylin, amylin receptor agonist or amylin analogue mass ratios are comprised from 2.0 to 27.

In one embodiment, the amphiphilic compound/amylin, amylin receptor agonist or amylin analogue mass ratios are comprised from 2.3 to 27.

In one embodiment, the amphiphilic compound/amylin, amylin receptor agonist or amylin analogue mass ratios are comprised from 2.7 to 27,

In one embodiment, the amphiphilic compound/amylin, amylin receptor agonist or amylin analogue mass ratios are comprised from 3.3 to 27.

In one embodiment, the amphiphilic compound/amylin, amylin receptor agonist or amylin analogue mass ratios are comprised from 4.7 to 27.

In one embodiment, the amphiphilic compound/amylin, amylin receptor agonist or amylin analogue mass ratios are comprised from 6.0 to 27.

In one embodiment, the amphiphilic compound/amylin mass ratios are comprised from 2.0 to 67.

In one embodiment, the amphiphilic compound/amylin mass ratios are comprised from 4.7 to 27.

In one embodiment, the amphiphilic compound/amylin mass ratios are comprised from 6.7 to 27.

In one embodiment, the amphiphilic compound/amylin mass ratios are comprised from 10 to 27.

In one embodiment, the amphiphilic compound/pramlintide mass ratios are comprised from 1.0 to 67.

In one embodiment, the amphiphilic compound/pramlintide mass ratios are comprised from 1.3 to 45.

In one embodiment, the amphiphilic compound/pramlintide mass ratios are comprised from 2.7 to 27.

In one embodiment, the amphiphilic compound/pramlintide mass ratios are comprised from 3.3 to 27.

In one embodiment, the amphiphilic compound/pramlintide mass ratios are comprised from 5.3 to 27.

In one embodiment, the amphiphilic compound/pramlintide mass ratios are comprised from 6.7 to 27.

In one embodiment, the composition is characterized in that it further comprises insulin.

In one embodiment, the composition is characterized in that the insulin is a prandial insulin. Prandial insulins are soluble at a pH of 7.

Prandial insulin is understood to be an insulin known to be fast-acting or “regular”.

The so-called fast-acting prandial insulins are insulins that must meet the needs caused by the ingestion of proteins and carbohydrates during a meal, so they must act in less than 30 min.

In one embodiment, the prandial insulin called “regular” is human insulin.

In one embodiment, prandial insulin is a recombinant human insulin as described in the European Pharmacopoeia and the American Pharmacopoeia.

Human insulin is for example marketed under the brands Humulin® (ELI LILLY) and Novolin® (NOVO NORDISK).

The so-called fast-acting prandial insulins are insulins which are obtained by recombination and whose primary sequence has been modified to reduce their time of action.

In one embodiment, the so-called fast acting prandial insulins are chosen among the group comprising insulin lispro (Humalog®), insulin glulisine (Apidra) and insulin aspart (NovoLog®).

In one embodiment, the prandial insulin is insulin lispro.

In one embodiment, the prandial insulin is insulin glulisine.

In one embodiment, the prandial insulin is insulin aspart.

The units recommended by the pharmacopoeias for insulins are presented in the table below with their corresponding mg:

| Insulin | EP Pharmacopoeia 8.0 (2014) | US Pharmacopoeia-USP38 (2015) |
|---|---|---|
| Aspart | 1U = 0.0350 mg of insulin aspart | 1 USP = 0.0350 mg of insulin aspart |
| Lispro | 1U = 0.0347 mg of insulin lispro | 1 USP = 0.0347 mg of insulin lispro |
| Human | 1UI = 0.0347 mg of human insulin | 1 USP = 0.0347 mg of human insulin |

In the case of insulin glulisine, 100U=3.49 mg of insulin glulisine (according to “Annex 1—Summary of product characteristics” relating to ADIPRA®).

However, in the remainder of the text, U is systematically and interchangeably used for the amounts and concentrations of all insulins. The respective corresponding values in mg are those given above for values expressed in U, IU or USP.

In one embodiment, it relates to a pharmaceutical formulation characterized in that the insulin concentration is comprised from 240 to 3000 μM (40 to 500 U/mL).

In one embodiment, it relates to a pharmaceutical formulation characterized in that the insulin concentration is comprised from 600 to 3000 μM (100 to 500 U/mL).

In one embodiment, it relates to a pharmaceutical formulation characterized in that the insulin concentration is comprised from 600 to 2400 μM (100 to 400 U/mL).

In one embodiment, it relates to a pharmaceutical formulation characterized in that the insulin concentration is comprised from 600 to 1800 μM (100 to 300 U/mL).

In one embodiment, it relates to a pharmaceutical formulation characterized in that the insulin concentration is comprised from 600 to 1,200 μM (100 to 200 U/mL).

In one embodiment, it relates to a pharmaceutical formulation characterized in that the insulin concentration is 600 μM (100 U/mL).

In one embodiment, it relates to a pharmaceutical formulation characterized in that the insulin concentration is 1200 μM (200 U/mL).

In one embodiment, it relates to a pharmaceutical formulation characterized in that the insulin concentration is 1800 μM (300 U/mL).

In one embodiment, it relates to a pharmaceutical formulation characterized in that the insulin concentration is 2400 μM (400 U/mL).

In one embodiment, it relates to a pharmaceutical formulation characterized in that the insulin concentration is 3000 μM (500 U/mL).

In one embodiment, comprising prandial insulin, the amphiphilic compound/amylin, amylin receptor agonist or amylin analogue molar ratios are comprised from 1.5 to 75.

In one embodiment, comprising prandial insulin, the amphiphilic compound/amylin, amylin receptor agonist or amylin analogue molar ratios are comprised from 1.8 to 50.

In one embodiment, comprising prandial insulin, the amphiphilic compound/amylin, amylin receptor agonist or amylin analogue molar ratios are comprised from 2 to 35.

In one embodiment, comprising prandial insulin, the amphiphilic compound/amylin, amylin receptor agonist or amylin analogue molar ratios are comprised from 2.5 to 30.

In one embodiment, comprising prandial insulin, the amphiphilic compound/amylin, amylin receptor agonist or amylin analogue molar ratios are comprised from 3 to 30.

In one embodiment, comprising prandial insulin, the amphiphilic compound/amylin, amylin receptor agonist or amylin analogue molar ratios are comprised from 3.5 to 30.

In one embodiment, comprising prandial insulin, the amphiphilic compound/amylin, amylin receptor agonist or amylin analogue molar ratios are comprised from 4 to 30.

In one embodiment, comprising prandial insulin, the amphiphilic compound/amylin, amylin receptor agonist or amylin analogue molar ratios are comprised from 5 to 30.

In one embodiment, comprising prandial insulin, the amphiphilic compound/amylin, amylin receptor agonist or amylin analogue molar ratios are comprised from 7 to 30.

In one embodiment, comprising prandial insulin, the amphiphilic compound/amylin, amylin receptor agonist or amylin analogue molar ratios are comprised from 9 to 30.

In one embodiment, comprising prandial insulin, the amphiphilic compound/amylin molar ratios are comprised from 5 to 75.

In one embodiment, comprising prandial insulin, the amphiphilic compound/amylin molar ratios are comprised from 10 to 50.

In one embodiment, comprising prandial insulin, the amphiphilic compound/amylin molar ratios are comprised from 15 to 30.

In one embodiment, comprising prandial insulin, the amphiphilic compound/pramlintide molar ratios are comprised from 1.5 to 75.

In one embodiment, comprising prandial insulin, the amphiphilic compound/pramlintide molar ratios are comprised from 2 to 50.

In one embodiment, comprising prandial insulin, the amphiphilic compound/pramlintide molar ratios are comprised from 3 to 30.

In one embodiment, comprising prandial insulin, the amphiphilic compound/pramlintide molar ratios are comprised from 4 to 30.

In one embodiment, comprising prandial insulin, the amphiphilic compound/pramlintide molar ratios are comprised from 5 to 30.

In one embodiment, comprising prandial insulin, the amphiphilic compound/pramlintide molar ratios are comprised from 8 to 30.

In one embodiment, comprising prandial insulin, the amphiphilic compound/pramlintide molar ratios are comprised from 10 to 30.

In one embodiment, comprising prandial insulin, the hydrophobic radical [symbol] Hy/amylin, amylin receptor agonist or amylin analogue molar ratios are comprised from 1.5 to 150.

In one embodiment, comprising prandial insulin, the hydrophobic radical [symbol] Hy/amylin, amylin receptor agonist or amylin analogue molar ratios are comprised from 1.8 to 100.

In one embodiment, comprising prandial insulin, the hydrophobic radical [symbol] Hy/amylin, amylin receptor agonist or amylin analogue molar ratios are comprised from 2 to 70.

In one embodiment, comprising prandial insulin, the hydrophobic radical [symbol] Hy/amylin, amylin receptor agonist or amylin analogue molar ratios are comprised from 2.5 to 60.

In one embodiment, comprising prandial insulin, the hydrophobic radical [symbol] Hy/amylin, amylin receptor agonist or amylin analogue molar ratios are comprised from 3 to 60.

In one embodiment, comprising prandial insulin, the hydrophobic radical [symbol] Hy/amylin, amylin receptor agonist or amylin analogue molar ratios are comprised from 3.5 to 60.

In one embodiment, comprising prandial insulin, the hydrophobic radical [symbol] Hy/amylin, amylin receptor agonist or amylin analogue molar ratios are comprised from 4 to 60.

In one embodiment, comprising prandial insulin, the hydrophobic radical [symbol] Hy/amylin, amylin receptor agonist or amylin analogue molar ratios are comprised from 5 to 60.

In one embodiment, comprising prandial insulin, the hydrophobic radical [symbol] Hy/amylin, amylin receptor agonist or amylin analogue molar ratios are comprised from 7 to 60.

In one embodiment, comprising prandial insulin, the hydrophobic radical [symbol] Hy/amylin, amylin receptor agonist or amylin analogue molar ratios are comprised from 9 to 60.

In one embodiment, comprising prandial insulin, the hydrophobic radical [symbol] Hy/amylin molar ratios are comprised from 5 to 60.

In one embodiment, comprising prandial insulin, the hydrophobic radical [symbol] Hy/amylin molar ratios are comprised from 10 to 60.

In one embodiment, comprising prandial insulin, the hydrophobic radical [symbol] Hy/amylin molar ratios are comprised from 15 to 60.

In one embodiment, comprising prandial insulin, the hydrophobic radical [symbol] Hy/pramlintide molar ratios are comprised from 1.5 to 60.

In one embodiment, comprising prandial insulin, the hydrophobic radical [symbol] Hy/pramlintide molar ratios are comprised from 2 to 60.

In one embodiment, comprising prandial insulin, the hydrophobic radical [symbol] Hy/pramlintide molar ratios are comprised from 3 to 60.

In one embodiment, comprising prandial insulin, the hydrophobic radical [symbol] Hy/pramlintide molar ratios are comprised from 4 to 60.

In one embodiment, comprising prandial insulin, the hydrophobic radical [symbol] Hy/pramlintide molar ratios are comprised from 5 to 60.

In one embodiment, comprising prandial insulin, the hydrophobic radical [symbol] Hy/pramlintide molar ratios are comprised from 8 to 60.

In one embodiment, comprising prandial insulin, the hydrophobic radical [symbol] Hy/pramlintide molar ratios are comprised from 10 to 60.

In one embodiment, comprising prandial insulin, the amphiphilic compound/amylin, amylin receptor agonist or amylin analogue mass ratios are comprised from 1.0 to 70.

In one embodiment, comprising prandial insulin, the amphiphilic compound/amylin, amylin receptor agonist or amylin analogue mass ratios are comprised from 1.2 to 45.

In one embodiment, comprising prandial insulin, the amphiphilic compound/amylin, amylin receptor agonist or amylin analogue mass ratios are comprised from 1.3 to 30.

In one embodiment, comprising prandial insulin, the amphiphilic compound/amylin, amylin receptor agonist or amylin analogue mass ratios are comprised from 1.7 to 27.

In one embodiment, comprising prandial insulin, the amphiphilic compound/amylin, amylin receptor agonist or amylin analogue mass ratios are comprised from 2.0 to 27.

In one embodiment, comprising prandial insulin, the amphiphilic compound/amylin, amylin receptor agonist or amylin analogue mass ratios are comprised from 2.3 to 27.

In one embodiment, comprising prandial insulin, the amphiphilic compound/amylin, amylin receptor agonist or amylin analogue mass ratios are comprised from 2.7 to 27.

In one embodiment, comprising prandial insulin, the amphiphilic compound/amylin, amylin receptor agonist or amylin analogue mass ratios are comprised from 3.3 to 27.

In one embodiment, comprising prandial insulin, the amphiphilic compound/amylin, amylin receptor agonist or amylin analogue mass ratios are comprised from 4.7 to 27.

In one embodiment, comprising prandial insulin, the amphiphilic compound/amylin, amylin receptor agonist or amylin analogue mass ratios are comprised from 6.0 to 27.

In one embodiment, comprising prandial insulin, the amphiphilic compound/amylin mass ratios are comprised from 3.3 to 67.

In one embodiment, comprising prandial insulin, the amphiphilic compound/amylin mass ratios are comprised from 6.6 to 27.

In one embodiment, comprising prandial insulin, the amphiphilic compound/amylin mass ratios are comprised from 10 to 27.

In one embodiment, comprising prandial insulin, the amphiphilic compound/pramlintide mass ratios are comprised from 1.0 to 67.

In one embodiment, comprising prandial insulin, the amphiphilic compound/pramlintide mass ratios are comprised from 1.2 to 45.

In one embodiment, comprising prandial insulin, the amphiphilic compound/pramlintide mass ratios are comprised from 1.3 to 27.

In one embodiment, comprising prandial insulin, the amphiphilic compound/pramlintide mass ratios are comprised from 1.7 to 27.

In one embodiment, comprising prandial insulin, the amphiphilic compound/pramlintide mass ratios are comprised from 2.0 to 27.

In one embodiment, comprising prandial insulin, the amphiphilic compound/pramlintide mass ratios are comprised from 2.3 to 27.

In one embodiment, comprising prandial insulin, the amphiphilic compound/pramlintide mass ratios are comprised from 2.7 to 27.

In one embodiment, comprising prandial insulin, the amphiphilic compound/pramlintide mass ratios are comprised from 3.3 to 27.

In one embodiment, comprising prandial insulin, the amphiphilic compound/pramlintide mass ratios are comprised from 4.7 to 27.

In one embodiment, comprising prandial insulin, the amphiphilic compound/pramlintide mass ratios are comprised from 6.0 to 27.

In one embodiment, the composition comprises amylin, an amylin receptor agonist or an amylin analog, in combination or not with a prandial insulin, with GLP-1, GLP-1 analogues, GLP-1 receptor agonists, commonly referred to as GLP-1 RA and an amphiphilic compound comprising a hydrophilic backbone HB, substituted by at least one hydrophobic radical

In addition, it is particularly interesting to combine amylin, an amylin receptor agonist or an amylin analogue, in combination or not with a prandial insulin, with GLP-1, GLP-1 analogues, GLP-1 receptor agonists, these are commonly referred to as GLP-1 RA. Specifically, this makes it possible to potentiate the effect of insulin and is recommended in some types of diabetes treatment.

In one embodiment, the GLP-1, GLP-1 analogues, or GLP-1 RA are called “fast-acting”. “Fast-acting” means GLP-1, GLP-1 analogues, or GLP-1 RA, whose apparent elimination half-life after subcutaneous injection in humans is less than 8 hours, in particular less than 5 hours, preferably less than 4 hours or even less than 3 hours, such as, for example, exenatide and lixisenatide.

In one embodiment, the GLP-1, GLP-1 analogues, or GLP-1 RAs are chosen in the group consisting of exenatide or Byetta® (ASTRA-ZENECA), lixisenatide or Lyxumia® (SANOFI), their analogues or derivatives and their pharmaceutically acceptable salts.

In one embodiment, the GLP-1, GLP-1 analogue, or GLP-1 RA is exenatide or Byetta®, its analogues or derivatives and their pharmaceutically acceptable salts.

In one embodiment, the GLP-1, GLP-1 analogue, or GLP-1 RA is lixisenatide or Lyxumia , their analogues or derivatives and their pharmaceutically acceptable salts.

In one embodiment, the exenatide concentration, their analogues or derivatives and their pharmaceutically acceptable salts is within the range of 0.01 to 1.0 mg per 1 mg of an amylin receptor agonist or an amylin analog.

In one embodiment, the concentration of exenatide, their analogues or derivatives thereof and their pharmaceutically acceptable salts is comprised from 0.01 to 0.5 mg per 1 mg of an amylin receptor agonist or an amylin analog.

In one embodiment, the concentration of exenatide, their analogues or derivatives thereof and their pharmaceutically acceptable salts is comprised from 0.02 to 0,4 mg per 1 mg of an amylin receptor agonist or an amylin analog.

In one embodiment, the concentration of exenatide, their analogues or derivatives thereof and their pharmaceutically acceptable salts is comprised from 0.03 to 0,3 mg per 1 mg of an amylin receptor agonist or an amylin analog.

In one embodiment, the concentration of exenatide, their analogues or derivatives thereof and their pharmaceutically acceptable salts is comprised from 0.04 to 0,2 mg per 1 mg of an amylin receptor agonist or an amylin analog.

In one embodiment, the concentration of exenatide, their analogues or derivatives thereof and their pharmaceutically acceptable salts is comprised from 0.04 to 0,15 mg per 1 mg of an amylin receptor agonist or an amylin analog.

In one embodiment, the concentration of lixisenatide, their analogues or derivatives thereof and their pharmaceutically acceptable salts is comprised from 0.01 to 1 mg per 1 mg of the amylin receptor agonist or an amylin analog.

In one embodiment, the concentration of lixisenatide, their analogues or derivatives thereof and their pharmaceutically acceptable salts is comprised from 0.01 to 0.5 mg per 1 mg of an amylin receptor agonist or an amylin analog.

In one embodiment, the concentration of lixisenatide, their analogues or derivatives thereof and their pharmaceutically acceptable salts is comprised from 0.02 to 0,4 mg per 1 mg of an amylin receptor agonist or an amylin analog.

In one embodiment, the concentration of lixisenatide, their analogues or derivatives thereof and their pharmaceutically acceptable salts is comprised from 0.03 to 0,3 mg per 1 mg of an amylin receptor agonist or an amylin analog.

In one embodiment, the concentration of lixisenatide, their analogues or derivatives thereof and their pharmaceutically acceptable salts is comprised from 0.04 to 0,2 mg per 1 mg of an amylin receptor agonist or an amylin analog.

In one embodiment, the concentration of lixisenatide, their analogues or derivatives thereof and their pharmaceutically acceptable salts is comprised from 0.04 to 0.15 mg per 1 mg of an amylin receptor agonist or an amylin analog.

In one embodiment, the compositions according to the invention are produced by mixing solutions of amylin and commercial solutions of GLP-1, GLP-1 analogue, or GLP-1 receptor agonist RA in volume ratios within a range of 10/90 to 90/10 in the presence of an amphiphilic compound.

In one embodiment, said at least one ionic species allows for improved stability of the compositions.

In one embodiment, said at least one ionic species is chosen among cations that are at least divalent, anions, cations or zwitterions and mixtures thereof.

In one embodiment, the at least divalent cation salt is an inorganic cation salt chosen among the group of the at least divalent cations derived from metals such as zinc or from alkaline earth metals such as magnesium or calcium.

In one embodiment, the at least divalent cation salt is a zinc salt.

In one embodiment, the at least divalent cation salt is a calcium salt.

In one embodiment, the at least divalent cation salt is a magnesium salt.

In one embodiment, the at least divalent cation salts are added to the composition in the form of salts chosen among chlorides, phosphates, sulphates or hydroxides.

In one embodiment, the at least divalent cation salts are present at a concentration from 0.1 to 5 mM.

In one embodiment, the at least divalent cation salts are present at a concentration from 0.2 to 4 mM.

In one embodiment, the at least divalent cation salts the at least divalent cation salts are present at a concentration from 0.5 to 3 mM.

In one embodiment, the at least divalent cation salts are present at a concentration of from 0.1 to 5 mM per 1 mg/ml of amylin, amylin receptor agonist or amylin analog.

In one embodiment, the at least divalent cation salts are present at a concentration of from 0.2 to 4 mM per 1 mg of amylin, amylin receptor agonist or amylin analog.

In one embodiment, the at least divalent cation salts are present at a concentration of from 0.5 to 3 mM per 1 mg of amylin, amylin receptor agonist or amylin analog.

In one embodiment, zinc salts are present at a concentration from 0.1 to 5 mM,

In one embodiment, zinc salts are present at a concentration from 0.2 to 4 mM.

In one embodiment, the zinc salts are present at a concentration from 0.5 to 3 mM.

In one embodiment, zinc salts are present at a concentration of from 0.1 to 5 mM per 1 mg of amylin, amylin receptor agonist or amylin analog.

In one embodiment, zinc salts are present at a concentration of from 0.2 to 4 mM per 1 mg of amylin, amylin receptor agonist or amylin analog.

In one embodiment, zinc salts are present at a concentration of from 0.5 to 3 mM per 1 mg of amylin, amylin receptor agonist or amylin analog.

In one embodiment, said at least one ionic species is chosen among anions, cations or zwitterions that are different from the at least divalent cations.

In one embodiment, ionic species contain less than 10 carbon atoms.

Said ionic species are chosen in the group consisting of the group of anions, cations and/or zwitterions. Zwitterion means a species bearing at least one positive charge and at least one negative charge on two non-adjacent atoms.

Said ionic species are used alone or in a mixture and preferably in a mixture.

In one embodiment, anions are chosen in the group consisting of organic anions.

In one embodiment, organic ionic species comprise less than 10 carbon atoms.

In one embodiment, organic anions are chosen in the group consisting of acetate, citrate and succinate.

In one embodiment, anions are chosen among inorganic anions.

In one embodiment, the inorganic anions are chosen in the group consisting of sulfates, phosphates and halides, in particular, chloride ions.

In one embodiment, the inorganic anions are chosen among chloride ions.

In one embodiment, chloride ions are added in the form of sodium chloride salt.

In one embodiment, the composition comprises sodium chloride.

In one embodiment, cations are chosen among organic cations.

In one embodiment, organic cations comprise less than 10 carbon atoms.

In one embodiment, organic cations are chosen in the group consisting of ammoniums, for example 2-amino-2-(hydroxymethyl) propane-1,3-diol where the amine is in the form of ammonium.

In one embodiment, the cations are chosen among monovalent inorganic cations.

In one embodiment, the inorganic cationsare chosen in the group consisting of cations derived from alkali metals, in particular Na⁺ and K⁺,

In one embodiment, the zwitterions are chosen among organic zwitterions.

In one embodiment, organic zwitterions are chosen among amino acids.

In one embodiment, the amino acids are chosen among aliphatic amino acids in the group consisting of glycine, alanine, valine, isoleucine and leucine.

In one embodiment, the amino acids are chosen among cyclic amino acids in the group consisting of proline.

In one embodiment, the amino acids are chosen among hydroxylated or sulfur-containing amino acids in the group consisting of cysteine, serine, threonine, and methionine.

In one embodiment, the amino acids are chosen among aromatic amino acids in the group consisting of phenylalanine, tyrosine and tryptophan.

In one embodiment, the amino acids are chosen among amino acids wherein the carboxyl function of the side chain is amidified in the group consisting of asparagine and glutamine.

In one embodiment, organic zwitterions are chosen in the group consisting of amino acids having an uncharged side chain.

In one embodiment, organic zwitterions are chosen in the group consisting of amino diacids or acidic amino acids.

In one embodiment, the amino diacids are in the group chosen in the group consisting of glutamic acid and aspartic acid, optionally in the form of salts.

In one embodiment, organic zwitterions are chosen in the group consisting of basic or so-called [symbol] cationic [symbol] amino acids.

In one embodiment, the so-called “cationic” amino acids are chosen among arginine, histidine and lysine, in particular arginine and lysine.

In particular, zwitterions include as many negative charges as positive charges and therefore a zero overall charge at the isoelectric point and/or at a pH from 6 to 8.

Said ionic species are introduced into the compositions in the form of salts. These may be introduced into the compositions in solid form before they dissolve, or in solution form, in particular in the case of a concentrated solution.

For example, the inorganic cations are provided in the form of salts chosen among sodium chloride, sodium phosphate and sodium sulfate.

For example, organic anions are provided in the form of salts chosen among sodium or potassium citrate, sodium acetate.

For example, amino acids are added in the form of salts. chosen among arginine hydrochloride, histidine hydrochloride or in unsalted form such as, for example, histidine, arginine.

In one embodiment, said at least one ionic species is a combination of a divalent cation and an inorganic anion.

In one embodiment, said at least one ionic species is a combination of a divalent cation and chloride ions.

In one embodiment, said at least one ionic species is a combination of a zinc salt and chloride ions.

In one embodiment, said at least one ionic species is a combination of a zinc salt and sodium chloride salt.

In one embodiment, the total molar concentration of ionic species in the composition is greater than or equal to 10 mM.

In one embodiment, the total molar concentration of ionic species in the composition is greater than or equal to 20 mM.

In one embodiment, the total molar concentration of ionic species in the composition is greater than or equal to 40 mM.

In one embodiment, the total molar concentration of ionic species in the composition is greater than or equal to 50 mM.

In one embodiment, the total molar concentration in ionic species in the composition is lower than or equal to 250 mM.

In one embodiment, the total molar concentration in ionic species in the composition is lower than or equal to 200 mM.

In one embodiment, the total molar concentration in ionic species in the composition is lower than or equal to 150 mM.

In one embodiment, the total molar concentration in ionic species in the composition is lower than or equal to 100 mM.

In one embodiment, the total molar concentration in ionic species in the composition is lower than or equal to 75 mM.

In one embodiment, the total molar concentration in ionic species in the composition is lower than or equal to 50 mM.

In one embodiment, the total molar concentration in ionic species in the composition is comprised from 10 to 250 mM.

In one embodiment, the total molar concentration in ionic species in the composition is comprised from 20 to 200 mM.

In one embodiment, the total molar concentration in ionic species in the composition is comprised from 25 to 150 mM.

In one embodiment, the total molar concentration in ionic species in the composition is comprised from 50 to 100 mM.

In one embodiment, the total molar concentration in chloride ions in the composition is greater than or equal to 10 mM.

In one embodiment, the total molar concentration in chloride ions in the composition is greater than or equal to 20 mM.

In one embodiment, the total molar concentration in chloride ions in the composition is greater than or equal to 40 mM.

In one embodiment, the total molar concentration in chloride ions in the composition is greater than or equal to 50 mM.

In one embodiment, the molar concentration in chloride ions in the composition is less than or equal to 250 mM.

In one embodiment, the molar concentration in chloride ions in the composition is less than or equal to 200 mM.

In one embodiment, the molar concentration in chloride ions in the composition is less than or equal to 150 mM.

In one embodiment, the molar concentration in chloride ions in the composition is less than or equal to 100 mM.

In one embodiment, the molar concentration in chloride ions in the composition is less than or equal to 75 mM.

In one embodiment, the molar concentration in chloride ions in the composition is less than or equal to 50 mM.

In one embodiment, the molar concentration in chloride ions in the composition is comprised from 10 to 250 mM.

In one embodiment, the molar concentration in chloride ions in the composition is comprised from 20 to 200 mM.

In one embodiment, the molar concentration in chloride ions in the composition is comprised from 25 to 150 mM.

In one embodiment, the molar concentration in chloride ions in the composition is comprised from 50 to 100 mM.

In one embodiment, the molar concentration in chloride ions in the composition is comprised from 30 to 300 mM.

In one embodiment, the molar concentration in chloride ions in the composition is comprised from 50 to 250 mM.

In one embodiment, the molar concentration in chloride ions in the composition is comprised from 80 to 220 mM.

In one embodiment, the molar concentration in chloride ions in the composition is comprised from 100 to 200 mM.

In one embodiment, the composition comprises from 10 to 500 mM of NaCl.

In one embodiment, the composition comprises from 15 to 400 mM of NaCl.

In one embodiment, the composition comprises from 20 to 300 mM of NaCl.

In one embodiment, the composition comprises from 25 to 200 mM of NaCl.

In one embodiment, the composition comprises from 50 to 100 mM of NaCl.

In one embodiment, the compositions according to the invention further comprise buffers.

In one embodiment, the compositions according to the invention comprise buffers at a concentration of from 0 to 100 mM.

In one embodiment, the compositions according to the invention comprise buffers at a concentration of from 15 to 50 mM.

In one embodiment, the compositions according to the invention comprise a buffer chosen in the group consisting of a phosphate buffer and Tris (trishydroxymethylaminomethane).

In one embodiment, the buffer is sodium phosphate.

In one embodiment, the buffer is Tris (trishydroxymethylaminomethane).

In one embodiment, the compositions according to the invention further comprise preservatives.

In one embodiment, the preservatives are chosen in the group consisting of m-cresol and phenol, alone or as a mixture.

In one embodiment, the concentration of preservatives is comprised from 10 to 50 mM.

In one embodiment, the concentration of preservatives is comprised from 10 to 40 mM.

In one embodiment, the compositions according to the invention further comprise a surfactant.

In one embodiment, the surfactant is chosen in the group consisting of propylene glycol and polysorbate.

The compositions according to the invention may further comprise additives such as tonicity agents.

In one embodiment, the tonicity agents are chosen in the group consisting of glycerin, mannitol and glycine.

The compositions according to the invention may further comprise all excipients compatible with pharmacopoeia and compatible with insulins used at the customary concentrations.

The invention also relates to a pharmaceutical formulation according to the invention, characterized in that it is obtained by drying and/or freeze drying.

In the case of local and systemic releases, the proposed modes of administration are intravenous, subcutaneous, intradermal or intramuscular.

Transdermal, oral, nasal, vaginal, ocular, oral, and pulmonary routes of administration are also considered.

The invention also relates to an implantable or transportable pump, comprising a composition according to the invention.

The invention also relates to the use of a composition according to the invention intended to be placed in an implantable or transportable pump.

The invention also relates to single-dose formulations at a pH comprised from 6.0 to 8.0, comprising amylin, an amylin receptor agonist or an amylin analogue and an amphiphilic composition according to the invention.

The invention also relates to single-dose formulations at a pH comprised from 6.0 to 8.0, comprising amylin, an amylin receptor agonist or an amylin analogue, an amphiphilic composition according to the invention and a GLP-1, a GLP-1 analogue or a GLP-1 RA, as defined above.

The invention also relates to single-dose formulations at a pH comprised from 6.6 to 7.8, comprising amylin, an amylin receptor agonist or an amylin analogue and an amphiphilic composition according to the invention.

The invention also relates to single-dose Formulations at a pH comprised from 6.6 to 7.8, comprising amylin, an amylin receptor agonist or an amylin analogue, an amphiphilic composition according to the invention and a prandial insulin, as defined above.

The invention also relates to single-dose Formulations at a pH comprised from 6.6 to 7.6, comprising amylin, an amylin receptor agonist or an amylin analogue and an amphiphilic composition according to the invention.

The invention also relates to single-dose Formulations at a pH comprised from 6.6 to 7.6, comprising amylin, an amylin receptor agonist or an amylin analogue, an amphiphilic composition according to the invention and a prandial insulin, as defined above.

In one embodiment, the single-dose Formulations further comprise an amphiphilic composition as defined above.

In one embodiment, the Formulations are in the form of an injectable solution.

The preparation of a composition according to the invention has the advantage of being able to be carried out by simple mixing of an aqueous amylin solution, an amylin receptor agonist or an amylin analogue, and an amphiphilic composition comprising a hydrophilic backbone HB bearing a hydrophobic agent according to the invention, in an aqueous solution or in freeze dried form. If necessary, the pH of the preparation is adjusted to a pH of from 6 to 8.

The preparation of a composition according to the invention has the advantage of being able to be carried out by simple mixing of an aqueous amylin solution, an amylin receptor agonist or an amylin analogue, prandial insulin, and an amphiphilic composition comprising a hydrophilic backbone HB bearer of at least one hydrophobic radical according to the invention, in an aqueous solution or in freeze dried form. If necessary, the pH of the preparation is adjusted to a pH comprised from 6 to 8.

In one embodiment, the mixture of prandial insulin and amphiphilic composition is concentrated by ultrafiltration.

If necessary, the composition of the mixture is adjusted with excipients such as glycerin, m-cresol, zinc chloride, and polysorbate (Tween®) by adding concentrated solutions of these excipients in the mixture. If necessary, the pH of the preparation is adjusted to a pH of from 6 to 8.

In one embodiment, the compositions are characterized in that said compositions exhibit a stability measured by ThT greater than that of a reference composition comprising amylin, an amylin receptor agonist or an amylin analogue, but not comprising an amphiphilic composition bearing hydrophobic radicals -Hy.

In one embodiment, the compositions are characterized in that said compositions exhibit a stability measured by ThT greater than that of a reference composition comprising amylin, an amylin receptor agonist or an amylin analogue, in combination with an insulin, but not comprising an amphiphilic composition bearing hydrophobic radicals [symbol] Hy.

In one embodiment, the compositions are characterized in that said compositions exhibit a stability measured by ThT greater than that of a reference composition comprising amylin, an amylin receptor agonist or an amylin analogue, in combination with a GLP-1, a GLP-1 analogue or a GLP-1 receptor agonist, but not comprising an amphiphilic composition bearing hydrophobic radicals -Hy.

In one embodiment, the compositions are characterized in that said compositions exhibit a stability measured by ThT greater than that of a reference composition comprising amylin, an amylin receptor agonist or an amylin analogue, in combination with an insulin and a GLP-1, a GLP-1 analogue or a GLP-1 receptor agonist, but not comprising an amphiphilic composition bearing hydrophobic radicals -Hy.

The invention also relates to said amphiphilic compositions bearing hydrophobic radicals of Formula I and the precursors of said hydrophobic radicals.

In one embodiment, the invention also relates to the precursors of said Formula I hydrophobic radicals.

The invention also relates to a use of an amphiphilic composition bearing hydrophobic radicals -Hy to stabilize a composition comprising amylin, an amylin receptor agonist or an amylin analogue,

The invention also relates to a use of an amphiphilic composition bearing hydrophobic radicals -Hy to stabilize a composition comprising amylin, an amylin receptor agonist or an amylin analogue, and a prandial insulin, and optionally a GLP-1, a GLP-1 analogue or a GLP-1 receptor agonist,

The invention relates to a method to stabilize a composition comprising amylin, an amylin receptor agonist or an amylin analogue or a method to stabilize a composition comprising amylin, an amylin receptor agonist or an amylin analogue, and a prandial insulin, and optionally a GLP-1, a GLP-1 analogue or a GLP-1 receptor agonist,

In one embodiment, the composition according to the invention is characterized in that the copolyamino acid is obtained from a polyamino acid obtained by polymerization.

In one embodiment, the composition according to the invention is characterized in that the copolyamino acid is obtained from a polyamino acid obtained by polymerization by ring opening of a derivative of N-carboxyanhydride of glutamic acid or of an aspartic acid N-carboxyanhydride derivative.

In one embodiment, the composition according to the invention is characterized in that the copolyamino acid is obtained from a polyamino acid obtained by polymerization of a derivative of a glutamic acid N-carboxyanhydride or of an aspartic acid N-carboxyanhydride derivative as described in the Article by Deming, T. J., Adv. Polym. Sci. 2006, 202, 1-18.

In one embodiment, the composition according to the invention is characterized in that the copolyamino acid is obtained from a polyamino acid obtained by polymerization of a derivative of a glutamic acid N-carboxyanhydride.

In one embodiment, the composition according to the invention is characterized in that the copolyamino acid is obtained from a polyamino acid obtained by polymerization of a derivative of a glutamic acid N-carboxyanhydride chosen in the group consisting of N-carboxyanhydride poly-methyl glutamate (GluOMe-NCA), N-carboxyanhydride poly-glutamate benzyl (GluOBzl-NCA) and N-carboxyanhydride t-butyl poly-glutamate (GluOtBu-NCA).

In one embodiment, the glutamic acid N-carboxyanhydride derivative is poly-methyl L-glutamate N-carboxyanhydride (L-GluOMe-NCA).

In one embodiment, the glutamic acid N-carboxyanhydride derivative is poly-benzyl L-glutamate N-carboxyanhydride (L-GluOMe-NCA).

In one embodiment, the composition according to the invention is characterized in that the copolyamino acid is obtained from a polyamino acid obtained by polymerization of a derivative of a glutamic acid N-carboxyanhydride or of an aspartic acid N-carboxyanhydride derivative using as initiator an organometallic complex of a transition metal as described in the publication by Deming, T. J., Nature 1997, 390, 386-389.

In one embodiment, the composition according to the invention is characterized in that the copolyamino acid is obtained from a polyamino acid obtained by polymerization of a derivative of a glutamic acid N-carboxyanhydride or of an aspartic acid N-carboxyanhydride derivative using ammonia or a primary amine as initiator as described in the patent FR 2,801,226 and the references cited in this patent. Likewise, the initiator may be a polyamine in order to obtain polyamino acid comprising several PLGs. Said polyamines may be chosen among diamines, triamines and tetramines. The amines of these polyamines may be primary amines.

In one embodiment, the composition according to the invention is characterized in that the copolyamino acid is obtained from a polyamino acid obtained by polymerization of a derivative of a glutamic acid N-carboxyanhydride or of an aspartic acid N-carboxyanhydride derivative using hexamethyldisilazane as initiator as described in the publication by Lu H., et al., J. Am. Chem. Soc. 2007, 129, 14114-14115 or a silylated amine as described in the publication by Lu H., et al., J. Am. Chem. Soc. 2008, 130, 12562-12563.

In one embodiment, the composition according to the invention is characterized in that the process for the synthesis of the polyamino acid obtained by polymerization of a derivative of N-carboxyanhydride of glutamic acid of an aspartic acid N-carboxyanhydride derivative from which the copolyamino acid is derived, comprises an ester function hydrolysis step.

In one embodiment, this ester function hydrolysis step may consist of hydrolysis in an acidic medium or hydrolysis in a basic medium or may be carried out by hydrogenation.

In one embodiment, this ester group hydrolysis step is hydrolysis in an acidic medium.

In one embodiment, this ester group hydrolysis step is carried out by hydrogenation.

In one embodiment, the composition according to the invention is characterized in that the copolyamino acid is obtained from a polyamino acid obtained by depolymerization of a higher molecular weight polyamino acid of higher molecular weight.

In one embodiment, the composition according to the invention is characterized in that the copolyamino acid is obtained from a polyamino acid obtained by enzymatic depolymerization of a higher molecular weight polyamino acid.

In one embodiment, the composition according to the invention is characterized in that the copolyamino acid is obtained from a polyamino acid obtained by chemical depolymerization of a higher molecular weight polyamino acid.

In one embodiment, the composition according to the invention is characterized in that the copolyamino acid is obtained from a polyamino acid obtained by enzymatic depolymerization of a higher molecular weight polyamino acid.

In one embodiment, the composition according to the invention is characterized in that the copolyamino acid is obtained from a polyamino acid obtained by depolymerization of a higher molecular weight polyamino acid, chosen in the group consisting of sodium polyglutamate and sodium polyaspartate.

In one embodiment, the composition according to the invention is characterized in that the copolyamino acid is obtained from a polyamino acid obtained by depolymerization of a higher molecular weight polyamino acid.

In one embodiment, the composition according to the invention is characterized in that the copolyamino acid is obtained from a polyamino acid obtained by depolymerization of a higher molecular weight sodium polyaspartate.

In one embodiment, the composition according to the invention is characterized in that the copolyamino acid is obtained by grafting a hydrophobic group onto a poly-L-glutamic acid or poly-L-aspartic acid using the amide bond formation methods well known to those skilled in the art.

In one embodiment, the composition according to the invention is characterized in that the copolyamino acid is obtained by grafting a hydrophobic group onto a poly-L-glutamic acid or poly-L-aspartic acid using the amide bond formation methods used for peptide synthesis. In one embodiment, the composition according to the invention is characterized in that the copolyamino acid is obtained by grafting a hydrophobic group onto a poly-L-glutamic acid or a poly-L-aspartic acid as described in patent FR 2,840,614.

During the synthesis of the intermediate compositions Hy and during grafting, traditional protection and deprotection techniques are used:
the one or more free carboxylic acid function(s) of Hy may be in protected form before grafting on the PLG via an acid-protecting group. For example, this protection is accomplished by esterification using methanol, ethanol, benzyl alcohol or t-Butanol. After grafting, the functions are deprotected, i.e., a deprotection reaction is carried out so that the carboxylic function(s) is (are) free or in form of an alkaline cation salt chosen in the group consisting of Na⁺ and K⁺.
the one or more amine function(s) may be in protected form before grafting to the PLG via an amine protective group. For example, this protection is performed by acid or basic hydrolysis under heat via the phenylmethoxy carbonyl group or the 1,1-dimethylethoxy carbonyl group. After grafting, the functions are deprotected, i.e., deprotection reaction is carried out so that the amine function(s) is (are) free.
one or more free amine function(s) of the imidazole Hyd may be in protected form before grafting onto the PLG via an amine protective group. For example, this protection is carried out by a nucleophilic substitution in a basic medium via the benzyloxymethyl (BOM) or trityl (Tr) group. After grafting, the functions are deprotected, i.e., a deprotection reaction is carried out so that the amine function(s) is (are) free.

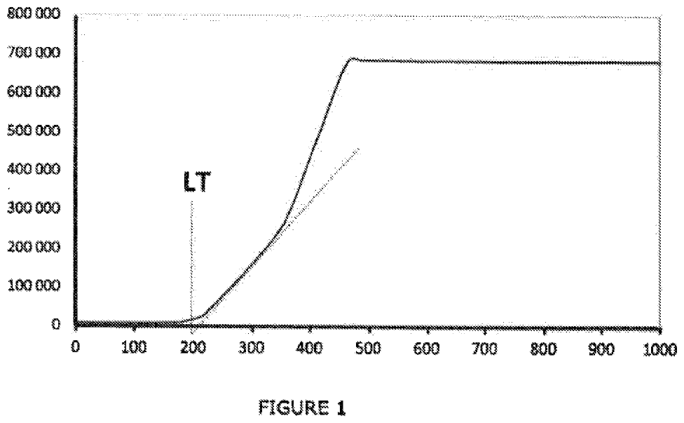

### Description of FIG. 1:

The determination of the latency time (LT) is represented graphically in this figure by monitoring the fluorescence of Thioflavin T, on a curve upon which the ordinate shows the value of the fluorescence (in a.u., arbitrary units) and the time in minutes upon the abscissa.

### EXAMPLES

Part A—Synthesis of Protected Hydrophobic Intermediates for Obtaining the Radicals [symbol] Hy

| | PROTECTED HYDROPHOBIC |
|---|---|
| No. | INTERMEDIATE COMPOUNDS |
| A1 | 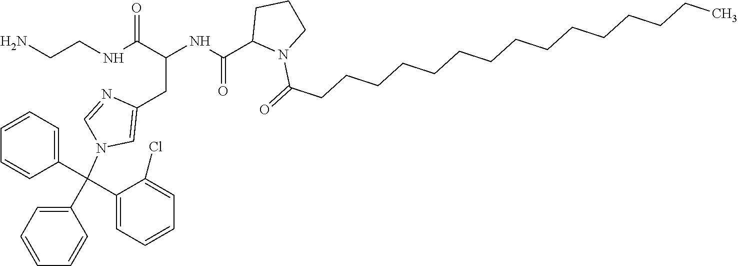 [SMILES: CCCCCCCCCCCCCCCC(=O)N1CCCC1C(=O)NC(Cc1cn(C(c2ccccc2)(c2ccccc2)c2ccccc2Cl)cn1)C(=O)NCCN] |
| A2 | 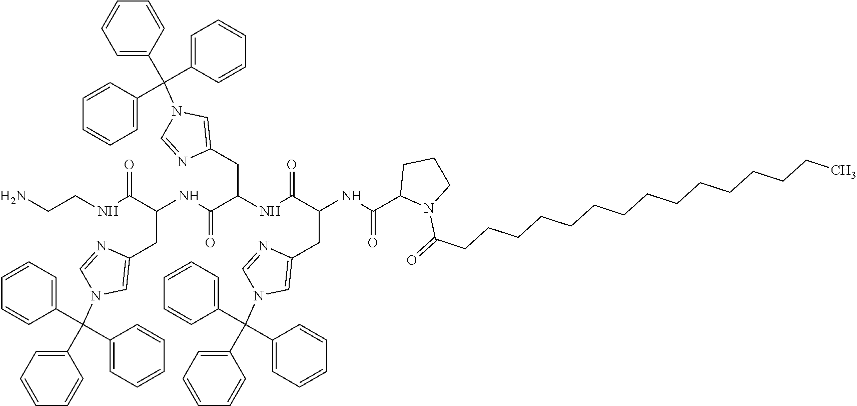 [SMILES: CCCCCCCCCCCCCCCC(=O)N1CCCC1C(=O)NC(Cc1cn(C(c2ccccc2)(c2ccccc2)c2ccccc2)cn1)C(=O)NC(Cc1cn(C(c2ccccc2)(c2ccccc2)c2ccccc2)cn1)C(=O)NC(Cc1cn(C(c2ccccc2)(c2ccccc2)c2ccccc2)cn1)C(=O)NCCN] |
| A3 | 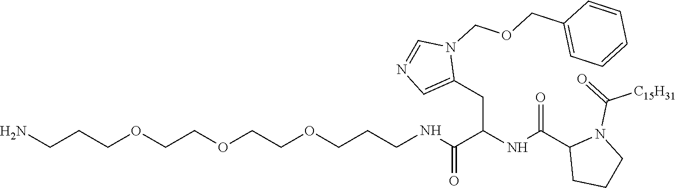 |
| A4 | 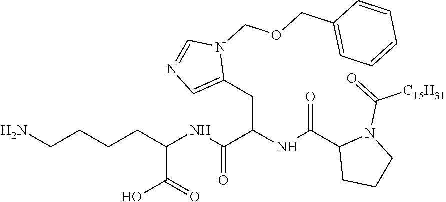 |

### Example A1: Molecule A1

### Molecule 1: Product Obtained by the Reaction Between N-Boc Ethylenediamine and Phthalic Anhydride

Phthalic anhydride (20.34 g, 137.34 mmol) is added to a solution of N-Boc ethylenediamine (BocEDA, 20.0 g, 124.83 mmol) in toluene (300 mL) at room temperature. The mixture is then heated under reflux in a Dean-Stark apparatus for 6 h. After cooling to room temperature and standing overnight, a precipitate has formed. Hexane (50 mL) is added dropwise. After 1 h, the precipitate is filtered, washed with diethyl ether (4×30 mL), then dried at 35° C. under reduced pressure. A crystalline powder is obtained from molecule 1.

Yield: 28.4 g (78%)

¹H NMR (DMSO-d6, ppm): 1.26 (9H); 3.16 (2H); 3.61 (2H); 6.54 (0.15H); 6.93 (0.85H); 7.75-7.94 (4H).

Molecule 2: Product Obtained by Reaction between Molecule 1 and Trifluoroacetic Acid.

Trifluoroacetic acid (TFA, 30.15 mL, 391.3 mmol) is added dropwise to a solution of molecule 1 (28.4 g, 97.8 mmol) in dichloromethane (DCM, 142 mL) at room temperature while maintaining the temperature of the reaction medium ≤25° C. After overnight at room temperature, hexane (142 mL) and then ethyl acetate (5 mL) is added dropwise. The precipitate is filtered, washed with diethyl ether (3×20 mL), then dried at 35° C. under reduced pressure. A solid of molecule 2 is obtained.

Yield: 18.1 g (61%)

¹H NMR (CD₃OD, ppm): 3.26 (2H); 4.00 (2H); 7.78-7.95 (4H).

### Molecule 3: Product Obtained by the Reaction Between Proline and Palmitoyl Chloride.

A solution of palmitoyl chloride (33 mL, 109.14 mmol) in methyl-THF (138 mL) is added dropwise to a solution of L-proline (25.13 g, 218.29 mmol) in a mixture of water (121.5 mL) and 10 N NaOH (27.3 mL, 272.86 mmol) at 0° C. under vigorous stirring while maintaining the temperature of the reaction medium 5° C. The reaction medium is stirred at from 4° C. to 20° C. for 1.5 h, then for 3 h at room temperature. After cooling down to 0° C., the pH is adjusted to 1.5 with concentrated hydrochloric acid (18.2 mL). The mixture is warmed to 20° C. and the phases are separated. The organic phase is washed with a 5% aqueous solution of KHSO₄ (3×100 mL), water (100 mL) and then concentrated under reduced pressure. The residue is then recrystallized from heptane (200 mL). A solid of molecule 3 is obtained.

Yield: 36.6 g (95%)

¹H NMR (CDCl_{hd 3}, ppm): 0.87 (3H); 1.15-1.41 (24H); 1.57-1.74 (2H); 1.86-2.13 (3H); 2.35 (2H); 2.41-2.53 (1H); 3.39-3.52 (1H); 3.52-3.65 (1H); 4.37-4.44 (0.05H); 4.54-4.64 (0.95H); 7.83 (1H).

Molecule 4: Product Obtained by the Reaction between Fmoc-His(ClTrt)-OH and 2-chlorotrityl chloride resin.

A solution of Fmoc-His (ClTrt)-OH (7.35 g, 11.24 mmol) in DCM (150 mL) is added to 2-Cl-trityl chloride resin (1.5 mmol/g, 15 g), which was washed beforehand with DCM (2×150 mL), then N,N-diisopropylethylamine (DIPEA, 9.8 mL, 56.19 mmol) is added. After overnight of stirring at room temperature, methanol (12 mL) is added and the medium is stirred for 15 min at room temperature. The resin is filtered, washed successively with DCM (3×150 mL), N-methyl-2-pyrrolidone (NMP, 2×150 mL), DCM (2×150 mL) and methanol (3×150 mL).

### Molecule 5: Product Obtained by a Reaction Between Molecule 4 and a 90:10 NMP/piperidine Mixture.

Molecule 4, previously washed with NMP (150 mL), is treated with a 90:10 NMP/piperidine mixture (165 mL). After 45 min of stirring at room temperature, the resin is filtered, washed successively with NMP (3×150 mL), methanol (3×150 mL) and NMP (3×150 mL).

### Molecule 6: Product Obtained by the Reaction Between Molecule 5 and Molecule 3.

1-[bis(dimethylamino) methylene]-1H-1,2,3-triazolo[4,5-b]pyridinium 3-oxide hexafluorophosphate (HATU, 12.17 g, 32.01 mmol) is added to a solution of molecule 3 (11.91 g, 33.69 mmol) in NMP (165 mL). After 30 min of stirring at room temperature, this solution is poured onto molecule 5 and DIPEA (7.8 mL, 44.92 mmol) is added. After stirring overnight at room temperature, the resin is filtered, washed successively with NMP (3×150 mL), methanol (3×150 mL) and NMP (3×150 mL).

### Molecule 7: Product Obtained by a Reaction Between Molecule 6 and a 1% TFA/DCM Mixture.

Molecule 6, washed beforehand with dichloromethane (150 mL), is treated with a 1% TFA mixture in DCM (150 mL). After 5 min of stirring at room temperature, the resin is filtered, and the solvents are evaporated under reduced pressure.

Molecule 7 is obtained in the form of a yellow oil which is used directly in the next step.

Yield: 12.1 g (reaction crude)

LC/MS (ESI+): 767.2 (calculated ([M+H]⁺): 767.4)

### Molecule 8: Product Obtained by the Reaction Between Molecule 7 and Molecule 2.

At 0° C., DIPEA (5.7 mL, 32.84 mmol), (3-dimethylaminopropyl)-N [symbol] ethylcarbodiimide hydrochloride (EDC, 2.31 g, 12.04 mmol) and N-hydroxybenzotriazole (HOBt, 1.84 g, 12.04 mmol) are successively added to a solution of molecule 7 (11.24 mmol) in DCM (84 mL). After 5 min, molecule 2 (4.0 g, 13.13 mmol) is added. Then, the reaction medium is stirred overnight at room temperature. Cold water (50 mL) is added and the phases are separated. The aqueous phase is extracted with DCM (2×50 mL). The combined organic phases are washed with a 5% aqueous solution of KHSO₄ (50 mL), a saturated aqueous solution of NaHCO₃ (50 mL) and a saturated aqueous solution of NaCl (2×50 mL). The organic phase is dried over Na₂SO₄, filtered and concentrated under reduced pressure. Molecule 8 is obtained in the form of a white solid after purification by chromatography on silica gel (eluent: DCM, methanol).

Yield: 8.5 g (80%)

¹H NMR (CDCl_{hd 3}, ppm): 0.87 (3H); 1.00-1.49 (26H); 1.84-2.23 (5H); 2.37 (1H); 2.76 (1H); 3.07 (1H); 3.24-3.90 (6H); 4.30 (1H); 4.58 (1H); 6.57 (1H); 6.83 (1H); 7.01-7.13 (4H); 7.18-7.46 (11H); 7.64 (2H); 7.81 (2H); 8.34 (1H).

LC/MS (ESI+): 939.3 (calculated ([M+H]⁺): 939.5)

### Molecule A1

A solution of molecule 8 (8.5 g, 9.05 mmol) and hydrazine monohydrate (1.32 mL, 27.14 mmol) is stirred overnight at room temperature in methyl tert-butyl ether (MTBE, 85 mL). The precipitate is filtered off and washed with MTBE (55 mL) then the filtrate is concentrated under reduced pressure. A white solid of molecule A1 is obtained after purification by chromatography on silica gel (eluent: DCM, methanol).

Yield: 5.5 g (75%)

¹H NMR (CDCl_{hd 3}, ppm): 0.88 (3H); 1.01-1.39 (28H); 1.92-2.37 (6H); 2.68-2.91 (3H); 3.01-3.27 (2H); 3.27-3.44 (1H); 3.44-3.61 (1H); 3.73-3.88 (1H); 4.40 (1H); 4.60 (1H); 6.60 (1H); 6.85 (1H); 7.02-7.21 (5H); 7.29-7.44 (10H); 8.77 (1H).

LC/MS (ESI+): 809.3 (calculated ([M+H]⁺): 809.5)

### Example A2: Molecule A2

### Molecule 9: Product Obtained by Solid Phase Peptide Synthesis.

Molecule 9 ([His(Trt)]₃ProCl6) is obtained by the conventional method of solid phase peptide synthesis on 2-chlorotrityl resin, successively using Fmoc-protected amino acids Fmoc-L-His(Trt)-OH and Fmoc-Pro-OH, then palmitic acid (5 equivalents) and diisopropylcarbodiimide (5 equivalents)/cyano (hydroxyimino) ethyl acetate (5 equivalents) as coupling agents. A 20% solution of piperidine in DMF is used for the Fmoc protecting group cleavage steps. The resin is washed with DCM, DMF and methanol after each coupling and deprotection step. Cleavage of the product of the resin is carried out using an 80:20 DCM/HFIP mixture.

### Molecule A2

By a process similar to that used for the preparation of molecule 8 and applied to molecule 9 and ethylene diamine (20 equivalents), a white solid of molecule A2 is obtained after precipitation and trituration in diethyl ether, purification by preparative HPLC (C18 column, water/acetonitrile gradient) and freeze-drying.

Yield: 0.3 g

LC/MS (ESI+): 1533.8 (calculated ([M+H]⁺): 1533.9)

### Example A3: Molecule A3

Molecule A3 is obtained by the method of solid phase peptide synthesis (SPPS) on 2-chlorotrityl resin

A solution of 4,7,10-trioxa-1,13-tridecanediamine (TOTA, 68 mL, 310 mmol) in DCM (140 mL) is poured onto 2-chlorotrityl resin (13.60 g, 1.14 mmol/g, 15.5 mmol) washed beforehand with DCM in a reactor suitable for SPPS. After 2 h stirring at room temperature, methanol (0.8 mL/g, 11 mL) is added and the medium is stirred for 15 min. The resin is filtered, washed successively with DCM, DMF, DCM, isopropanol and DCM. The protected amino acids N-Fmoc-L-Histine (3-Bom) (10.03 g, 20.2 mmol, 1.3 equivalents) and N-Fmoc-L-proline (6.80 g, 20.2 mmol, 1.3 equivalents) then palmitic acid (5.17 g, 20.2 mmol, 1.3 equivalents) are successively coupled using 1-[bis (dimethylamino)methylene]-1H-1,2,3-triazolo[4,5-b] pyridinium 3-oxide hexafluorophosphate (HATU, 1.3 equivalents) as a coupling agent in the presence of DIPEA (2.6 equivalents) in DMF. A 20% solution of piperidine in DMF is used for the Fmoc protecting group cleavage steps. The resin is washed with DCM, DMF and methanol after each coupling and deprotection step.

Cleavage of the product of the resin is carried out using a 1:1 TFA/DCM mixture. The solvents are then evaporated under reduced pressure; the residue is solubilized in DCM (500 mL) and the organic phase is washed with an aqueous solution of NaOH 1N (1×200 mL). After drying over Na₂SO₄, the organic phase is filtered, then concentrated under reduced pressure. The molecule A4 is obtained in the form of a yellow oil.

Yield: 10 g (79%)

¹H NMR (CDCl_{hd 3}, ppm): 0.88 (3H); 1.19-1.41 (24H); 1.51-2.29 (14H); 2.29 (2H); 3.06-3.18 (1H); 3.18-3.33 (3H); 3.38-3.46 (3H); 3.51-3.65 (11H); 4.43-4.54 (3H); 4.61-4.68 (1H); 5.34 (2H); 6.74-6.77 (1H); 6.86-6.95 (1H); 7.28-7.39 (6H); 7.45- 7.49 (1H).

LC/MS (ESI): 813.6; (calculated ([M+H]⁺): 813.6).

### Example A4: Molecule A4

### Molecule 10: Product Obtained by Solid Phase Peptide Synthesis.

Grafting of the first amino acid Na-Fmoc-L-Lysine(Boc) (19.26 g, 41 mmol) on the 2-chlorotrityl resin (20 g, 1.37 mmol/g, 27.4 mmol) is carried out in DCM (200 mL), in the presence of DIPEA (11.9 mL, 69 mmol). The unreacted sites are capped with methanol (0.8 mL/g, 16 mL) at the end of the reaction. The successive couplings of the amino acids N-Fmoc-L-Histine(3-Bom) (20.45 g, 41 mmol), N-Fmoc-L-proline (13.87 g, 41 mmol) and palmitic acid (10.54 g, 41 mmol), and the steps of deprotection of the Fmoc groups are carried out according to a method similar to that used for the molecule A3. Molecule 10 is obtained after cleavage of the resin with a 20% HFIP solutionin DCM, concentration under reduced pressure, elimination of the residual HFIP by co-evaporation with toluene and crystallization in acetonitrile.

Yield: 12.60 g (55%)

¹H NMR (CDCl_{hd 3}, ppm): 0.88 (3H); 1.17-2.30 (47H); 2.93-3.12 (3H); 3.12-3.25 (1H); 3.41-3.51 (1H); 3.55-3.66 (1H); 4.22-4.40 (1H); 4.40-4.51 (1H); 4.55-4.76 (3H); 4.76-5.11 (1H); 5.35-5.55 (2H); 6.56-6.81 (1H); 6.93 (1H); 7.24-7.55 (6H); 7.86-7.97 (1H); 8.91 (1H).

LC/MS (ESI): 839.5; (calculated ([M+H]⁺): 839.6).

### Molecule A4

Molecule 10 (12.60 g, 15.02 mmol) is heat-solubilized in DCM (135 mL), then a 4 M HCl solution in dioxane (19 mL, 5 equivalents) is added over 5 min at room temperature. After 2 h stirring, the reaction mixture is concentrated under reduced pressure, co-evaporated with diisopropylether (IPE) and then dissolved in water (115 mL). The pH of the solution is adjusted to 7 with a 1 M aqueous solution of NaOH (28.5 mL), then water (100 mL) is added and the product is collected by filtration through a frit, washed with water (2×50 mL) and dried under reduced pressure at 30° C. for 48 h. A white solid of the molecule A4 is obtained.

Yield: 9.81 g (88%)

¹H NMR (D₂O, ppm): 0.87 (3H); 1.09-1.55 (28H); 1.63-2.33 (10H); 3.00 (2H); 3.17- 3.65 (4H); 4.26 (1H); 4.34-4.44 (1H); 4.54-4.80 (3H); 5.65-5.94 (2H); 7.06-7.53 (6H); 8.86-9.01 (1H).

LC/MS (ESI): 739.5; (calculated ([M+H]⁺): 739.5).

Part A [symbol] —Precursors of hydrophobic compounds Hyd

| No. | PRECURSORS OF HYDROPHOBIC COMPOUNDS |
|---|---|
| Ap1 | 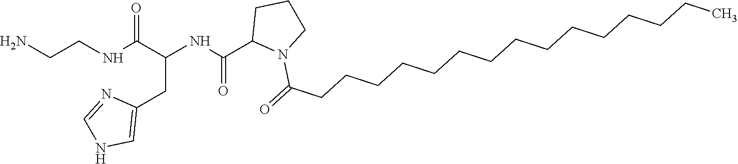 [SMILES: CCCCCCCCCCCCCCCC(=O)N1CCCC1C(=O)NC(Cc1c[nH]cn1)C(=O)NCCN] |
| Ap2 | 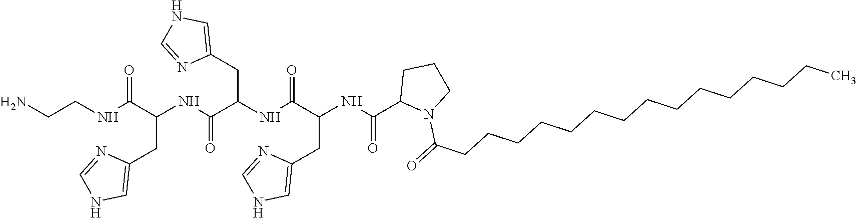 [SMILES: CCCCCCCCCCCCCCCC(=O)N1CCCC1C(=O)NC(Cc1c[nH]cn1)C(=O)NC(Cc1c[nH]cn1)C(=O)NC(Cc1c[nH]cn1)C(=O)NCCN] |
| Ap3 | 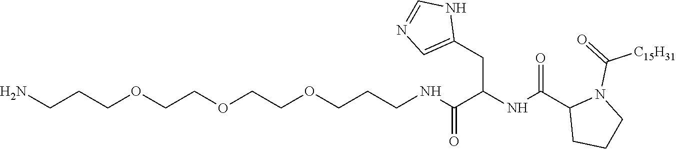 |
| Ap4 | 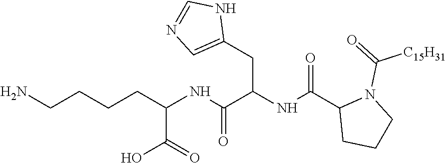 |

The cleavage of the benzyloxymethyl (BOM) and trityl (Tr) groups present on molecules A1 to A4 is carried out either by hydrogenation in the presence of Pd/Al₂O₃ or by addition of a 33% HBr solution in acetic acid. These deprotection steps are described in particular in the syntheses of the copolyamino acids B1 and B2.

### Part B—Synthesis of Hydrophobic Copolyamino Acids

| | COPOLYAMINO ACIDS BEARING CARBOXYLATE CHARGES AND |
|---|---|
| No. | HYDROPHOBIC RADICALS |
| B1 | 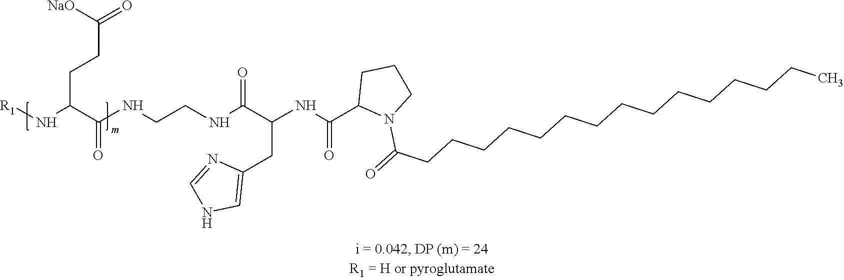 |
| B2 | 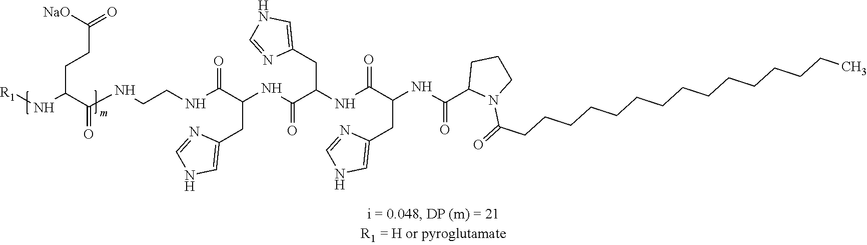 |
| B3 | 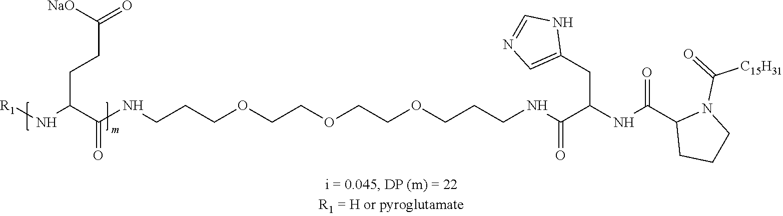 |
| B4 | 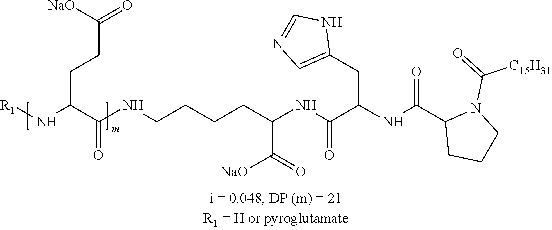 |

### Example B1: Copolyamino Acid B1—Sodium poly-L-glutamate Modified at its Extremities by Molecule A1 and having a Number-Average Molar Mass (Mn) of 3845 g/mol

Copolyamino Acid B1-1: poly-L-benzylglutamate Modified at its One of its Extremities by the Molecule A1.

In a previously oven-dried flask , y-benzyl-L-glutamate N-carboxyanhydride (39 g, 148.1 mmol) is solubilized in anhydrous DMF (80 mL). The mixture is cooled down at 4° C., and then a solution of molecule A1 (5.45 g, 6.73 mmol) in DMF (10 mL) is rapidly introduced. The mixture is stirred at from 4° C. to room temperature for 18 h, then heated to 65° C. for 2 h The reaction medium is then cooled down to room temperature and poured dropwise into diisopropyl ether (IPE, 1350 mL) under stirring. The white precipitate is recovered by filtration, washed with IPE (2×100 mL) and dried at 30° C. under reduced pressure to give a poly-L-benzylglutamate modified at both extremities by the molecule A1.

### Copolyamino Acid B1

Under an argon atmosphere, Pd/Al₂O₃ (7.2 g) is added to a solution of copolyamino acid B1-1 (36 g) in N,N-dimethylacetamide (DMAc, 360 mL). The mixture is placed under a hydrogen atmosphere (10 bar) and stirred at 60° C. for 24 h After cooling down at room temperature and filtering the catalyst through sintered glass and then through an Omnipore 0.2 μm hydrophilic PTFE membrane, a solution of water at pH 2 (2160 mL) is poured dropwise onto the DMAc solution under stirring over a period of 45 min. After 18 h under stirring, the white precipitate is recovered by filtration, washed with water (4×180 mL) and then dried under reduced pressure at 30° C. The solid (21.2 g) is suspended in TFA (130 mL) and the mixture is stirred for 24 h at room temperature and then poured dropwise onto a 1:1 (v/v) mixture of IPE/water under stirring (280 mL). After 3 h under stirring, the precipitate is recovered by filtration, washed with IPE (2×110 mL) and then dried under reduced pressure at 30° C. The solid obtained is then solubilized in water (500 mL) by adjusting the pH to 7 by adding a 1N sodium hydroxide aqueous solution. The pH is then adjusted to pH 12 and the solution is maintained under stirring for 2 h After neutralization to pH 7, the solution is filtered through a 0.2 μm filter, diluted with ethanol to obtain a solution containing ethanol at 30% mass, and then filtered through an activated carbon filter (3M R53SLP). The solution obtained is filtered through a 0.45 μm filter and purified by ultrafiltration against a 0.9% NaCl solution and then water until the conductivity of the permeate is less than 50 μS/cm. The copolyamino acid solution is then concentrated to about 30 g/L theoretical and the pH is adjusted to 7. The aqueous solution is filtered through a 0.2 μm filter and preserved at 4° C.

Dry extract: 26.0 mg/g

DP (estimated by ¹H NMR)=24 therefore i=0.042

The calculated average molar mass of copolyamino acid B1 is 4119 g/mol Aqueous HPLC-SEC (PEG Calibrator): Mn=3845 g/mol.

### Example B2: Copolyamino Acid B2-sodium poly-L-glutamate Modified at One of its Extremities by Molecule A2 and having a Number-Average Molar Mass (Mn) of 3236 g/mol

Copolyamino Acid B2-1: poly-L-benzylglutamate Modified at its one of its Extremities by the Molecule A2

By a process similar to that used for the preparation of copolyamino acid B1-1 applied to molecule A2 (0.29 g, 0.19 mmol) and to y-benzyl-L-glutamate N-carboxyanhydride (1.095 g, 4.16 mmol), the copolyamino acid B2-1 is obtained.

Copolyamino acid B2

Copolyamino acid B2-1 (1.08 g) is diluted in TFA (3.8 mL), and then the solution is cooled to 4° C. A solution of 33% HBr in acetic acid (2.7 mL, 15 mmol) is then added dropwise.

The mixture is stirred at room temperature for 3 h and then poured dropwise onto a 1:1 (v/v) mixture of IPE and water under stirring (60 mL). After 2 h of stirring, the white precipitate is recovered by filtration, washed with IPE (2×5 mL) then with water (2×5 mL). The solid obtained is then solubilized in water (20 mL) by adjusting the pH to 7 by adding 1N aqueous sodium hydroxide solution. The pH is then adjusted to pH 12 and the solution is maintained under stirring for 30 min. After neutralization to pH 7, the theoretical concentration is adjusted to 20 g/L theoretical by the addition of water (10 mL). The solution obtained is filtered through a 0.45 μm filter and purified by ultrafiltration against a 0.9% NaCl solution and then water until the conductivity of the permeate is less than 50 μS/cm. The pH is adjusted to 7. The aqueous solution is filtered through 0.2 μm and stored at 4° C.

Dry extract: 8.8 mg/g

DP (estimated by ¹H NMR)=21 therefore i=0.048

The calculated average molar mass of copolyamino acid B2 is 3940 g/mol

Aqueous HPLC-SEC (PEG Calibrator): Mn=3236 g/mol.

### Example B3: Copolyamino Acid B3—sodium poly-L-glutamate Modified at One of its Extremities by Molecule A3 and having a Number-Average Molar Mass (Mn) of 2650 g/mol

Copolyamino Acid B3-1: poly-L-benzylglutamate Modified at its One of its Extremities by the Molecule A3

By a process similar to that used for the preparation of copolyamino acid B1-1 applied to molecule A3 (5.0 g, 6.15 mmol) and to y-benzyl-L-glutamate N-carboxyanhydride (35.61 g. 135.28 mmol), the copolyamino acid B3-1 is obtained.

Copolyamino acid B3

By a process similar to that used for the preparation of copolyamino acid B2 applied to copolyamino acid B3-1, but with an additional carbon filtration step (filters R53SLP, 3M) in the presence of ethanol (30% w:w) before the ultrafiltration step, the copolyamino acid B3 is obtained.

Dry extract: 23.9 mg/g

DP (estimated by ¹H NMR)=22 therefore i=0.045

The calculated average molar mass of copolyamino acid B3 is 3977 g/mol.

Organic HPLC-SEC (PEG Calibrator): Mn =2650 g/mol.

### Example B4: Copolyamino Acid B4-sodium poly-L-glutamate Modified at One of its Extremities by Molecule A4 Whose Histidine is Deprotected and having a Mean Number Average Molecular Mass (Mn) of 1850 g/mol

Copolyamino acid B4-1: poly-L-benzylglutamate Modified at its One of its Extremities by the Molecule A4.

By a process similar to that used for the preparation of copolyamino acid B1-1 applied to molecule A4 (6.57 g, 8.63 mmol) in solution in chloroform (80 mL) and to γ-benzyl-L-glutamate N-carboxyanhydride (50 g. 190 mmol) in solution in DMF (250 mL), with a distillation step making it possible to remove the chloroform and 50% of the D1VIF before the precipitation step, the copolyamino acid B4-1 is obtained.

### Copolyamino Acid B4

By a process similar to that used for the preparation of copolyamino acid B3 applied to copolyamino acid B4-1, copolyamino acid B4 is obtained.

Dry extract: 24.5 mg/g

DP (estimated by ¹H NMR)=21 therefore i=0.048

The calculated average molar mass of copolyamino acid B4 is 3774 g/mol.

Aqueous HPLC-SEC (PEG Calibrator): Mn=1850 g/mol.

### Part C Compositions

### Example C1: Preparation of 0.6 mg/mL Pamlintide Solutions Containing m-cresol (29 mM) and Glycerin (174 mM) at pH 6.6 and pH 7.0

A 5 mg/mL concentrated pramlintide solution is prepared by dissolving pramlintide in powder form purchased from Ambiopharm. This solution is added to a concentrated solution of excipients (m-cresol, glycerin) so as to obtain the intended final composition. The final pH is adjusted to 6.6 or 7.0 ±0.1 by adding NaOH/HCl.

TABLE 1
pH and visual appearance of pramlintide 0.6 mg/mL solutions

| Solution | pH | Visual appearance of the solution |
|---|---|---|
| C1-1 | 6.6 | Clear |
| C1-2 | 7.0 | Clear |

### Example C2: Preparation of 0.6 mg/mL Pramlintide Solutions Containing 6.3 mg/mL of Copolyamino Acid B1 (1.5 mM), m-Cresol (29 mM), Glycerin (174 mM) and Various Concentrations of Zinc Chloride and Sodium Chloride at pH 7.0

A concentrated solution of copolyamino acid B1 and excipients is prepared by adding concentrated solutions of excipients (m-cresol, glycerin, NaCl, zinc chloride) to a concentrated solution of copolyamino acid B1.

A 5 mg/ml concentrated pramlintide solution is added to this concentrated solution of copolyamino acid B1 and of excipients so as to obtain the final compositions C2-1 to 2-8 (Table 2). The final pH is adjusted to 7.0±0.1 by adding NaOH/HCl.

TABLE 2
Compositions and visual appearance of pramlintide solutions at 0.6
mg/mL at pH 7.0 ± 0.1 in the presence of copolyamino acid B1 and
different concentrations of sodium chloride and zinc chloride.

| | Copolyamino acid | [NaCl] | [ZnCl₂] | Visual appearance |
|---|---|---|---|---|
| Solution | B1/Pramlintide Ratio | (mM) | (mM) | of the solution |
| C2-1 | 10 | 0 | 0 | Clear |
| C2-2 | 10 | 0 | 0.45 | Clear |
| C2-3 | 10 | 0 | 0.75 | Clear |
| C2-4 | 10 | 0 | 1.5 | Clear |
| C2-5 | 10 | 50 | 0 | Clear |
| C2-6 | 10 | 10 | 0.75 | Clear |
| C2-7 | 10 | 50 | 0.75 | Clear |
| C2-8 | 10 | 100 | 0.75 | Clear |

### Example C3: Preparation of 0.6 mg/mL Pramlintide Solutions Containing Different Concentrations of Copolyamino Acid B1, m-cresol (29 mM), Glycerin (174 mM), Sodium Chloride (100 mM), Zinc Chloride at pH 6.6 and 7.0.

By a protocol similar to that described in Example C2, solutions C3-1 to C3-4 are obtained.

TABLE 3
Compositions and visual appearance of pramlintide solutions at 0.6 mg/mL at pH 6.6 and
7.0, in the presence of variable concentrations of copolyamino acid B1, sodium chloride and zinc
chloride.

| | Concentration in | | Copolyamino | | | | Visual |
|---|---|---|---|---|---|---|---|
| | Copolyamino acid B1 | | acid/Pramlintide | [NaCl] | [ZnCl₂] | | appearance |
| Solution | mg/mL | mM | Ratio | (mM) | (mM) | pH | of the solution |
| C3-1 | 5.4 | 1.3 | 8.7 | 100 | 0.37 | 7. 0 | Clear |
| C3-2 | 8.6 | 2.1 | 14 | 100 | 0.6 | 7.0 | Clear |
| C3-3 | 5.4 | 1.3 | 8.7 | 100 | 0.37 | 6.6 | Clear |
| C3-4 | 8.6 | 2.1 | 14 | 100 | 0.6 | 6.6 | Clear |

### Example C4: Preparation of a 0.6 mg/mL Pramlintide Solution Containing 6.3 mg/mL (1.5 mM) of Copolyamino Acid B1, m-cresol (29 mM), Glycerin (174 mM), and Sodium Chloride (50 mM) and Various Divalent Cations at pH 6.6

By a protocol similar to that described in Example C2, solutions C4-1 to C4-7 are obtained.

TABLE 4
Compositions and visual appearance of pramlintide solutions at 0.6
mg/mL at pH 6.6, in the presence of variable concentrations of
copolyamino acid B1, sodium chloride and different divalent cations.

| | Copolyamino | | | | Visual |
|---|---|---|---|---|---|
| | acid B1/ | | | [Divalent | appearance |
| | Pramlintide | [NaCl] | Divalent | Ion] | of the |
| Solution | Ratio | (mM) | Ion | (mM) | solution |
| C4-1 | 10 | 50 | — | — | Clear |
| C4-2 | 10 | 50 | ZnCl₂ | 0.5 | Clear |
| C4-3 | 10 | 50 | ZnCl₂ | 1 | Clear |
| C4-4 | 10 | 50 | CaCl₂ | 0.5 | Clear |
| C4-5 | 10 | 50 | CaCl₂ | 1 | Clear |
| C4-6 | 10 | 50 | MgCl₂ | 0.5 | Clear |
| C4-7 | 10 | 50 | MgCl₂ | 1 | Clear |

### Example C4a: Preparation of 0.6 mg/mL Pramlintide Solutions and Human Insulin at 100 IU/mL Containing Different Concentrations of Copolyamino Acid B1, m-cresol (29 mM), Glycerin (174 mM), Sodium Chloride, Zinc Chloride at pH 6.6

A concentrated solution of copolyamino acid B1 and excipients is prepared by adding concentrated solutions of excipients (m-cresol, glycerin, NaCl, zinc chloride) to a concentrated solution of copolyamino acid B1.

A 5 mg/mL concentrated solution of pramlintide is added to a concentrated solution of excipients (m-cresol, glycerin, sodium chloride, zinc chloride, copolyamino acid B1). A solution of human insulin at 500 IU/mL is added to this concentrated solution of pramlintide and of excipients so as to obtain the intended final composition. The final pH is adjusted to 6.6 by adding NaOH/HCl.

TABLE 5
Compositions and visual appearance of pramlintide solutions at 0.6 mg/mL and human
insulin at 100 IU/mL at pH 6.6, in the presence of variable concentrations of copolyamino acid B1,
sodium chloride and zinc chloride.

| | Concentration in | | Copolyamino acid | | | | Visual |
|---|---|---|---|---|---|---|---|
| | Copolyamino acid B1 | | B1/Pramlintide | [NaCl] | [ZnCl₂] | | appearance |
| Solution | mg/mL | mM | Ratio | (mM) | (mM) | pH | of the solution |
| C5-1 | 15 | 3.64 | 24 | 100 | 2 | 6.6 | Clear |
| C5-2 | 20 | 4.86 | 32 | 100 | 2 | 6.6 | Clear |
| C5-3 | 20 | 4.86 | 32 | 200 | 3 | 6.6 | Clear |

### C. Physico-Chemical

Results of Visual Observations with the Mixture and Measurements of Fibrillation by ThT Principle

The formation of amyloid fibrils (defined as ordered macromolecular structures) by a peptide can lead to stability problems. These fibrils may lead to gel formation.

Thioflavin T (ThT) fluorescence monitoring is used to analyze the physical stability of solutions. Thioflavin is a small probe molecule with a characteristic fluorescence signature when bound to amyloid-like fibrils (Naiki et al. (1989) Anal. BioChem. 177, 244-249; LeVine (1999) Methods, Enzymol. 309, 274-284).

This method makes it possible to follow the formation of fibrils for low concentrations of ThT in undiluted solutions. This monitoring is carried out under conditions of accelerated stability: under stirring and at 37° C.

### Experimental Conditions

The samples were prepared just before the start of the measurement. The preparation of each composition is described in the associated example. Thioflavin T was added to the composition from a concentrated stock solution so as to induce negligible dilution of the composition. The concentration of Thioflavin T in the composition is 2 μM.

A volume of 150 μL of the composition was introduced into a well of a 96-well plate. Each composition was analyzed in three tests (triplicate) in the same plate. The plate was sealed with transparent film in order to avoid evaporation of the composition.

This plate was then placed in the enclosure of a plate reader (EnVision 2104 Multilabel, Perkin Elmer). The temperature is set at 37° C., and lateral stirring of 960 rpm with 1 mm of amplitude is imposed.

A reading over time of the fluorescence intensity in each well is taken with an excitation wavelength of 442 nm, and an emission wavelength of 482 nm.

The process of fibrillation manifests itself as a sharp increase in fluorescence after a period called lag time.

For each well, this delay was determined graphically as the intersection between the baseline of the fluorescence signal and the slope of the fluorescence curve as a function of the determined time during the initial sharp increase in fluorescence. The reported latency value corresponds to the average of the lag time measurements made on three wells.

An example of a graphical determination is shown in FIG. 1.

The determination of the lag time (LT) is represented graphically in this figure by monitoring the fluorescence of Thioflavin T, on a curve having on the y-axis the fluorescence value (in a.u arbitrary units) and on the x-axis the time in minutes.

### Example C5: Stability of 0.6 mg/mL Solutions of Pramlintide at pH 7.0 in the Presence of Copolyamino Acid B1 at 6.3 mg/mL, m-cresol (29 mM), Glycerin (174 mM), Zinc Chloride and Sodium Chloride

TABLE 6
Measurement of the lag time by ThT of solutions C1-2 and
C2-1, C2-3, C2-5, C2-7 and C2-8.

| | [NaCl] | [ZnCl₂] | Lag |
|---|---|---|---|
| Solution | (mM) | (mM) | Time (h) |
| C1-2 | — | — | <1 |
| C2-1 | 0 | 0 | <1 |
| C2-3 | 0 | 0.75 | >1 |
| C2-5 | 50 | 0 | >2 |
| C2-7 | 50 | 0.75 | >15 |
| C2-8 | 100 | 0.75 | >15 |

The pramlintide solution at pH 7.0 (C1-2) without copolyamino acid has a short lag time. Likewise, in the presence of copolyamino acid B1 in the absence of salt, solution C2-1 has a short lag time. The combination of zinc and NaCl leads to a significant increase in lag times.

### Example C6: Stability of 0.6 mg/mL Solutions of Pramlintide at pH 6.6 and 7.0 in the Presence of Different Concentrations of Copolyamino Acid B1, m-cresol (29 mM), Glycerin (174 mM), Zinc Chloride and Sodium Chloride (100 mM)

TABLE 7
Measurement of the lag time by ThT of solutions C1-1 and C1-2 and C5-1 to C5-4.

| | | Concentration in | | Copolyamino | | | | |
|---|---|---|---|---|---|---|---|---|
| | | Copolyamino | | acid/ | | | | |
| | Copolyamino | acid | | Pramlintide | [NaCl] | [ZnCl₂] | | Lag Time |
| Solution | acid | mg/mL | mM | Ratio | (mM) | (mM) | pH | (h) |
| C1-1 | — | — | — | — | — | — | 6.6 | <1 |
| C1-2 | — | — | — | — | — | — | 7.0 | <1 |
| C5-1 | B1 | 5.4 | 1.3 | 8.7 | 100 | 0.37 | 7.0 | >40 |
| C5-2 | B1 | 8.6 | 2.1 | 14 | 100 | 0.6 | 7.0 | >60 |
| C5-3 | B1 | 5.4 | 1.3 | 8.7 | 100 | 0.37 | 6.6 | >40 |
| C5-4 | B1 | 8.6 | 2.1 | 14 | 100 | 0.6 | 6.6 | >60 |

The pramlintide solutions at pH 6.6 and 7.0 (C1-1 and C1-2) without copolyamino acid have a very short lag time; the lag times of the solutions adjusted to pH 6.6 and 7.0 containing the copolyamino acid B1 and NaCl in combination with ZnCl₂ are greater. Moreover, increasing the zinc concentration makes improvement of the lag time of the compositions possible.

### Example C7: Stability of 0.6 mg/mL Solutions of Pramlintide at pH 6.6 in the Presence of Copolyamino Acid B1 at 6.3 mg/mL, m-cresol (29 mM), Glycerin (174 mM), Zinc and Sodium Chloride (50 mM) and Different Divalent Cations

TABLE 8
Measurement of the lag time by ThT of solutions C6-1 to C6-3,
C6-5 and C6-7.

| | [NaCl] | Nature of the | [Divalent Ion] | Lag |
|---|---|---|---|---|
| Solution | (mM) | Divalent Cation | (mM) | Time (h) |
| C6-1 | 50 | — | — | 5.5 |
| C6-2 | 50 | ZnCl₂ | 0.5 | 18.5 |
| C6-3 | 50 | ZnCl₂ | 1 | 34.2 |
| C6-5 | 50 | CaCl₂ | 1 | 11.3 |
| C6-7 | 50 | MgCl₂ | 1 | 10.2 |

The lag times of the compositions containing divalent cations are better than that of the composition without divalent cation (C6-1). The lag times of compositions containing zinc ions are greater compared to compositions containing calcium or magnesium ions.

### D. Study of the Stability of the Compositions According to the Invention Visual Inspection Procedure:

3 mL vials or cartridges filled with 1 mL of formulation are visually inspected for the appearance of visible particles or turbidity. This inspection is performed according to the recommendations of the European Pharmacopoeia (EP 2.9.20): The vials are subjected to illumination of at least 2000 Lux and are observed in front of a white background and a black background. The number of weeks or months of stability corresponds to the time after which the solutions contain visible particles or are turbid.

These results are in agreement with the US pharmacopoeia (U.S. Pat. No. <790>).

### Example D1: Physical Stability at 30° C. and 37° C. in Cartridges of Solutions of Pramlintide at 0.6 mg/ml and Human Insulin at 100 IU/ml Containing Different Concentrations of Copolyamino Acid B1, m-cresol (29 mM) Glycerin (174 mM), Sodium Chloride, Zinc Chloride at pH 6.6

Solutions C5-1, C5-2 and C5-3 are filtered (0.22 μm). 1 mL of solution is introduced into a 3 mL glass cartridge using an auto-injector pen. The cartridges are placed in a static oven at 30° C. or 37° C. The cartridges are observed weekly.

TABLE 9
Results of the physical stabilities at 30° C. and 37° C. in a cartridge
of the compositions of pramlinitide at 0.6 mg/mL and human insulin
at IU/mL 100 in the presence of copolyamino acid B1.

| | | | | | Physical | Physical |
|---|---|---|---|---|---|---|
| | Concentration | | | | stabilities | stabilities |
| | in | | | | at 30° C. | at 37° C. |
| | Copolyamino | | | | in | in |
| | acid B1 | [NaCl] | [ZnCl₂] | | cartridges | cartridges |
| Solution | mg/mL | (mM) | (mM) | pH | (weeks) | (weeks) |
| C5-1 | 15 | 100 | 2 | 6.6 | >9 | >9 |
| C5-2 | 20 | 100 | 2 | 6.6 | >9 | >9 |
| C5-3 | 20 | 200 | 3 | 6.6 | >9 | >9 |

In the absence of copolyamino acid B1, the solution comprising pramlintide and insulin is turbid at pH 6.6. Solutions of pramlintide at 0.6 mg/ml and human insulin at 100 IU/ml at pH 6.6 in the presence of copolyamino acid B1, ZnCl2 and NaCl in cartridges exhibit physical stability of at least 9 weeks at 30° C. as well as 37° C.

1. Composition, in the form of an injectable solution, comprising:
amylin, an amylin receptor agonist or an amylin analogue,
at least one ionic species, and
an amphiphilic compound comprising a hydrophilic backbone HB, substituted by at least one hydrophobic radical -Hy according to the following formula I:
*-(GpR)ᵣ-(GpI)ᵢ-[(GpR)_{r′}-(GpI)_{i′}]ₜ-GpC Formula I
wherein,
GpI is a divalent radical, said radical comprising at least one imidazole Im unit according to formula III: 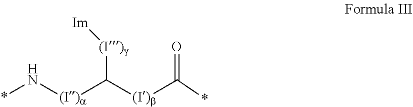
GpR is a radical according to formulas II, II′ or II″: 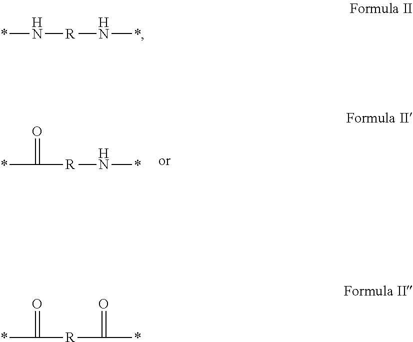
GpC is a radical according to Formula IV: 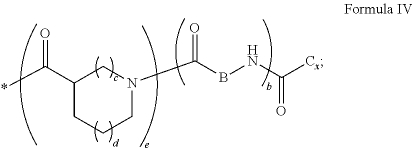
the * indicate the attachment sites of the hydrophobic radical -Hy to the hydrophilic backbone HB or the above radicals (I, II, II′, II″, III and IV) with each other via amide functions;
α, β and γ are identical or different integers equal to 0 or 1;
b is an integer equal to 0 or to 1;
c is an integer equal to 0 or 1;
d is an integer equal to 0, 1 or 2; and if c is equal to 0 then d is equal to 1 or 2;
e is an integer equal to 0 or to 1;
i and i′, whether they are identical or different, are integers less than or equal to 6 and i+i′ is greater than or equal to 1 and less than or equal to 6, 1≤i+i′≤6,
r and r′ are integers equal to 0, 1, 2 or 3;
if r is equal to 0 then the hydrophobic radical according to formula I is bound to the hydrophilic backbone HB via a covalent bond between a carbonyl of the hydrophobic radical and a nitrogen atom of the hydrophilic backbone HB, thus forming an amide function resulting from the reaction of an amine function of the precursor of the hydrophilic backbone HB and an acid function borne by the precursor of the hydrophobic radical, and
if r is equal to 1, 2 or 3 then the hydrophobic radical -Hy according to formula I is bound to the hydrophilic backbone HB:
via a covalent bond between a nitrogen atom of the hydrophobic radical and a carbonyl of the hydrophilic backbone HB, thus forming an amide function resulting from the reaction of an amine function of the precursor of the hydrophobic radical and an acid function borne by the precursor of the hydrophilic backbone HB or
via a covalent bond between a carbonyl of the hydrophobic radical and a nitrogen atom of the hydrophilic backbone HB, thus forming an amide function resulting from the reaction of an acid function of the precursor of the hydrophobic radical and an amine function of the precursor of the hydrophilic backbone HB;
t is an integer equal to 0 or to 1;
B is a linear or branched alkyl radical, optionally comprising an aromatic nucleus, comprising from 1 to 9 carbon atoms, or an unsubstituted ether or polyether radical comprising from 4 to 14 carbon atoms and from 1 to 5 oxygen atoms;
Cₓ is a linear or branched monovalent alkyl radical, optionally comprising a cyclic part, wherein x indicates the number of carbon atoms and 11≤x≤25;
I′, I″ and I′″, whether they are identical or different, are divalent radicals, chosen in the group consisting of a linear or branched alkyl radical, comprising from 1 to 12 carbon atoms,
I is a trivalent radical chosen in the group consisting of a linear or branched alkyl radical, comprising from 1 to 12 carbon atoms,
Im is an imidazolyl radical,
R is a radical chosen in the group consisting of a linear or branched divalent alkyl radical, comprising from 1 to 12 carbon atoms, a branched alkyl radical of 1 to 8 carbon atoms, said alkyl radical bearing one or more free carboxylic acid function(s), a divalent, linear or branched alkyl radical comprising from 1 to 12 carbon atoms bearing one or more functions —CONH₂ or an unsubstituted ether or polyether radical comprising from 4 to 14 carbon atoms and from 1 to 5 oxygen atoms, said free carboxylic acid functions being in the form of alkaline cation salts chosen in the group consisting of Na⁺ and K⁺, and
when several hydrophobic radicals are borne by a hydrophilic HB backbone, then they are identical or different.

2. Composition according to claim 1, wherein the hydrophobic radical -Hy is chosen among according to formula I wherein radical according to formula III is chosen among radicals according to formula IIIa:

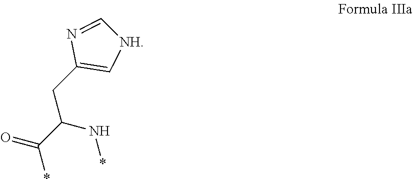 [SMILES: *NC(Cc1c[nH]cn1)C(*)=O]

3. Composition according to claim 1, wherein said at least one ionic species is chosen among cations that are at least divalent, anions, cations or zwitterions and mixtures thereof.

4. Composition according to claim 1, wherein said at least one ionic species is chosen among cations that are at least divalent.

5. Composition according to claim 1, wherein said at least one ionic species is chosen among anions, cations or zwitterions that are different from the at least divalent cations.

6. Composition according to claim 1, wherein the pH is comprised from 6.0 to 8.0.

7. Composition according to claim 1, wherein it further comprises prandial insulin.

8. Composition according to claim 1, wherein it also comprises GLP-1, analogues of GLP-1, GLP-1 receptor agonists, commonly called GLP-1 RA.

9. Composition according to claim 1, wherein the hydrophilic backbone HB is a copolyamino acid PLG bearing hydrophobic radicals, said hydrophilic backbone is chosen among the copolyamino acids according to the following formula XXX: 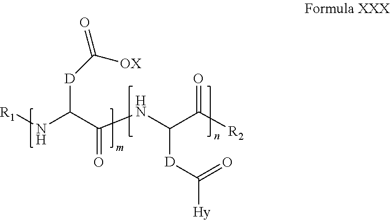
wherein,
D is, independently, either a —CH₂— group (aspartic unit) or a —CH₂—CH₂— group (glutamic unit),
R₁ is a hydrophobic radical chosen among the hydrophobic -Hy radicals, or a radical, chosen in the group consisting of an H, a linear acyl group in C₂ to C₁₀, a branched acyl group in C₃ to C₁₀, a benzyl, a terminal “amino acid” unit and a pyroglutamate,
R₂ is either a hydrophobic radical chosen among the hydrophobic radicals -Hy, or a radical chosen in the group consisting of an —OH, an amine group, a terminal “amino acid” unit and a pyroglutamate,
said copolyamino acid comprises at least one hydrophobic radical -Hy as defined above,
X represents a cationic entity chosen among the group comprising alkaline cations;
if n=0 then m≥1
if m=0 then n≥1
n+m represents the degree of polymerization DP of the copolyamino acid, that is to say, the average number of monomeric units per copolyamino acid chain and 5≤n+m≤250 and
the ratio M between the number of hydrophobic radicals and the number of glutamic or aspartic units bein comprised from 0≤M≤0.5

10. Composition according to claim 1, wherein the copolyamino acid bearing hydrophobic radicals is chosen among the copolyamino acids according to formula XXX wherein n=0 according to the following formula XXXe: 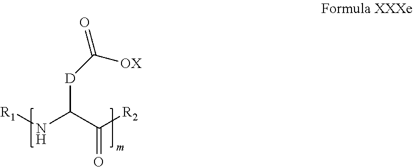
wherein
D is, independently, either a —CH₂— group (aspartic unit) or a —CH₂—CH₂— group (glutamic unit),
R₁ is a hydrophobic radical chosen among the hydrophobic radicals Hy, or a radical, chosen among the group constituted of a linear acyl group in C₂ to C₁₀, a branched acyl group in C₃ to C₁₀, a benzyl, a terminal “amino acid” unit and a proglutamate,
R₂ is a hydrophobic radical chosen among the hydrophobic radicals -Hy, or a radical chosen in the group consisting of an —OH, an amine group, a terminal “amino acid” unit and a pyroglutamate,
m represents the degree of polymerization DP of the copolyamino acid,
said copolyamino acid comprises at least one the hydrophobic radicals -Hy,
X represents a cationic entity chosen among the group comprising alkaline cations
and at least R₁ or R₂ is a hydrophobic radical Hy.

11. Composition according to claim 1, wherein the copolyamino acid bearing hydrophobic radicals is chosen among the copolyamino acids according to formula XXX wherein m=0 according to the following formula XXXf: 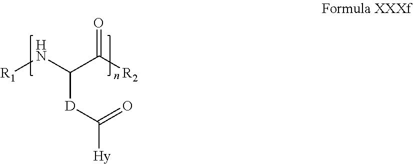
wherein
D is, independently, either a —CH₂— group (aspartic unit) or a —CH₂—CH₂— group (glutamic unit),
R₁ is a hydrophobic radical chosen among the hydrophobic radicals -Hy, or a radical, chosen among the group consisting by a linear acyl group in C₂ to C₁₀, a branched acyl group in C₃ to C₁₀, a benzyl, a terminal “amino acid” unit and a pyroglutamate,
R₂ is either a hydrophobic radical chosen among the hydrophobic radicals -Hy, or a radical chosen in the group consisting of an —OH, an amine group, a terminal “amino acid” unit and a pyroglutamate,
said copolyamino acid comprises at least one of the hydrophobic radicals -Hy,
X represents a cationic entity chosen among the group comprising alkaline cations,
n represents the degree of polymerization DP of the copolyamino acid.

12. Composition according to claim 1, wherein the hydrophilic backbone HB is a polylysine bearing hydrophobic radicals and said hydrophilic backbone is chosen among the polylysines according to the following formula XXXX: 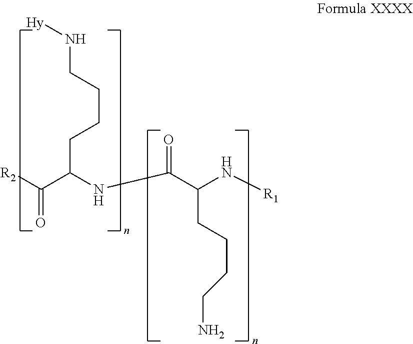
wherein,
R₁ is a hydrophobic radical chosen among the hydrophobic radicals -Hy, or a radical, chosen in the group consisting of an —H or a terminal “amino acid” unit,
R₂ is either a hydrophobic radical chosen among the hydrophobic radicals -Hy, or a radical chosen in the group consisting of an —OH, an amine group or a terminal “amino acid” unit,
said polylysine comprises at least one of the hydrophobic radicals -Hy,
if n=0 then m≥1
if m=0 then n≥1
n+m represents the degree of polymerization DP of the polylysine, that is to say, the average number of monomeric units per copolyamino acid chain and 5≤n+m≤250 and
the ratio M between the number of hydrophobic radicals and the number of repetition units being comprised from 0<M<0.5.

13. Composition according to claim 1, wherein the copolyamino acid bearing at least one hydrophobic radical -Hy is chosen among the copolyamino acids according to the following formula XXXa′: 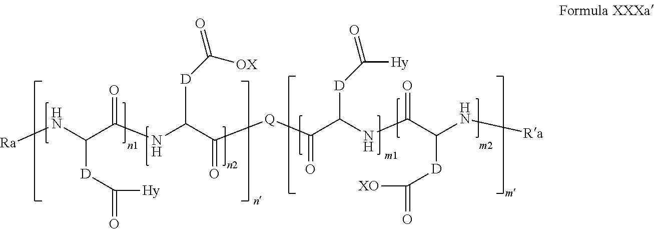
wherein:
D is, independently, either a —CH₂— group (aspartic unit) or a —CH₂—CH₂— group (glutamic unit),
Ra and R′a, whether they are identical or different, are either a hydrophobic radical -Hy, or a radical chosen among the group constituted of an H, a linear acyl group in C₂ to C₁₀, a branched acyl group in C₃ to C₁₀, a benzyl, a terminal “amino acid” unit and a pyroglutamate,
Hy is as previously define
Q being a spacer binding at least two chains of linear or branched glutamic or aspartic PLG units according to formula Q[-*]k at least divalent consisting of an alkyl chain comprising one or more heteroatoms chosen in the group consisting of nitrogen and oxygen atoms and/or bearing one or more heteroatoms consisting of nitrogen and oxygen atoms and/or radicals bearing one or more heteroatoms consisting of nitrogen atoms and oxygen and/or carboxyl functions and optionally bearing at least one hydrophobic radical -Hy.
X represents a cationic entity chosen in the group comprising alkaline cations,
n₁+m₁ represents the number of glutamic units or aspartic units of the copolyamino acid chains bearing an -Hy radical,
n₂+m₂ represents the number of glutamic units or aspartic units of the copolyamino acid chains not bearing an -Hy radical,
n₁+n₂=n′ and m₁+m₂=m′
n′+m′ represents the degree of polymerization DP of the copolyamino acid, that is to say, the average number of monomeric units per copolyamino acid chain and 5≤n′+m′≤250.

14. Composition according to claim 1, wherein the copolyamino acid bearing at least one hydrophobic radical -Hy is chosen among the following copolyamino acids according to formula XXXb′: 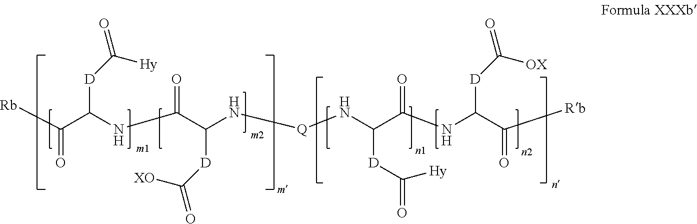
wherein:
D is independently, either a —CH₂— group (aspartic unit) or a —CH₂—CH₂— group (dutamic unit), and X represents a cationic entity chosen in the group comprising alkaline cations,
Q being a spacer binding at least two chains of linear or branched glutamic or aspartic units according to formula Q[-*]k at least divalent consisting of an alkyl chain comprising one or heteroatoms chosen in the group consisting of nitrogen and oxygen atoms and/or bearing one or more heteroatoms consisting of nitrogen and oxygen atoms and/or radicals bearing one or more heteroatoms consisting of nitrogen atoms and oxygen and/or carboxyl functions and optionally bearing at least one h drophobic radical -Hy, and Hy is as previously defined,
Rb and R′b, whether they are identical or different, are either a hydrophobic radical -Hy, or a radical chosen in the group consisting of —OH, an amine group, a terminal “amino acid” unit and a pyroglutamate,
at least one of Rb and R′b is a hydrophobic radical -Hy,
n₁+m₁ represents the number of glutamic units or aspartic units of the copolyamino acid chains bearing an -Hy radical,
n₂+m₂ represents the number of glutamic units or aspartic units of the copolyamino acid chains not bearing an -Hy radical,
n₁+n₂=n′ and m1+m2=m′
n′+m′ represents the degree of polymerization DP of the copolyamino acid, that is to say, the average number of monomeric units per copolyamino acid chain and 5≤n′+m′≤250.

15. Composition according to claim 1, wherein the hydrophilic backbone HB is a polyalkylene glycol bearing hydrophobic radicals and said hydrophilic backbone is chosen among among the polyalkylene glycols according to the following formula XXXXXa 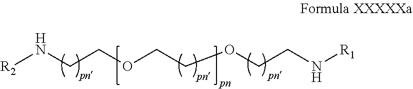
wherein,
R₁ is a hydrophobic radical chosen among the hydrophobic radicals Hy, or a radical, chosen in the group consisting of an —H or —OH,
R₂ is either a hydrophobic radical chosen among the hydrophobic radicals -Hy, or a radical chosen in the group consisting of an —OH or —H,
and at least one among R₁ or R₂ is a hydrophobic radical -Hy,
pn′ is an integer from 1 to 5, 1≤pn′≤5
pn represents the degree of polymerization DP of the polyalkylene glycol, that is to say, the average number of monomer units per polyalkylene glycol chain and 5≤n+m≤250.

16. Amphiphilic compounds comprising a hydrophilic backbone HB, substituted by at least one hydrophobic radical -Hy according to the following formula I:
*-(GpR)ᵣ-(GpI)ᵢ-[(GpR)_{r′}-(GpI)_{i′}]ₜ-GpC Formula I
wherein,
GpI is a divalent radical, said radical comprising at least one imidazole Im unit according to formula III: 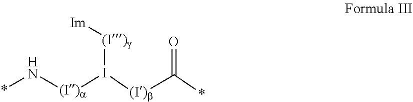
GpR is a radical according to formulas II, II′ or II″: 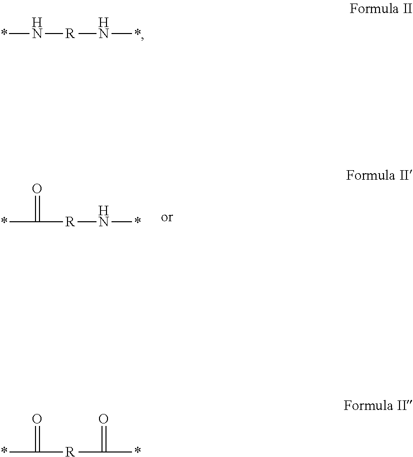
GpC is a radical according to Formula IV: 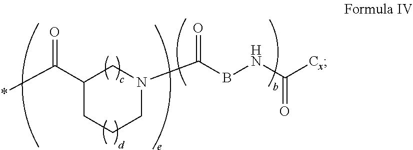
the * indicate the attachment sites of the hydrophobic radical -Hy to the hydrophilic backbone HB or the above radicals (I, II, II′, II″, III and IV) with each other via amide functions;
α, β and γ are identical or different integers equal to 0 or 1;
b is an integer equal to 0 or to 1;
c is an integer equal to 0 or 1;
d is an integer equal to 0, 1 or 2; and if c is equal to 0 then d is equal to 1 or 2;
e is an integer equal to 0 or to 1;
i and i′, whether they are identical or different, are integers less than or equal to 6 and i+i′ is greater than or equal to 1 and less than or equal to 6, 1 i+i′≤6,
r and r′ are integers equal to 0, 1, 2 or 3;
if r is equal to 0 then the hydrophobic radical according to formula I is bound to the hydrophilic backbone HB via a covalent bond between a carbonyl of the hydrophobic radical and a nitrogen atom of the hydrophilic backbone HB, thus forming an amide function resulting from the reaction of an amine function of the precursor of the hydrophilic backbone HB and an acid function borne by the precursor of the hydrophobic radical, and
if r is equal to 1, 2 or 3 then the hydrophobic radical -Hy according to formula I is bound to the hydrophilic backbone HB:
via a covalent bond between a nitrogen atom from the hydrophobic radical and a carbonyl of the hydrophilic backbone HB, thus forming an amide function resulting from the reaction of an amine function of the precursor of the hydrophobic radical and an acid function borne by the hydrophilic backbone HB or
via a covalent bond between a carbonyl of the hydrophobic radical and a nitrogen atom of the hydrophilic backbone HB, thus forming an amide function resulting from the reaction of an acid function of the precursor of the hydrophobic radical and an amine function of the precursor of the hydrophilic backbone HB;
t is an integer equal to 0 or to 1;
B is a linear or branched alkyl radical, optionally comprising an aromatic ring comprising from 1 to 9 carbon atoms or an unsubstituted ether or polyether radical comprising from 4 to 14 carbon atoms and from 1 to 5 oxygen atoms;
Cₓ is a linear or branched monovalent alkyl radical, optionally comprising a ring portion, wherein x indicates the number of carbon atoms and 11≤x≤25:
I′, I″ and I′″, whether they are identical or different, are divalent radicals, chosen in the group consisting of a linear or branched alkyl radical, comprising from 1 to 12 carbon atoms,
I is a trivalent radical, chosen in the group consisting of a linear or branched alkyl radical, comprising from 1 to 12 carbon atoms,
Im is an imidazolyl radical,
R is a radical chosen in the group consisting of a linear or branched divalent alkyl radical, comprising from 1 to 12 carbon atoms, a branched alkyl radical of 1 to 8 carbon atoms, said alkyl radical bearing one or more free carboxylic acid function(s), a divalent linear or branched alkyl radical comprising from 1 to 12 carbon atoms bearing one or more functions —CONH₂ or an unsubstituted ether or polyether radical comprising from 4 to 14 carbon atoms and from 1 to 5 oxygen atoms, said free carboxylic acid functions being in the form of alkaline cation salts chosen in the group consisting of Na⁺ and K⁺, and
when several hydrophobic radicals are borne by a hydrophilic HB backbone, then they are identical or different.

17. Precursor Hy′ of the hydrophobic radical -Hy according to formula I′ as defined below:
H-(GpR)ᵣ-(GpI)ᵢ-[(GpR)_{r′}-(GpI)_{i′}]ₜ-GpC Formula I′
wherein,
GpI is a divalent radical, said radical comprising at least one imidazole Im unit according to formula III: 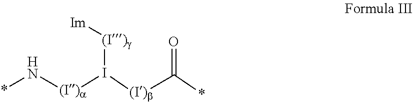
GpR is a radical according to formulas II, II′ or II″: 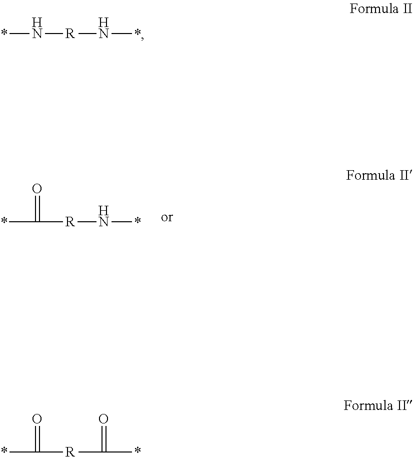
GpC is a radical according to Formula IV: 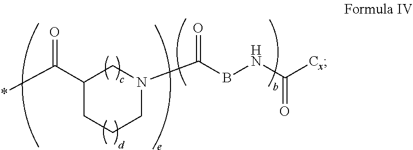
the * indicate the attachment sites of the hydrophobic radical -Hy to the hydrophilic backbone HB or the above radicals (I, II, II′, II″, III and IV) with each other via amide functions;
α, β and γ are identical or different integers equal to 0 or 1;
b is an integer equal to 0 or to 1;
c is an integer equal to 0 or 1;
d is an integer equal to 0, 1 or 2; and if c is equal to 0 then d is equal to 1 or 2;
e is an integer equal to 0 or to 1;
i and i′, whether they are identical or different, are integers less than or equal to 6 and i+i′ is greater than or equal to 1 and less than or equal to 6, 1≤i+i′≤6,
r and r′ are integers equal to 0, 1, 2 or 3;
if r is equal to 0, then the hydrophobic radical according to formula I is bound to the hydrophilic backbone HB via a covalent bond between a carbonyl of the hydrophobic radical and a nitrogen atom of the hydrophilic backbone HB, thus forming an amide function resulting from the reaction of an amine function of the precursor of the hydrophilic backbone HB and an acid function borne by the precursor of the hydrophobic radical, and
if r is equal to 1, 2 or 3 then the hydrophobic radical -Hy according to formula I is bound to the hydrophilic backbone HB:
via a covalent bond between a nitrogen atom of the hydrophobic radical and a carbonyl of the hydrophilic backbone HB, thus forming an amide function resulting from the reaction of an amine function of the precursor of the hydrophobic radical and
an acid function borne by the precursor of the hydrophilic backbone HB or
via a covalent bond between a carbonyl of the hydrophobic radical and a nitrogen atom of the hydrophilic backbone HB, thus forming an amide function resulting from the reaction of an acid function of the precursor of the hydrophobic radical and an amine function of the precursor of the hydrophilic backbone HB;
t is an integer equal to 0 or to 1;
B is a linear or branched alkyl radical, optionally comprising an aromatic ring comprising from 1 to 9 carbon atoms or an unsubstituted ether or polyether radical comprising from 4 to 14 carbon atoms and from 1 to 5 oxygen atoms;
Cₓ is a linear or branched monovalent alkyl radical, optionally comprising a ring portion, wherein x indicates the number of carbon atoms and 11≤x≤25:
I′, I″ and I′″, whether they are identical or different, are divalent radicals, chosen in the group consisting of a linear or branched alkyl radical, comprising from 1 to 12 carbon atoms,
I is a trivalent radical, chosen in the group consisting of a linear or branched alkyl radical, comprising from 1 to 12 carbon atoms,
Im is an imidazolyl radical,
R is a radical chosen in the group consisting of a linear or branched divalent alkyl radical, comprising from 1 to 12 carbon atoms, a branched alkyl radical of 1 to 8 carbon atoms, said alkyl radical bearing one or more free carboxylic acid function(s), a divalent linear or branched alkyl radical comprising from 1 to 12 carbon atoms bearing one or more functions —CONH₂ or an unsubstituted ether or polyether radical comprising from 4 to 14 carbon atoms and from 1 to 5 oxygen atoms; said free carboxylic acid functions being in the form of alkaline cation salts chosen in the group consisting of Na⁺ and K⁺, and
when several hydrophobic radicals are borne by a hydrophilic HB backbone, then they are identical or different.

18. (canceled)
